(19) United States
(12) Patent Application Publication
(10) Pub. No.: US 2004/0221332 A1
(43) Pub. Date: Nov. 4, 2004

(54) Plant growth regulating genes, proteins and uses thereof

(76) Inventors: Rene Lorbiecke, Quickborn (DE); Vladimir Mironov, Sint-Amandsberg (BE); Valerie Frankard, Brussel (BE); Willy Dillen, Sint-Amandsberg (BE); Pierre LeJeune, Dolembreux (BE); Willem Broekaert, Dilbeek (BE); Margret Sauter, Hamburg (DE)

Correspondence Address: NIXON & VANDERHYE, PC, 1100 N GLEBE ROAD, 8TH FLOOR, ARLINGTON, VA, 22201-4714, US

(21) Appl. No.: 10/474,691

(22) PCT Filed: Apr. 11, 2002
(86) PCT No.: PCT/EP02/04035

Related U.S. Application Data
(60) Provisional application No. 60/283,313, filed on Apr. 12, 2001.

(51) Int. Cl. A01H 1/00; C12N 15/82; C07H 21/04

(52) U.S. Cl. 800/279; 530/370; 536/23.6

(57) ABSTRACT

Provided are methods for controlling or altering the growth characteristics of a plant or of an organ or tissue or cell of this plant. This method comprises introduction and/or expression of one or more Growth Regulating Proteins functional in a plant or parts thereof and/or one or more DNA sequences encoding such proteins. Another method comprises contacting a plant or plant tissue with the purified Growth Regulated Protein or the active product derived from it. Provided are also DNA sequences encoding plant Growth Regulating Protein(s) as well as methods for obtaining the same. Furthermore, vectors comprising said DNA sequences are described, wherein the DNA sequences are operatively linked to regulatory elements allowing expression in prokaryotic and/or eukaryotic host cells. In addition, proteins encoded by said DNA sequences, antibodies to said proteins and methods for their production are provided. Furthermore, regulatory sequences, which naturally regulate the expression of the above-described DNA sequences, are described. Methods for the identification of compounds being capable of interacting with said Growth Regulating Proteins are described as well. Furthermore, transgenic plant cells, plant tissues and plants containing the above-described DNA sequences and vectors are described, as well as the use of the aforementioned DNA sequences, vectors, proteins, antibodies, regulatory sequences and/or compounds identified by the method of the invention in plant cell and tissue culture, plant breeding and/or agriculture.

### FIELD OF THE INVENTION

The present invention relates to methods and compositions for regulating the growth characteristics of a plant, including a cell, tissue or organ of the plant, using isolated nucleic acid sequences encoding growth regulating proteins (GREPs) and the corresponding GREPs.

### BACKGROUND OF THE INVENTION

Rice (Oryza sativa) can be cultivated in different ecosystems depending on the water supply. Deepwater rice is semi-aquatic and distinguishes itself from most other cultivated varieties in its ability to survive flooding for extended periods of time. The so-called floating rice types can exhibit extreme elongation and, when partially submerged, can grow at rates up to 25 cm/day, reaching a length of up to 7 m in water depths of 4 m (Kende et al., 1998). When deepwater rice plants are flooded, growth of the youngest internode accelerates to keep the uppermost leaves above the rising water level. At the same time, pre-existent adventitious root primordia that are located around the nodes emerge through the nodal meristem and develop further to provide nutrients to the newly developing aerial parts of the plant. Because of its unique biological properties, deepwater rice is particularly well suited for studying basic aspects of plant growth at the cellular, physiological, and biochemical level. Deepwater rice thus provides a model system for the identification of genes involved in growth-related processes. In general, internodal growth of deepwater rice has been studied in detail in the past (Kende et al., 1998; Lorbiecke & Sauter, 1998). These studies showed that the plant hormones gibberellic acid (GA), ethylene and abscisic acid (ABA) play an important role in triggering accelerated growth of internodes and adventitious roots upon flooding. In the internode, GA is the immediate growth-promoting hormone. Ethylene is only an intermediate player in the signal transduction pathway that leads to internodal growth; ethylene leads to an increased concentration of GA in the tissue and also to an increased responsiveness of the tissue towards GA, possibly by decreasing the levels of ABA, a known antagonist of GA. The primary target tissue for GA action is the intercalary meristem of the internode (Sauter & Kende, 1992; Sauter et al., 1993). In recent years, much attention has been focused towards identifying novel genes that are part of the signal transduction pathway that leads to submergence-induced growth of internodes. Through subtractive hybridization techniques, several genes have been isolated from deepwater rice that are differentially expressed in the intercalary meristem in response to GA and that may play a role in GA-induced stem elongation. Examples include an ortholog of the replication protein A1 (van der Knaap et al., 1997), a leucine-rich repeat receptor like protein kinase (van der Knaap et al., 1999) and a novel gibberellin-induced gene termed Oryza sativa Growth Regulating Factor 1 (van der Knaap et al., 2000).

In contrast with internodal growth, the induction of adventitious root growth upon flooding is less well understood. Adventitious roots are shoot-borne roots that are initiated as part of normal plant development in deepwater rice. The formation of adventitious roots occurs in distinct developmental stages: (1) initiation, (2) early development, (3) growth arrest, and (4) emergence of the root primordium through the nodal meristem. Stages (1) through (3) are part of the normal plant development; as the plant develops, root initials mature to root primordia that bear all the characteristics of primary or lateral roots but then remain dormant. Step (4), i.e. emergence of the root primordia through the nodal meristem, is not part of normal plant development and needs to be triggered by the right stimulus such as submergence of the internode in water or by ethylene treatment.

Some stages of adventitious root development have been characterized at the molecular level on the basis of differential gene expression and for some stages mutant phenotypes are available. However, the physiological, biochemical and molecular processes that underlie adventitious root formation in plants are far from understood. Studies with plant hormones have shown that the growth of adventitious roots can be induced by treatment with ethylene but not by treatment with auxin, cytokinin or gibberellin. Therefore, in adventitious roots ethylene seems to be the hormonal signal that leads directly to meristem activation, as opposed to internodal growth, which is triggered by gibberellin. Therefore, specific signal transduction pathways must exist in these two organs with respect to ethylene response and growth induction. To date, no genes have been identified that are differentially expressed during submergence-induced growth of adventitious roots or that otherwise may be involved in this growth process.

The classical plant hormones such as auxins, cytokinins and others, do not have a peptide structure, in contrast with growth factors and hormones in bacteria and animals. Only recently peptide hormones have been identified In plants. These peptides regulate defence, fertilization, and also growth and development responses of the plants (Ryan & Pearce, 2001). Phytosulfokine-α (PSK-α) is a sulfated pentapeptide originally isolated from a plant cell culture medium. PSK-α promotes plant cell proliferation in in vitro cultures and, when supplemented to growth media, has various biological activities related to plant cell growth and differentiation (Yang et al., 2000). However, so far a role for PSK-α in growth processes in intact plants has remained elusive. The cDNA encoding PSK-α, OsPSK, has recently been isolated from rice (Yang et al., 1999; patent application No FR 2791347).

The number of genes or gene products that regulate plant growth responses is currently limited. Similarly, the number of compounds that can be used as exogenous plant growth regulators is also limited. It would be very desirable to have additional genes or substances for controlling or modifying the growth characteristics of a plant or of specific organs or tissues of a plant. The present invention provides compositions and methods for regulating plant growth processes. The compositions and methods have wide application in agricultural and horticultural practices and also in in vitro plant cell and tissue culture.

### SUMMARY OF THE INVENTION

The present invention provides methods for regulating the growth characteristics of a plant or of an organ or tissue or cell of the plant using DNA sequences encoding GREP growth regulating proteins and the corresponding GREP proteins.

The term “GREP” relates to said proteins (or genes encoding said proteins) that comprise the GREP signature motif.

In one aspect of the invention, the methods comprise the introduction and/or functional expression of one or more growth regulating proteins in a plant or in parts thereof and/or one or more DNA sequences encoding such proteins. In another aspect of the invention, the methods comprise the modification of functional expression of native growth regulating protein genes in plants or plant parts and the use of the growth regulating protein sequences as general molecular markers or for the selective breeding of growth regulating protein encoded traits in non-transgenic approaches for crop improvement. In another aspect of the invention, the methods comprise the use of formulations that contain growth regulating proteins or the active peptide(s) derived from said growth regulating proteins as plant growth regulators in applications related to agriculture, horticulture and in vitro plant cell and tissue culture.

The present invention also relates to DNA sequences encoding GREP growth regulating proteins, the corresponding amino acid sequences, and methods for obtaining the same. A peptide consensus sequence termed the GREP signature motif as well as the correlating nucleic acid sequence which encodes the GREP signature motif, are also provided.

Methods for the identification of compounds that interact with or are targeted by growth regulating proteins are also provided by the invention. The present invention further provides transgenic plant cells, plant tissues and plants containing growth regulating protein sequences and vectors. The present invention also provides vectors comprising said DNA sequences wherein the DNA sequences are operatively linked to regulatory elements allowing expression in prokaryotic and/or eukaryotic host cells.

In addition, the present invention relates to the proteins encoded by GREP encoding nucleic acid sequences, antibodies to the proteins and methods for their production. Furthermore, the present invention relates to regulatory sequences, which naturally regulate the expression of GREP encoding DNA sequences.

The present invention relates to a group of growth regulating proteins (GREP), polypeptides, or functional fragments thereof encoded by nucleic acids comprising a nucleotide sequence encoding an amino acid sequence (GREP signature motif) of the formula;

CX₁X₂X₃CX₄X₅X₆X₇HX₈DYIYTX₉ (SEQ ID NO 52)

wherein X₁ are 4 to 8 amino acids, X₂ is D or E, X₃ is one or two amino acids, X₄ are two or three amino acids, X₅ is R or K, X₆ is R or K, X₇ are 4 or 5 amino acids, X₈ is any amino acid and X₉ is Q or H.

The invention also relates to an isolated nucleic acid encoding a growth regulating polypeptide (GREP) comprising an amino acid sequence which is at least 90% identical preferably at least 90.5%, 91%, 91.5%, 92%, 92.5%, 93%, 93.5%, 94%, 94.5%, 95% identical, more preferably at least 95.5%, 96%, 96.5%, 97%, 97.5%, 98%, 98.5% identical, most preferably 99% or 99.5% identical to the sequence as represented in SEQ ID NO 52, or a functional fragment of such a GREP protein or polypeptide.

The term “polypeptide” as used herein also means protein or peptide and is used interchangeable throughout the description.

It is to be understood that the expression “functional fragment thereof” relates to fragments of said growth regulating proteins which have a similar biologic activity as the GREP, preferably said functional fragments comprise the GREP signature motif. Instead of “functional fragment” also the expression “bioactive peptide” may be used herein.

The GREP signature motif is herein identified as an amino acid sequence which is at least 90% identical, preferably at least 90.5%, 91%, 91.5%, 92%, 92.5%, 93%, 93.5%, 94%, 94.5%, 95% identical, more preferably at least 95.5%, 96%, 96.5%, 97%, 97.5%, 98%, 98.5% identical, most preferably 99% or 99.5% identical to the sequence represented by SEQ ID NO 52.

The invention furhter relates to the isolated nucleic acids encoding proteins, polypeptides or functional fragment thereof comprising the GREP signature motif.

Examples of such proteins, polypeptides, fragments thereof and nucleic acids encoding the same are provided and represented in any of SEQ ID NOs 1 to 103.

The inventors thus now found and characterized a new and large family of GREP growth regulating proteins, all containing the GREP signature motif. These family members can be identified in a whole range of plant species, and therefor, all plant GREP genes and proteins can be used in the methods of the present invention.

Until now only one other growth regulating protein (OsPSK) was identified that is very closely related but that does not contain the complete GREP motif. However, it has been shown by the inventors that OsPSK is useful in similar applications as for the GREP growth regulating proteins. The DNA sequence of OsPSK is represented in SEQ ID NO 104 and the corresponding amino acid sequence is represented in SEQ ID NO 105.

According to an interesting embodiment of the invention, there is provided an isolated nucleic acid molecule encoding a protein, or a functional fragment thereof, comprising an amino acid sequence as set forth in any of SEQ ID NOs 2, 4, 6, 9, 12, 15, 17, 20, 23, 26, 29, 31, 33, 35, 37, 39, 41, 43, 45, 47, 49, 51, 52, 55, 57, 59, 61, 63, 65, 67, 70, 73, 75, 77, 79, 81, 83, 85, 87, 89, 91, 93, 95, 97, 99, 101 or 103. Such an isolated nucleic acid molecule may comprise for instance the nucleotide sequence as set forth in any of SEQ ID NOs 1, 3, 5, 7, 8, 10, 11, 13, 14, 16, 18, 19, 22, 24, 25, 27, 28, 30, 32, 34, 36, 38, 40, 42, 44, 46, 48, 50, 53, 54, 56, 58, 60, 62, 64, 66, 68, 69, 71, 72, 74, 76, 78, 80, 82, 84, 86, 88, 90, 92, 94, 96, 98, 100 or 102.

The present invention also provides an isolated nucleic acid molecule encoding a protein having an amino acid sequence as set forth in any of SEQ ID NOs 2, 12, 70 or 73. Such an isolated nucleic acid molecule may comprise a nucleotide sequence as set forth in e.g., in any of SEQ ID NOs 1, 10, 11, 68, 69, 71 or 72, respectively.

In addition, the present invention provides an isolated nucleic acid molecule consisting of a nucleotide sequence encoding an amino acid sequence (GREP signature motif) of the formula:

CX₁X₂X₃CX₄X₅X₆X₇HX₈DYIYTX₉ (SEQ ID NO 52)

wherein X₁ are 4 to 8 amino acids, X₂ is D or E, X₃ is one or two amino acids, X₄ are two or three amino acids, X₅ is R or K, X₆ is R or K, X₇ are 4 or 5 amino acids, X₈ is any amino acid and X₉ is Q or H,

or encoding an amino acid sequence which is at least 90% identical, preferably at least 90.5%, 91%, 91.5%, 92%, 92.5%, 93%, 93.5%, 94%, 94.5%, 95% identical, more preferably at least 95.5%, 96%, 96.5%, 97%, 97.5%, 98%, 98.5% identical, most preferably 99% or 99.5% identical to the sequence as represented in SEQ ID NO 52.

Such an isolated nucleic acid molecule encoding the GREP signature motif may consist of the formula:

TGYN₁GAN₂TGYN₃MRNMRN₄CAYNNNGAYTAYATHTAYACNCAN (SEQ ID NO 53)

wherein M is A or C, R is A or G, Y is C or T, H is A or C or T, and N is G or A or T or C, and wherein N₁ is a stretch of 12 to 24 amino acid residues, N₂ is a stretch of 4 to 7 amino acid residues, N₃ is a stretch of 6 to 9 amino acid residues and N₄ is a stretch of 13 to 16 amino acid residues.

The present invention further relates to any protein, polypeptide or peptide encoded by any of the nucleic acids described herein.

In another embodiment of the invention, there is provided a vector comprising a nucleotide sequence encoding a plant GREP growth regulating protein, wherein the GREP growth regulating protein comprises an amino acid sequence of the formula:

CX₁X₂X₃CX₄X₅X₆X₇HX₈DYIYTX₉ (SEQ ID NO 52)

wherein X₁ are 4 to 8 amino acids, X₂ is D or E, X₃ is one or two amino acids, X₄ are two or three amino acids, X₅ is R or K, X₆ is R or K, X₇ are 4 or 5 amino acids, X₈ is any amino acid and X₉ is Q or H,

or a vector comprising an isolated nucleic acid encoding a GREP growth regulating polypeptide comprising an amino acid sequence which is at least 90% identical, preferably at least 90.5%, 91%, 91.5%, 92%, 92.5%, 93%, 93.5%, 94%, 94.5%, 95% identical, more preferably at least 95.5%, 96%, 96.5%, 97%, 97.5%, 98%, 98.5% identical, most preferably 99% or 99.5% identical to the sequence as represented in SEQ ID NO 52, or a functional fragment of such a GREP growth regulating protein or polypeptide.

Such a vector may comprise a nucleotide sequence having the formula:

TGYN₁GAN₂TGYN₃MRNMRN₄CAYNNNGAYTAYATHTAYACNCAN (SEQ ID NO 53)

wherein M is A or C, R is A or G, Y is C or T, H is A or C or T, and N is G or A or T or C, and wherein N₁ is a stretch of 12 to 24 amino acid residues, N₂ is a stretch of 4 to 7 amino acid residues, N₃ is a stretch of 6 to 9 amino acid residues and N₄ is a stretch of 13 to 16 amino acid residues.

A vector of the present invention may comprise a nucleotide sequence for a GREP growth regulating protein having a molecular weight in the range of from about 7 kD to about 13 kD or may encode a fragment thereof. The GREP growth regulating protein encoded by a nucleotide sequence of such a vector may comprise a hydrophobic N-terminal leader sequence. The amino acid sequence set forth in SEQ ID NO 52 is preferably located near the carboxy-terminus of the GREP growth regulating protein. The nucleotide sequence of a subject vector preferably encodes the amino acid sequence set forth in SEQ ID NO 52 (GREP signature motif). In a vector comprising the nucleotide sequence for a GREP fragment or a full length GREP, the GREP signature motif is preferably located near the carboxy-terminus of the GREP growth regulating protein. Nucleotide sequence encoding the GREP signature motif in a subject vector may be preceded by an acidic region and/or followed by a basic region. In a vector having coding sequence for a full length or near full-length GREP growth regulating protein, the sequence may encode a protein having three alpha-helix structures in the post leader sequence.

The invention further relates to a vector comprising a nucleic acid encoding any of the GREP growth regulating polypeptides as described herein, or a vector comprising a nucleic acid encoding the rice growth regulating polypeptide OsPSK as represented in SEQ ID NO 105 wherein said growth regulating proteins regulate growth and/or development response in intact plants.

A subject vector may be an expression vector wherein the nucleotide sequence encoding any of the GREP growth regulating polypeptides as described herein, or a vector comprising a nucleic acid encoding the rice growth regulating polypeptide OsPSK as represented in SEQ ID NO 105, is under the control of a promoter which functions in plants. The promoter may be a tissue-preferred or tissue-specific promoter, for example a seed specific promoter such as the 2S2 promoter or the prolamin, oleosin or beta-expansine promoter, or a meristem specific promoter such as the cdc2a or the RNR1 promoter, or a root specific promoter, such as the lipase, metallothionein or RCH1 promoter. The promoter may also be an inducible promoter or constitutive promoter, such as the ubiquitin, CaMV 35S or pGOS2 promoter.

In a particular embodiment, the present invention relates to a vector comprising a nucleic acid encoding a GREP growth regulating polypeptide as defined in any of claims 4 to 6 or a vector comprising a nucleic acid encoding the rice growth regulating polypeptide OsPSK as represented in SEQ ID NO 105 wherein said growth regulating proteins regulate growth and/or development response in intact plants and wherein the genes encoding said growth regulating proteins are under the control of a ubiquitin promoter.

In a more particular embodiment, said ubiquitin promoter is the sunflower ubiquitin promoter. In a more specific embodiment this vector is similar to the p2743 vector or the p0531 vector as described in Example 12 or in FIG. 13 or 21 respectively.

Accordingly, in a related embodiment, the present invention relates to a transgenic plant transformed with a vector as described above.

A subject vector may also comprise a terminator. The GREP growth regulating protein-genes may be or may comprise cDNA or genomic DNA. GREP encoding sequences may also be synthetic. Thus for example, a vector of the present invention may comprise a sequence such as represented in any of SEQ ID NOs 1, 3, 5, 7, 8, 10, 11, 13, 14, 16, 18, 19, 22, 24, 25, 27, 28, 30, 32, 34, 36, 38, 40, 42, 44, 46, 48, 50, 53, 54, 56, 58, 60, 62, 64, 66, 68, 69, 71, 72, 74, 76, 78, 80, 82, 84, 86, 88, 90, 92, 94, 96, 98, 100, 102 and/or 104.

Table 4 lists each of the foregoing sequence identifiers and indicates the source and type of each sequence. Prefixes to GREP or PSK sequences under “Name” indicate sequence source. Ao, Asparagus officinalis; At, Arabidopsis thaliana; Bn, Brassica napus; Ga, Gossypium arboreum; Gm, Glycine max; Le, Lycopersicon esculentum; Mc, Mesembryanthemum cristallinum; Os, Oryza sativa; Pt, Pinus taeda; Sb, Sorghum bicolor; Sp, Sorghum propinquum; St, Solanum tuberosum; Ta, Triticum aestivum; Zm, Zea mays. In literature, some of these sequences have been identified as belonging to the group of “PSK” sequences. Therefore, alternative names are provided in separate columns for ease of comparison with published articles.

Also in accordance with the present invention, there are provided vectors in which the nucleic acid encoding any of the GREP growth regulating polypeptides as described herein, or the nucleic acid encoding the rice growth regulating polypeptide OsPSK as represented in SEQ ID NO 105, is in a sense or antisense orientation relative to the promoter sequence. If desired, for co-suppression or antisense applications, a complete gene/cDNA/ORF or partial sequence which does not encode a functional protein may be used.

The present invention also provides a transgenic plant, an essentially derived variety thereof, plant part, plant cell, or protoplast which comprises a nucleic acid encoding any of the GREP growth regulating polypeptides as described herein, or the nucleic acid encoding the rice growth regulating polypeptide OsPSK as represented in SEQ ID NO 105, wherein said nucleotide sequence is heterologous to the genome of said transgenic plant, essentially derived variety thereof, plant part, plant cell or plant protoplast.

In another embodiment of the invention, there is provided a plant, essentially derived variety thereof, plant part, plant cell or protoplast wherein the plant, essentially derived variety thereof, plant part, plant cell, or protoplast has been transformed with a nucleotide sequence encoding any of the GREP growth regulating proteins of the invention or which has been transformed with a nucleic acid encoding the rice growth regulating polypeptide OsPSK as represented in SEQ ID NO 105.

The present invention also provides a plant, essentially derived variety thereof, plant part, plant cell, or protoplast which overexpresses any of the GREP growth regulating proteins of the invention or which overexpresses the rice growth regulating polypeptide OsPSK as represented in SEQ ID NO 105.

Transformation may be transient or stable. The invention thus also relates to such a stably or transiently transformed transgenic plant or plant cell. The invention further relates to any plant which comprises any of the subject vectors in accordance with the invention.

According to a further embodiment, the invention also relates to any of the transgenic plants described herein comprising a nucleic acid encoding any of the GREP growth regulating polypeptides as described herein, or comprising the nucleic acid encoding the rice growth regulating polypeptide OsPSK as represented in SEQ ID NO 105, characterized in that said plant has altered growth and/or yield and/or development characteristics, for instance increased inflorescence, for instance increased inflorescence of 30% to 70%. For instance, also according to the present invention, in said plants, the ratio between the size of inflorescence before harvest and the maximal measured size of the leaf rosette is increased.

The invention further relates to transgenic plants as described above characterized in that said plant has larger seeds or shows early vigour, or shows increased cell proliferation in early seed development.

Seed from a subject transgenic plant or essentially derived variety thereof is also provided as are pollen, harvestable parts or propagation material including, e.g., a flower, a seed, a cutting, a root, a tuber, or an explant.

The present invention also provides host cells which comprise a nucleic acid encoding any of the GREP growth regulating proteins as described herein, wherein the nucleic acid is heterologous to the genome of the host cells or wherein the host cells have been transfected or transformed with a nucleic acid encoding a GREP growth regulating protein. Examples of host cells which may be used in accordance with the present invention include bacterial, yeast, fungal, insect, mammalian or plant cell. Preferably, plant cells may be used.

The host cells in accordance with the present invention may comprise a nucleic acid encoding any of the GREP growth regulating proteins as described herein or encoding the rice growth regulating polypeptide OsPSK as represented in SEQ ID NO 105, in a sense or antisense orientation relative to a regulatory region directing expression of said nucleic acid, said nucleic acid may also be included in a gene silencing construct driven by a regulatory region.

In a further aspect of the invention, there is provided an isolated antisense molecule consisting of from about 14 to about 100 nucleotides targeted to the nucleotide sequence of SEQ ID NO 53, preferably said molecule consists of 20, 30, 40, 50, 60, 70, 80 or 90 nucleotides.

An antibody which recognizes and binds to a plant GREP growth regulating protein or a fragment thereof is also provided. The antibody may be a monoclonal or polyclonal antibody. In an interesting embodiment, the GREP fragment to which the antibody binds comprises an amino acid sequence which is at least 90% identical, preferably at least 90.5%, 91%, 91.5%, 92%, 92.5%, 93%, 93.5%, 94%, 94.5%, 95% identical, more preferably at least 95.5%, 96%, 96.5%, 97%, 97.5%, 98%, 98.5% identical, most preferably 99%-or 99.5% identical to the sequence as represented in SEQ ID NO 52.

Also provided by the present invention are methods for altering growth and/or development of a plant or plant cell which comprises modulating the level and/or activity of any of the GREP growth regulating protein as herein described or modulating the level and/or activity of the rice growth regulating polypeptide OsPSK as represented in SEQ ID NO 105 in the plant or plant cell. Said methods include the introduction of heterologous GREP or OsPSK genes in a plant or plant cell via transformation. Modulation of the level and/or activity of an endogenous GREP or OsPSK growth regulating polypeptide may be achieved using such methods as e.g., targeted mutation of endogenous GREP or OsPSK growth regulating genes or polypeptides or their regulatory sequences, or juxtapositioning regulatory sequences such as enhancers in the region of a nucleotide sequence coding for a GREP or an OsPSK growth regulating polypeptides. Thus, by such a method, the level and/or activity of a GREP or an OsPSK growth regulating gene or polypeptide may be increased or decreased. The level and/or activity of a GREP or OsPSK growth regulating polypeptide may be increased by, e.g., increasing transcription of a nucleotide sequence encoding the GREP or OsPSK growth regulating polypeptide.

The genes according to the present invention can also be used to produce transgenic plants with altered growth characteristics. These applications are also useful for the OsPSK growth regulating protein, which is closely related to the growth regulating proteins of the present invention, but which do not contain the GREP motif.

In a particular embodiment the present invention relates to a method for altering growth and/or development of a plant storage organ or part thereof which comprises modulating the level and/or activity of any of the growth regulating polypeptide as defined herein or modulating the level and/or activity of the rice growth regulating polypeptide OsPSK is as represented in SEQ ID NO 105 in the meristem or part thereof.

In another aspect of the invention, there is provided a method for altering growth and/or development of a plant storage organ or part thereof which comprises modulating the level and/or activity of a GREP growth regulating protein or modulating the level and/or activity of the rice growth regulating polypeptide OsPSK as represented in SEQ ID NO 105 in the storage organ or part thereof. The storage organ or part thereof may be e.g., a seed, root, tuber, or fruit. Thus, by such method, the level and/or activity of a GREP or OsPSK growth regulating protein may be increased or decreased in the storage organ or in a part thereof. The level and/or activity of a GREP or OsPSK growth regulating protein may be increased by, e.g., increasing transcription of a nucleotide sequence encoding the GREP or OsPSK growth regulating protein in the storage organ or in a part thererof.

Modulation of the level or activity of a GREP or OsPSK growth regulating protein in a plant or plant cell, or in a storage organ or in a part of said plant, plant cell or storage organ may be by administering or exposing the plant or plant cells to a GREP or OsPSK growth regulating polypeptide, a homologue of a GREP or OsPSK growth regulating polypeptide, an analogue of a GREP or OsPSK growth regulating polypeptide, a derivative of a GREP or OsPSK growth regulating polypeptide, and/or to an immunologically active fragment of a GREP or OsPSK growth regulating polypeptide.

In another aspect of the invention, there is provided a method of downregulating levels of any of the GREP growth regulating protein gene products as described herein, or down-regulating levels of the rice growth regulating polypeptide OsPSK gene product as represented in SEQ ID NO 105, or downregulating GREP or OsPSK gene product activity which comprises administration of GREP or OsPSK antibodies to cells, tissues, or organs of a plant or exposing cells, tissues, or organs of a plant to GREP or OsPSK antibodies, respectively.

In still another aspect of the invention, there is provided a method of downregulating levels of any of the GREP (growth regulating protein) gene products or downregulating levels of the rice growth regulating polypeptide OsPSK gene product as represented in SEQ ID NO 105, or downregulating GREP or OsPSK gene product activity which comprises expressing antibodies to the GREP or OsPSK gene product in a cell, tissue or organ of a plant, respectively.

The present invention also provides a method of regulating growth and/or development of a plant or cell, tissue or organ of a plant which comprises contacting the cell, tissue, or organ of the plant with a plant GREP growth regulating protein or the bioactive peptide derived from a GREP growth regulating protein, or comprises contacting the cell, tissue, or organ of the plant with the rice growth regulating protein OsPSK as represented in SEQ ID NO 105. The bioactive peptide (or a functional fragment) derived from the GREP growth regulating protein may also be used in such a method. Further, in this method, the GREP growth regulating protein or a bioactive peptide derived from a GREP growth regulating protein may be added to the growth media of the plant. Alternatively, the GREP growth regulating protein or functional fragment or bioactive peptide derived therefrom may be applied directly to the plant or a part thereof as part of a formulation in a liquid or solid composition. Examples of GREP and OsPSK growth regulating polypeptides which may be used in the methods of the invention include (but are not limited to) polypeptides comprising or consisting of any of the amino acid sequences as set forth in SEQ ID NOs 2, 4, 6, 9, 12, 15, 17, 20, 23, 26, 29, 31, 33, 35, 37, 39, 41, 43, 45, 47, 49, 51, 52, 55, 57, 59, 61, 63, 65, 67, 70, 73, 75, 77, 79, 81, 83, 85, 87, 89, 91, 93, 95, 97, 99, 101, 103 or 105.

Table 4 lists each of the foregoing sequence identifiers and indicates the source and type of each sequence. Prefixes to GREP or PSK sequences under “Name” indicate sequence source. Ao, Asparagus officinalis; At, Arabidopsis thaliana; Bn, Brassica napus; Ga, Gossypium arboreum; Gm, Glycine max; Le, Lycopersicon esculentum; Mc, Mesembryanthemum cnstallinum; Os, Oryza sativa; Pt, Pinus taeda; Sb, Sorghum bicolor; Sp, Sorghum propinquum; St, Solanum tuberosum; Ta, Triticum aestivum; Zm, Zea mays. In literature, some of these sequences have been identified as belonging to the group of UPSI sequences. Therefore, alternative names are provided in separate columns for ease of comparison with published articles.

The present invention also provides a peptide consisting of the amino acid sequence of the formula

CX₁X₂X₃CX₄X₅X₆X₇HX₈DYIYTX₉ (SEQ ID NO 52)

wherein X₁ are 4 to 8 amino acids, X₂ is D or E, X₃ is one or two amino acids, X₄ are two or three amino acids, X₅ is R or K, X₆ is R or K, X₇ are 4 or 5 amino acids, X₈ is any amino acid and X₉ is Q or H,

or consisting of an amino acid sequence which is at least 90% identical, preferably at least 90.5%, 91%, 91.5%, 92%, 92.5%, 93%, 93.5%, 94%, 94.5%, 95% identical, more preferably at least 95.5%, 96%, 96.5%, 97%, 97.5%, 98%, 98.5% identical, most preferably 99% or 99.5% identical to the sequence as represented in SEQ ID NO 52.

The present invention also provides a method for identifying alleles of GREP growth regulating proteins and selecting alleles with desired features. In one embodiment, the method comprises using GREP sequences or parts of GREP sequences for isolating GREP alleles and testing their features by expression in transgenic plants.

Alternatively, sequences located on the genome in the neighbourhood of GREPs may be used as molecular markers for different GREP alleles and specific GREP alleles may be selected by marker-assisted breeding. Such molecular markers are useful for plant breeding programs and selecting alleles with desired features. In one embodiment, the method comprises using GREP sequences or parts of GREP sequences for isolating GREP alleles and testing their features by expression in transgenic plants.

### BRIEF DESCRIPTION OF THE DRAWINGS

FIG. 1a shows the OsGREP1 cDNA sequence and deduced protein sequence. The CTC repeat in the 5′UTR is underlined. Stop codons in the 5′UTR preceding the start codon are in bold and italic. An arrowhead indicates the cleavage site of the putative signal peptide. The start and stop codon are indicated in bold.

FIG. 1b is a Hydropathy plot based on the method by (Kyte & Doolittle, 1982). Positive numbers indicate hydrophobic polypeptide regions. The N-terminal putative signal peptide is indicated as well as the acidic domain.

FIG. 1c is a secondary structure analysis according to (Stultz et al., 1993). The probability for an α-helical structure is given as a line. Such probability is nearly 1 for the signal peptide region and three additional regions in the post-leader sequence. The probability for a turn is indicated by a shaded curve. The highest probability for a turn exists around position 70 between helix 1 and 2 of the post-leader sequence. The signal peptide and acidic region are indicated as in FIG. 1b.

FIG. 2 is a photographic representation of a Northern blot showing OsGREP1 mRNA level in adventitious roots and in internode tissues of submerged and non-submerged deepwater rice plants. In adventitious roots, gene expression was analyzed at 0, 2 and 6 h after submergence. In the internode tissues, gene expression was analyzed at 0, 2, 6, and 18 h after submergence. The intercalary meristem (IM), the cell elongation zone (Ez) and cell differentiation zone (DZ) were analyzed separately. OsGREP1 is expressed in unsubmerged roots and further induced upon submergence in all three zones of the internode with maximal induction in EZ and IM. Ethidium bromide-stained ribosomal RNA indicates loading of the gel.

FIG. 3 is an alignment of full-length GREP peptide sequences and OsPSK generated with ClustalX 1.81 and with minor manual alignment. Horizontal lines above the alignment indicate the putative signal peptide and the conserved acidic and basic region. Conserved amino acids are shown with a background: black, 100% conserved; dark grey, at least 70% conserved; light grey, at least 50% conserved. The GREP signature motif is indicated below the alignment.

FIG. 4 is a statistics report calculated with GeneDoc 2.1 based on the alignment shown in

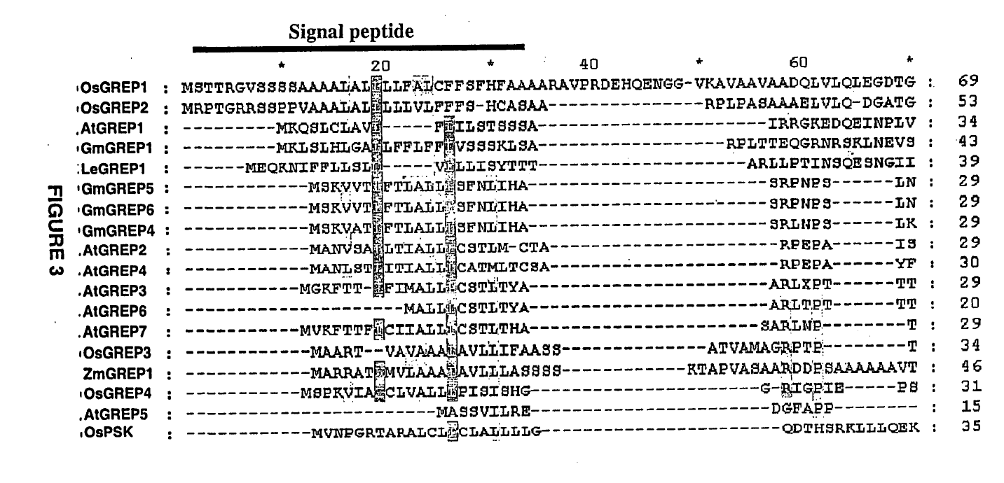

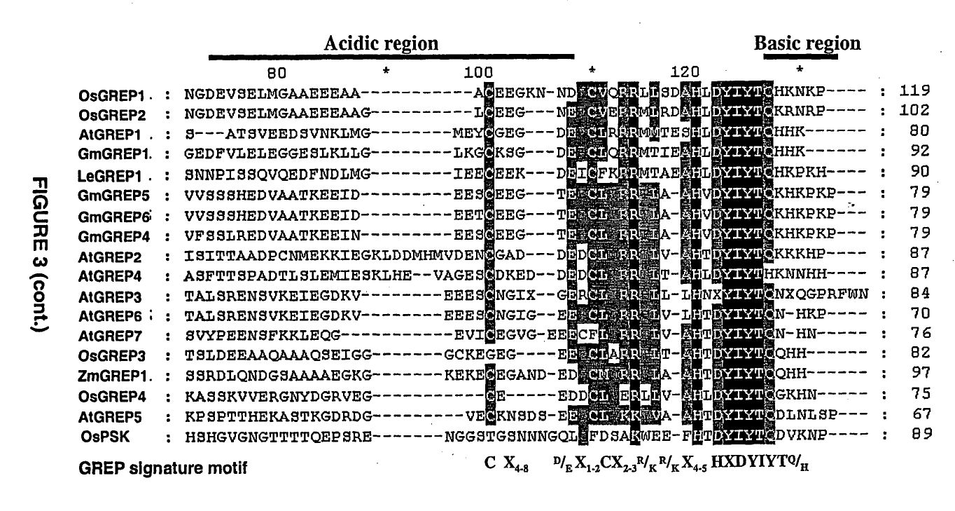

FIG. 3. Numbers above the diagonal refer to identical residues (in percentages), and numbers below the diagonal to similar residues (in percentages).

FIG. 5 is a phylogenetic tree of all GREP growth regulating protein sequences (including partial proteins) calculated with the ClustalX 1.81 program and displayed with TreeView 1.5.2. OsPSK was defined as outgroup and the tree was rooted with the outgroup. Scale bar the bar of 0.1 indicates 0.1 amino acids substitutions per site.

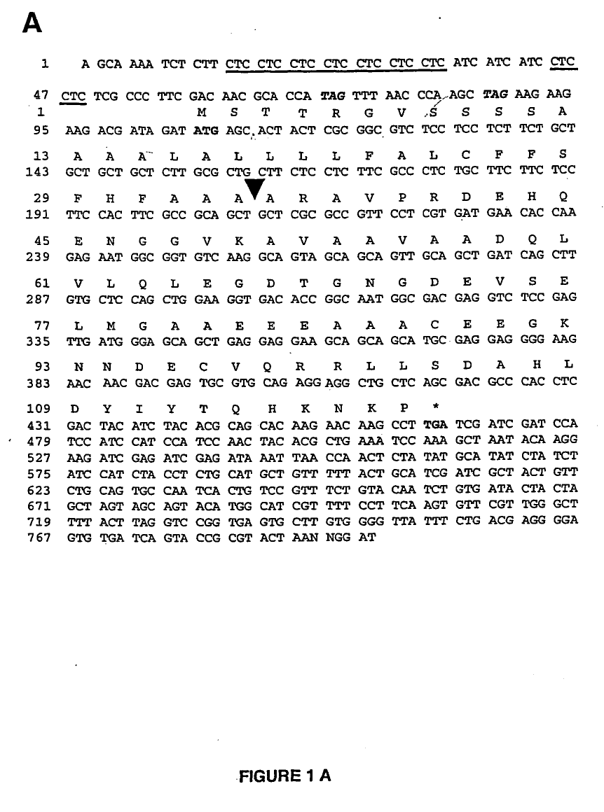

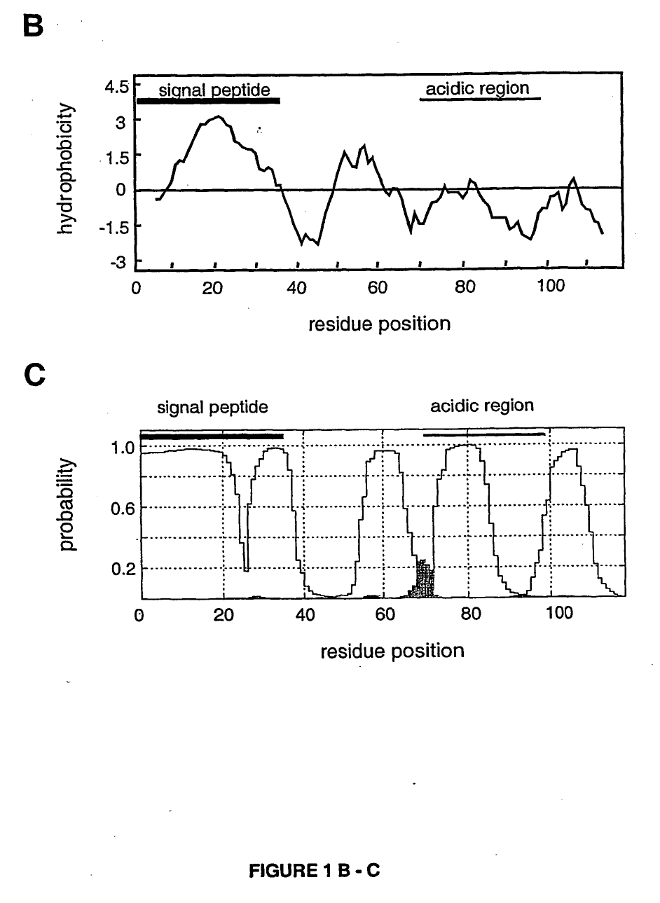

FIGS. 6a through 6h show secondary structure analyses of the GREP polypeptides (FIGS. 6b-6h) and the OsPSK protein (FIG. 6a) according to (Stultz et al., 1993). The probability for an α-helical arrangement of the protein sequences is indicated as a line. The probability for a turn is indicated as a shaded area. The GREP growth regulating proteins and the protein encoded by OsPSK have conserved structural features including three α-helices in the post-leader sequence and a turn between helix 1 and 2 of the post-leader sequence, similar to OsGREP1 shown in FIG. 1c.

FIG. 7 is a photographic representation of a Northern blot showing OsGREP1 mRNA expression in different tissues of adult deepwater rice plants or seedlings and in suspension-cultured rice cells. OsGREP1 mRNA levels are highest in root tissues and the coleoptile of seedlings, and lower in rice suspension cells. RNA loading is indicated as ethidium bromide-stained ribosomal RNA.

FIG. 8 is a photographic representation of a Northern blot showing mRNA level of OsGREP1 in stem sections of deepwater rice treated with gibberellin (GA) for the times indicated in hours (h) and analyzed in the intercalary meristem (IM) and in the elongation zone (EZ). The OsGREP1 mRNA level is induced at 0.5 and again at 15 h after GA treatment. Ethidium bromide staining of ribosomal RNA gives was used as an indication for total RNA loading.

FIG. 9 is a photographic representation of a Northern blot showing OsGREP1 transcripts in stem sections of deepwater rice treated with cycloheximide at the concentrations indicated (0 to 20 μg/ml). OsGREP1 transcripts accumulate in the presence of cycloheximide at concentrations of 0.2 μg/ml or higher. Ethidium bromide staining of ribosomal RNA was used as an indication for total RNA loading.

FIG. 10 is a plasmid map of the vector p0385. This circular vector contains 2744 base pairs and multiple restriction sites indicated by the italicised names. Between brackets, the place of the restriction site is indicated ori is the origin of replication; T1 and T2 are terminator sites; attL1 and attL2 are the attachment sites L1 and L2 of the Gateway recombination cassette; codB is a ccdB resistance gene; KMr is the kanamycin resistance gene.

FIG. 11 is a plasmid map of the vector p0403. This circular vector contains 2546 base pairs and multiple restriction sites indicated by the italicised names. Between brackets, the place of the restriction site is indicated ori is the origin of replication; T1 and T2 are terminator sites; attL1 and attL2 are the attachment sites L1 and L2 of the Gateway recombination site; SENSE PRM is the sense primer; ANTISENSE PRM is the antisense primer; PSK is the gene encoding phytosulphokine of Oryza sativa (OsPSK); KMr is the kanamycin resistance gene.

FIG. 12 is a plasmid map of the vector p0712. This circular vector contains 11206 base pairs. pBR322 (ori+bom) is the origin of replication and the bom site of the plasmid pBR322; Sm/SpR is streptomycin/spectinomycin resistance gene, LB Ti C58 and RB Ti C58 are respectively the left and right border regions of the Ti plasmid C58; LB repeat nopaline and RB repeat nopaline are respectively the left and right core repeats of the left and right border regions; pNOS is the promoter sequence of the nopaline synthase gene; tOCS is the terminator sequence of the octopine synthase; tNOS is the terminator sequence of the nopaline synthase gene; pUBI is the promoter of the sunflower ubiquitin gene; attR1 and attR2 are the attachment sites R1 and R2 of the Gateway recombination cassette respectively; CamR is the Chloramphenicol resistance gene; ccdB is the ccdb resistance gene, T zein is the terminator of zein; T-rbcS-deltaGA the terminator of the pea ribusco gene of which a G and A were deleted.

FIG. 13 is a plasmid map of the vector p2743. This circular vector contains 9868 base pairs. pBR322 (ori+bom) is the origin of replication and the bom site of the plasmid pBR322; Sm/SpR is is streptomycin/spectinomycin resistance gene; LB Ti C58 and RB Ti C58 are respectively the left and right border regions of the Ti plasmid C58; LB repeat nopaline and RB repeat nopaline are respectively the left and right core repeats of the left and right border regions; pNOS is the promoter sequence of the nopaline synthase gene; tOCS is the terminator sequence of the octopine synthase; tNOS is the terminator sequence of the nopaline synthase gene; pUBI the promoter of the sunflower ubiquitin gene; PSK is the gene encoding phytosulphokine from Oryza sativa (OsPSK; T zein is the terminator of zein; T-rbcS-deltaGA the terminator of the pea ribusco gene of which a G and A were deleted.

FIG. 14 is a graphical analysis of the rosette size in function of the time. The X-axis shows the time in days (with 0 as the day of sowing). The Y-axis shows the surface of the rosettes of each plant in cm². The OsPSK transgenic plant is indicated as θ. The other transgenic plants (named AE0017, AE0018, AE0018, AE0019, AE0021, AE0022, AE0023, AE0024, AE0025, AE0026, AE0027) are indicated with the symbol ▪. Error bars are standard errors.

FIG. 15 is a graphical analysis of the inflorescence size in function of the time. The X-axis shows the time in days (with 0 as the day of sowing). The Y-axis shows the surface of the inflorescence of each plant in cm². The OsPSK transgenic plant is indicated as θ. The other transgenic plants (named AE0017, AE0018, AE0018, AE0019, AE0021, AE0022, AE0023, AE0024, AE0025, AE0026, AE0027) are indicated with the symbol □. Error bars are standard errors.

FIG. 16 is a graphical analysis of the ratio between inflorescence and rosette is shown for every transgenic plant line involved in the experiment (line ID on the X-axis). The Y-axis shows the ratio between inflorescence and rosette size in the different transgenic plant lines. Error bars are standard errors.

FIG. 17 is a digitalized picture of the different transgenic plant lines used in the phenotypic characterization experiments. All photographs are taken from the same distance and under the same conditions. The photographs show clearly that the inflorescence of the OsPSK transgenic plant line is greater than the inflorescence of the other transgenic plant lines.

FIG. 18 is an alignment of the protein sequences with the GREP motif. In this figure the PSK nomenclature as will be given in the future is used. Table 4 can be used as conversion table for the GREP nomenclature and the SEQ ID numbering. This figure is an alignment of full-length and partial GREP peptide sequences and OsPSK generated with the program ClustalX 1.81 and with minor manual alignment. Horizontal lines above the alignment indicate the putative signal peptide and the conserved acedic and basic region. The GREP motif is indicated below the alignment and the conserved amino acids corresponding to this GREP motif are shown with a gray background. The pentapeptide YIYTQ is boxed.

FIG. 19 is an alignment of OsGREP5 and OsGREP6 with the GREP motif. Alignment of SEQ ID NO 70 (OsGREP5) and SEQ ID NO 73 (OsGREP6) to demonstrate the presence of the GREP motif. The GREP motif is indicated below the alignment and the conserved amino acids corresponding to this GREP motif are shown with a grey background.

FIG. 20 is a plasmid map of the vector p0427. This circular vector contains 9914 base pairs. pBR322 (ori+bom) is the origin of replication and the bom site of the plasmid pBR322; Sm/SpR is streptomycin/spectinomycin resistance gene, LB Ti C58 and RB Ti C58 are respectively the left and right border regions of the Ti plasmid C58; LB repeat nopaline and RB repeat nopaline are respectively the left and right core repeats of the left and right border regions; pNOS is the promoter sequence of the nopaline synthase gene; tOCS is the terminator sequence of the octopine synthase; tNOS is the terminator sequence of the nopaline synthase gene; pUBI is the promoter of the sunflower ubiquitin gene; attR1 and attR2 are the attachment sites R1 and R2 of the Gateway recombination cassette respectively; CamR is the chloramphenicol resistance gene; ccdB is the ccdb resistance gene, the terminator used in this construct is the bidirect terminator of Agrobacterium.

FIG. 21 is a plasmid map of the vector p0531. This circular vector contains 8576 base pairs. pBR322 (ori+bom) is the origin of replication and the born site of the plasmid pBR322; Sm/SpR is is streptomycin/spectinomycin resistance gene; LB Ti C58 and RB Ti C58 are respectively the left and right border regions of the Ti plasmid C58; LB repeat nopaline and RB repeat nopaline are respectively the left and right core repeats of the left and right border regions; pNOS is the promoter sequence of the nopaline synthase gene; tOCS is the terminator sequence of the octopine synthase; tNOS is the terminator sequence of the nopaline synthase gene; pUBI the promoter of the sunflower ubiquitin gene; CDS0021 (PSK)-ATG is the the gene encoding phytosulphokine from Oryza sativa (OsPSK); the terminator used in this construct is the bidirect terminator of Agrobacterium.

FIG. 22 is a listing of all SEQ ID NOs and the corresponding nucleotide or protein sequences. For some alternative sequences (e.g. SEQ ID NOs 55 and 57 also an comparative sequence alignment (with SEQ ID NOs 2 and 4 respectively) is shown. In the genomic sequences the introns are underlined. The start and stop codons are in bold. For some sequences the GREP name as given by the inventors is indicated between brackets as well as the PSK nomenclature that corresponds to the nomenclature that is to be given in scientific literature. Abbreviations of plant species are as follows: Ao, Asparagus officinalis; At, Arabidopsis thaliana; Bn, Brassica napus; Ga, Gossypium arboreum; Gm, Glycine max, Le, Lycopersicon esculentum; Mc, Mesembryanthemum cristallinum; Os, Oryza sativa; Pt, Pinus taeda; Sb, Sorghum bicolor; Sp, Sorghum propinquum; St, Solanum tuberosum; Ta, Triticum aestivurm; Zm, Zea mays.

### DETAILED DESCRIPTION OF THE INVENTION

The present invention provides a differentially expressed cDNA isolated from growth-induced adventitious roots of deepwater rice and called OsGREP1 for Oryza sativa Growth Regulating Protein 1. Database searches using OsGREP1 resulted in the identification of gene families in rice, maize, Arabidopsis, soybean, rape and tomato encoding homologous proteins termed growth regulating proteins or GREPs comprising a GREP motif. Overall sequence identity of GREP growth regulating proteins at the protein level is usually low, averaging 15 to 35% except for a few analogues from the same species which have up to 98% sequence identity. Despite the low primary sequence conservation, certain primary and secondary structure characteristics for the GREP growth regulating proteins confirm their relationship. GREP growth regulating proteins are small proteins with a calculated molecular weight of 7 to 13 kD. GREP growth regulating proteins contain a hydrophobic N-terminal leader sequence that may function for targeting the GREP growth regulating proteins to the secretory pathway. Importantly, a new peptide signature pattern termed the GREP signature motif, has been identified. The GREP signature motif CX₄₋₈^{D}/_{E}X₁₋₂CX₂₋₃^{R}/_{K}^{R}/_{K}X₄₋₅HXDYIYT^{Q}/_{H} is located at the carboxyterminus of GREP growth regulating proteins. The OsPSK protein described by Yang et al. (1999) shares some of the characteristics of GREP growth regulating proteins. Notably, OsPSK and GREP growth regulating proteins share the YIYT sequence, which is part of the GREP signature motif and also corresponds to part of the pentapeptide backbone YIYTO of the plant growth regulator PSK-α. However, the protein encoded by OsPSK does not contain the complete GREP signature motif and the overall peptide sequence identity between GREP growth regulating proteins and the OsPSK growth regulating protein is extremely low, ranging from 9 to 18%. The GREP growth regulating proteins of the present invention are not retrievable from databases via BLAST searches using the OsPSK peptide sequence as query, in agreement with previous reports stating that the OsPSK protein does not have significant homology to proteins in public databases (Yang et al., 2000). Also, the RNA expression profile of OsGREP1 is clearly different from that of OsPSK: the OsGREP1 gene is highly expressed in intact plant tissues and much less in suspension culture cells whereas the OsPSK gene is highly expressed in tissue culture cells but not in intact plant tissues.

As used herein, nucleic acids are written left to right in 5′ to 3′ orientation, unless otherwise indicated; amino acid sequences are written left to right in amino to carboxy orientation, respectively. Numeric ranges are inclusive of the numbers defining the range. Amino acids may be referred to herein either by their commonly known three letter symbols or by the one-letter symbols recommended by the IUPAC-IUB Biochemical Nomenclature Commission. Nucleotides are referred to by their commonly accepted single-letter codes or by IUB codes for degenerate positions.

The terms ‘gene(s)’, ‘polynucleotide’, ‘nucleic acid’, ‘nucleotide sequence’, or ‘nucleic acid molecule(s)’ as used herein, refer to a polymeric form of a deoxyribonucleotide or ribonucleotide polymer of any length, either double- or single-stranded, or analogues thereof, that have the essential characteristic of a natural ribonucleotide in that they can hybridize to nucleic acids in a manner similar to naturally occurring polynucleotides; these terms are used interchangeable throughout the description. A great variety of modifications may be made to DNA and RNA that serve many useful purposes known to those skilled in the art. For example, methylation, ‘caps’ may be added and one or more of the naturally occurring nucleotides may be substituted with an analogue. Said terms also include peptide nucleic acids. The term polynucleotide as used herein includes such chemically, enzymatically or metabolically modified forms of polynucleotides. ‘Sense strand’ refers to a DNA strand that is homologous to a mRNA transcript thereof. ‘Antisense strand’ refers to the complementary strand of the sense strand.

By ‘encoding’ or ‘encodes’ with respect to a specified nucleotide sequence, is meant comprising the information for translation into a specified protein. A nucleic acid encoding a protein may contain non-translated sequences such as 5′ and 3′ untranslated regions (5′ and 3′ UTR) and introns or it may lack intron sequences such as for example in cDNAs. An ‘open reading frame’ or ‘ORF’ is defined as a nucleotide sequence that encodes a polypeptide.

The information by which a protein is encoded is specified by the use of codons. Typically, the amino acid sequence is encoded by the nucleic acid using the ‘universal’ genetic code but variants of this universal code exist (see for example Proc. Natl. Acad. Sci. U.S.A. 82: 2306-2309, 1985). The boundaries of the coding sequence are determined by a translation start codon at the 5′end and a translation stop codon at the 3′-terminus. As used herein ‘full-length sequence’ with respect to a specific nucleic acid or its encoded protein means having the entire amino acid sequence of a native protein. In the present invention, comparison to known full-length homologous (orthologous or paralogous) sequences is used to identify full-length sequences. Also, for a mRNA or cDNA, consensus sequences present at the 5′ and 3′ untranslated regions aid in the identification of a polynucleotide as full-length. For a protein, the presence of a start- and stopcodon aid in identifying the polypeptide as full-length. When the nucleic acid is to be expressed, advantage can be taken of known codon preferences or GC content preferences of the intended host as these preferences have been shown to differ (see e.g. http://www.kazusa.or.jp/codon/; Murray et al, 1989). Because of the degeneracy of the genetic code, a large number of nucleic acids can encode any given protein. As such, substantially divergent nucleic acid sequences can be designed to effect expression of essentially the same protein in different hosts. Conversely, genes and coding sequences essentially encoding the same protein isolated from different sources can consist of substantially different nucleic acid sequences.

The term ‘control sequence’ or ‘regulatory sequence’ refers to regulatory DNA sequences which are necessary to effect the expression of sequences to which they are ligated. The control sequences differ depending upon the intended host organism and upon the nature of the sequence to be expressed. For expression of a protein in prokaryotes, the control sequences generally include a promoter, a ribosomal binding site, and a terminator. In eukaryotes, control sequences generally include promoters, terminators and, in some instances, enhancers, and/or 5′ and 3′ untranslated sequences. The term ‘control sequence’ is intended to include, at a minimum, all components necessary for expression, and may also include additional advantageous components. As used herein, a ‘promoter’ includes reference to a region of DNA upstream from the transcription start and involved in binding RNA polymerase and other proteins to start transcription. Reference herein to a ‘promoter’ is to be taken in its broadest context and includes the transcriptional regulatory sequences derived from a classical eukaryotic genomic gene, including the TATA box which is required for accurate transcription initiation, with or without a CCMT box sequence and additional regulatory elements (i.e. upstream activating sequences, enhancers and silencers), which alter gene expression in response to developmental and/or external stimuli, or in a tissue-specific manner. The term ‘promoter’ also includes the transcriptional regulatory sequences of a classical prokaryotic gene, in which case it may include a −35 box sequence and/or a −10 box transcriptional regulatory sequences. The term ‘promoter’ is also used to describe a synthetic or fusion molecule, or derivative which confers, activates, or enhances expression of a nucleic acid molecule in a cell, tissue, or organ. A ‘plant promoter’ is a promoter capable of initiating transcription in plant cells. ‘Tissue-preferred promoters’ as used herein refers to promoters that preferentially initiate transcription in certain tissues such as for example in leaves, roots, etc. Promoters which initiate transcription only in certain tissues are referred herein as ‘tissue-specific’. Those skilled in the art will be aware that ‘inducible promoters’ have induced or increased transcription initiation in response to a developmental, chemical, environmental, or physical stimulus and that a ‘constitutive promoter’ is transcriptionally active during most, but not necessarily all phases of growth and development of a plant. Examples of constitutive plant promoters are given in Table 1. Examples of plant tissue-specific or tissue-preferred promoters are given in Table 2.

The term ‘terminator’ as used herein refers to a DNA sequence at the end of a transcriptional unit which signals 3′ processing and polyadenylation of a primary transcript and termination of transcription. Terminators comprise 3′-untranslated sequences with polyadenylation signals, which facilitate 3′ processing and the addition of polyadenylate sequences to the 3′-end of a primary transcript. Terminators active in cells derived from viruses, yeast, moulds, bacteria, insects, birds, mammals and plants are known and described in the literature. They may be isolated from bacteria, fungi, viruses, animals and/or plants.

TABLE 1
Exemplary constitutive plant promoters.

| GENE SOURCE | REFERENCE |
|---|---|
| Actin | McElroy et al., Plant Cell 2: 163-171, 1990. |
| CAMV 35S | Odell et al., Nature 313: 810-812, 1985. |
| CaMV 19S | Nilsson et al., Physiol. Plant. 100: 456-462, 1997. |
| GOS2 | de Pater et al., The Plant J. 2: 837-44, 1992. |
| Ubiquitin | Christensen et al., Plant Mol. Biol. 18: 675-689, 1992. |
| Rice cyclophilin | Buchholz et al., Plant Mol Biol. 25: 837-43, 1994. |
| Maize H3 histone | Lepetit et al., Mol. Gen. Genet. 231: 276-285, 1992. |
| Actin 2 | An et al., The Plant J. 10: 107-121, 1996. |

TABLE 2
Exemplary plant tissue-specific or tissue-preferred promoters

| | EXPRESSION | |
|---|---|---|
| GENE SOURCE | PATTERN | REFERENCE |
| α-amylase (Amy32b) | Aleurone | Lanahan, M B, et al., Plant Cell 4: 203-211, |
| | | 1992; Skriver, K, et al., Proc. Natl. |
| | | Acad. Sci. (USA) 88: 7286-7270, 1991. |
| Cathepsin β-like gene | Aleurone | Cejudo, F J, et al., Plant Mol. Biol. 20: |
| | | 849-856, 1992. |
| Agrobacterium rhizogenes rolB | Cambium | Nilsson et al., Physiol. Plant. 100: 456-462, |
| | | 1997. |
| PRP genes | cell wall | http://salus.medium.edu/mmg/tiemey/html |
| Chalcone synthase (chsA) | Flowers | Van der Meer et al., Plant Mol. Biol. 15: |
| | | 95-109, 1990. |
| LAT52 | Anther | Twell et al., Mol. Gen. Genet. 217: 240-245, |
| | | 1989. |
| Apetala-3 | Flowers | |
| Chitinase | fruit (berries, | Thomas et al., CSIRO Plant Industry, |
| | grapes, etc) | Urrbrae, South Australia, Australia; |
| | | http://winetitles.com.au/gwrdc/csh95-1.html |
| Rbcs-3A | Green tissue (eg | Lam et al., The Plant Cell 2: 857-866, |
| | leaf) | 1990; Tucker et al., Plant Physiol. 113: |
| | | 1303-1308, 1992. |
| Leaf-specific genes | Leaf | Baszczynski et al., Nucl. Acids Res. 16: |
| | | 4732, 1988. |
| Chlorella virus adenine | Leaf | Mitra and Higgins, Plant Mol. Biol. 26: 85-93, |
| methyltransferase gene | | 1994. |
| promoter | | |
| AldP gene promoter from rice | Leaf | Kagaya et al., Mol. and Gen. Genet. 248: |
| | | 668-674, 1995. |
| Rbcs promoter from rice or | Leaf | Kyozuka et al., Plant Physiol. 102: 991-1000, |
| tomato | | 1993. |
| Pinus cab-6 | Leaf | Yamamoto et al., Plant Cell Physiol. 35: |
| | | 773-778, 1994. |
| Rubisco promoter | Leaf | |
| Cab (chlorophyll a/b binding | Leaf | |
| protein) | | |
| SAM22 | Senescent leaf | Crowell et al., Plant Mol. Biol. 18: 459-466, |
| | | 1992. |
| Ltp gene (lipid transfer gene) | | Fleming et al., Plant J. 2: 855-862, 1992 |
| R. japonicum nif gene | Nodule | U.S. Pat. No. 4,803,165 |
| B. japonicum nifH gene | Nodule | U.S. Pat. No. 5,008,194 |
| GmENOD40 | Nodule | Yang et al., The Plant J. 3: 573-585, |
| | | 1993 |
| PEP carboxylase (PEPC) | Nodule | Pathirana et al., Plant Mol. Biol. 20: 437-450, |
| | | 1992. |
| Leghaemoglobin (Lb) | Nodule | Gordon et al., J. Exp. Bot. 44: 1453-1465, |
| | | 1993. |
| Tungro bacilliform virus gene | Phloem | Bhattacharyya-Pakrasi et al., The Plant J. |
| | | 4: 71-79, 1992. |
| Sucrose-binding protein gene | Plasma | Grimes et al., The Plant Cell 4: 1561-1574, |
| | membrane | 1992. |
| Pollen-specific genes | Pollen; | Albani et al., Plant Mol. Biol. 15: 605, |
| | microspore | 1990; Albani et al., Plant Mol. Biol. 16: |
| | | 501, 1991. |
| Zm13 | Pollen | Guerrero et al., Mol. Gen. Genet. 224: |
| | | 161-168, 1993. |
| Apg gene | Microspore | Twell et al., Sex. Plant Reprod. 6: 217-224, |
| | | 1993. |
| Maize pollen-specific gene | Pollen | Hamilton et al., Plant Mol. Biol. 18: 211-218, |
| | | 1992. |
| Sunflower pollen-expressed | Pollen | Baltz et al., The Plant J. 2: 713-721, |
| gene | | 1992. |
| B. napus pollen-specific gene | Pollen; anther | Arnoldo et al., J. Cell. Biochem., Abstract |
| | tapetum | No. Y101, 204, 1992. |
| Root-expressible genes | Roots | Tingey et al., EMBO J. 6: 1, 1987. |
| Tobacco auxin-inducible gene | Root tip | Van der Zaal et al., Plant Mol. Biol. 16, |
| | | 983, 1991. |
| β-tubulin | Root | Oppenheimer et al., Gene 63: 87, 1988. |
| Tobacco root-specific genes | Root | Conkling et al., Plant Physiol. 93: 1203, |
| | | 1990. |
| B. napus G1-3b gene | Root | U.S. Pat. No. 5,401,836 |
| SbPRP1 | Roots | Suzuki et al., Plant Mol. Biol. 21: 109-119, |
| | | 1993. |
| AtPRP1; AtPRP3 | Roots; root hairs | http://salus.medium.edu/mmg/tierney/html |
| RD2 gene | root cortex | http://www2.cnsu.edu/ncsu/research |
| TobRB7 gene | root vasculature | http://www2.cnsu.edu/ncsu/research |
| AtPRP4 | Leaves; flowers; | http://salus.medium.edu/mmg/tierney/html |
| | lateral root | |
| | primordia | |
| Seed-specific genes | Seed | Simon et al., Plant Mol. Biol. 5: 191, |
| | | 1985; Scofield et al., J. Biol. Chem. 262: |
| | | 12202, 1987; Baszczynski et al., Plant |
| | | Mol. Biol. 14: 633, 1990. |
| Brazil Nut albumin | Seed | Pearson et al., Plant Mol. Biol. 18: 235-245, |
| | | 1992. |
| Legumin | Seed | Ellis et al., Plant Mol. Biol. 10: 203-214, |
| | | 1988. |
| Glutelin (rice) | Seed | Takaiwa et al., Mol. Gen. Genet. 208: 15-22, |
| | | 1986; Takaiwa et al., FEBS Letts. |
| | | 221: 43-47, 1987. |
| Zein | Seed | Matzke et al., Plant Mol. Biol., 14: 323-332, |
| | | 1990. |
| NapA | Seed | Stalberg et al., Planta 199: 515-519, |
| | | 1996. |
| Wheat LMW and HMW | Endosperm | Colot et al., Mol Gen Genet 216: 81-90, |
| glutenin-1 | | 1989; NAR 17: 461-462, 1989 |
| Wheat SPA | Seed | Albani et al, Plant Cell, 9: 171-184, 1997. |
| Wheat α, β, γ-gliadins | Endosperm | Rafalski et al., EMBO 3: 1409-15, 1984 |
| Barley ltr1 promoter | Endosperm | |
| Barley B1, C, D-hordein | Endosperm | Cho et al., Theor Appl Gen 98: 1253-1262, |
| | | 1999; Mueller & Knudsen, The |
| | | Plant J. 4: 343-355, 1993; Mol Gen |
| | | Genet 250: 750-760, 1996. |
| Barley DOF | Endosperm | Mena et al., The Plant J. 116: 53-62, |
| | | 1998. |
| Blz2 | Endosperm | EP99106056.7 |
| Synthetic promoter | Endosperm | Vicente-Carbajosa et al., The Plant J. 13: |
| | | 629-640, 1998. |
| Rice prolamin NRP33 | Endosperm | Wu et al., Plant Cell Physiol. 39: 885-889, |
| | | 1998 |
| Rice α-globulin Glb-1 | Endosperm | Wu et al., Plant Cell Physiol. 39: 885-889, |
| | | 1998 |
| Rice OSH1 | Embryo | Sato et al., Proc. Natl. Acad. Sci. USA, |
| | | 93: 8117-8122, 1996. |
| Rice α-globulin REB/OHP-1 | Endosperm | Nakase et al., Plant Mol. Biol. 33: 513-522, |
| | | 1997. |
| Rice ADP-glucose PP | Endosperm | Trans. Res. 6: 157-168, 1997. |
| Maize ESR gene family | Endosperm | Opsahl-Ferstad et al., The Plant J. 12: |
| | | 235-246, 1997. |
| Sorghum γ-kafirin | Endosperm | DeRose R T et al., Plant Mol. Biol. 32: |
| | | 1029-1035, 1996. |
| KNOX | Embryo | Postma-Haarsma et al., Plant Mol. Biol. |
| | | 39: 257-271, 1999. |
| Rice oleosin | Embryo and | Wu et al., J. Biochem., 123: 386, 1998. |
| | aleuron | |
| Sunflower oleosin | seed (embryo and | Cummins et al., Plant Mol. Biol. 19: 873-876, |
| | dry seed) | 1992. |
| LEAFY | Shoot meristem | Weigel et al., Cell 69: 843-859, 1992. |
| Arabidopsis thaliana knat1 | Shoot meristem | Accession number AJ131822 |
| Malus domestica kn1 | Shoot meristem | Accession number Z71981 |
| CLAVATA1 | Shoot meristem | Accession number AF049870 |
| Stigma-specific genes | Stigma | Nasrallah et al., Proc. Natl. Acad. Sci. |
| | | USA 85: 5551, 1988; Trick et al., Plant |
| | | Mol. Biol. 15: 203, 1990. |
| Class I patatin gene | Tuber | Liu et al., Plant Mol. Biol. 153: 386-395, |
| | | 1991. |
| PCNA rice | Meristem | Kosugi et al., Nucl. Acids Res. 19: 1571-1576, |
| | | 1991; Kosugi S. and Ohashi Y, |
| | | Plant Cell 9: 1607-1619, 1997. |
| Pea TubA1 tubulin | Dividing cells | Stotz and Long, Plant Mol. Biol. 41: 601-614, |
| | | 1999. |
| Arabidopsis cdc2a | Cycling cells | Chung and Parish, FEBS Lett, 362: 215-219, |
| | | 1995. |
| Arabidopsis Rop1A | Anthers; mature | Li et al., Plant Physiol.118: 407-417, |
| | pollen + pollen | 1998. |
| | tubes | |
| Arabidopsis AtDMC1 | Meiosis- | Klimyuk and Jones, The Plant J. 11: 1-14, |
| | associated | 1997. |
| Pea PS-IAA4/5 and PS-IAA6 | Auxin-inducible | Wong et al., Plant J. 9: 587-599, 1996. |
| Pea farnesyltransferase | Meristematic | Zhou et al., Plant J. 12: 921-930, 1997. |
| | tissues; phloem | |
| | near growing | |
| | tissues; light- and | |
| | sugar-repressed | |
| Tobacco (N. sylvestris) cyclin | Dividing cells/ | Trehin et al., Plant Mol. Biol. 35: 667-672, |
| B1; 1 | meristematic | 1997. |
| | tissue | |
| Catharanthus roseus | Dividing cells/ | Ito et al., The Plant J. 11: 983-992, 1997. |
| Mitotic cyclins CYS (A-type) | meristematic | |
| and CYM (B-type) | tissue | |
| Arabidopsis cyc1At (=cyc B1; 1) | Dividing cells/ | Shaul et al., Proc. Natl. Acad. Sci. U.S.A |
| and cyc3aAt (A-type) | meristematic | 93: 4868-4872, 1996. |
| | tissue | |
| Arabidopsis tef1 promoter box | Dividing cells/ | Regad et al., Mol. Gen. Genet. 248: 703-711, |
| | meristematic | 1995. |
| | tissue | |
| Catharanthus roseus cyc07 | Dividing cells/ | Ito et al., Plant Mol. Biol. 24: 863-878, |
| | meristematic | 1994. |
| | tissue | |

The term ‘operably linked’ as used herein refers to a juxtaposition wherein the components so described are in a relationship permitting them to function in their intended manner. A control sequence ‘operably linked’ to a coding sequence is ligated in such a way that expression of the coding sequence is achieved under conditions compatible with the control sequences. In case the control sequence is a promoter, a double-stranded nucleic acid is used.

The term ‘hybridizing’ includes reference to formation of a duplex nucleic acid structure through annealing of two (partially or completely) complementary single-stranded nucleic acid sequences. The hybridization process can occur entirely in solution, e.g. the polymerase chain reaction process, subtractive hybridization, and cDNA synthesis. Alternatively, one of the complementary nucleic acids may be immobilized on a solid support such as on a nylon membrane in DNA and RNA gel blot analyses, or on a siliceous glass support for microarray hybridization. Other uses and techniques relying on hybridization are well known to those skilled in the art. The critical factors for hybridization are the ionic strength and temperature of the solution and characteristics of the nucleic acids such as length and % GC content. The Tₘ is the temperature at which 50% of a complementary target sequence hybridizes to a perfectly matched probe under defined ionic strength and pH. For DNA-DNA hybrids, the Tₘ can be calculated from the equation of Meinkoth and Wahl (1984): Tₘ=81.5 C+16.6 (logM)+0.41 (% GC)−0.61 (% formamide)−500/L where M is the molarity of monovalent cations, % GC is the percentage of guanosine and cytosine nucleotides in the DNA, % formamide is the percentage of formamide in the hybridization solution, and L is the length of the hybrid in base pairs. The terms ‘stringent conditions’ or ‘stringent hybridization conditions’ includes reference to conditions under which a probe will hybridize to its target sequence to a detectable greater degree than other sequences (e.g., at least 2-fold over background). Stringent conditions are sequence-dependent and will be different in different circumstances. By controlling the stringency of the hybridization and/or washing conditions, target sequences can be identified which are 100% complementary to the probe. Alternatively, stringency conditions can be adjusted to allow some mismatching so that sequences with lower degrees of identity are detected. Stringent conditions are those in which the salt concentration is less than about 1.5M Na ion, typically 0.01 to 1.0 M Na ion (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. An example of low stringency conditions includes hybridization with a buffer solution of 30 to 35% formamide, 1M NaCl, 1% SDS (sodium dodecyl sulphate) at 37° C., and a wash in 1× to 2×SSC (20×SSC is 3.0 M NaCl/0.3M trisodium citrate) at 50° to 55° C. Exemplary moderate stringency conditions include hybridization in 40 to 45% formamide, 1M NaCl, 1% SDS at 37° C., and a wash in 0.5×SSC to 1.0×SSC at 55′ to 60° C. Exemplary high stringency conditions include hybridization in 50% formamide, 1M NaCl, 1% SDS at 37° C., and a wash in 0.1×SSC at 60° to 65° C. Specificity is typically the function of post-hybridization washes. Those skilled in the art will understand that the conditions for hybridization and washing can be adjusted to achieve hybridization to sequences of the desired identity. A guide to the hybridization of nucleic acids is found in Sambrook, Molecular Cloning; A Laboratory Manual, 2nd Edition, Cold Spring Harbor Laboratory Press, Cold Spring Harbor, N.Y. (1989); and in Tijssen, Laboratory Techniques in Biochemistry and Molecular Biology-Hybridization with Nucleic Acid Probes, Part I, Chapter 2 ‘Overview of principles of hybridization and the strategy of nucleic acid probe assays’, Elsevier, N.Y. (1993), which disclosures are incorporated by reference as if fully set forth.

The terms ‘protein’ and ‘polypeptide’ are interchangeably used in this application and refer to a polymer of amino acids. These terms do not refer to a specific length of the molecule and thus peptides and oligopeptides are included within the definition of polypeptide. This term also refers to or includes post-translational modifications of the polypeptide, for example, glycosylations, acetylations, phosphorylations, sulfations and the like. These modifications are well known to those skilled in the art and examples are described by Wold F., Posttranslational Protein Modifications: Perspectives and Prospects, pp. 1-12 in Posttranslational Covalent Modification of Proteins, B. C. Johnson, Ed., Academic Press, New York (1983) and Seifter et al. (1990). Included within the definition are, for example, polypeptides containing one or more analogues of an amino acid (including, for example, unnatural amino acids, etc.), polypeptides with substituted linkages, as well as other modifications known in the art, that are both naturally occurring and non-naturally.

The term ‘amino acid’, ‘amino acid residue’ or ‘residue’ are used interchangeably herein to refer to an amino acid that is incorporated into a protein, polypeptide, or peptide. The amino acid may be a naturally occurring amino acid and may be a known analogue of natural amino acids that can function in a similar manner as naturally occurring amino acids.

TABLE 3
Properties of naturally occurring amino acids.

| Charge | | |
|---|---|---|
| Properties/hydrophobicity | Side Group | Amino Acid |
| Nonpolar hydrophobic | aliphatic | ala, ile, leu, val |
| | aliphatic, S-containing | met |
| | aromatic | phe, trp |
| | imino | pro |
| Polar uncharged | aliphatic | gly |
| | amide | asn, gln |
| | aromatic | tyr |
| | hydroxyl | ser, thr |
| | sulfhydryl | cys |
| Positively charged | basic | arg, his, lys |
| Negatively charged | acidic | asp, glu |

As used herein ‘homologues’ of a protein of the invention are those peptides, oligopeptides, polypeptides, proteins and enzymes which contain amino acid substitutions, deletions and/or additions relative to said protein, providing similar biological activity as the unmodified polypeptide from which they are derived. To produce such homologues, amino acids present in the protein can be replaced by other amino acids having similar properties, for example hydrophobicity, hydrophilicity, antigenicity, propensity to form or break α-helical structures or β-sheet structures, and so on. Conservative substitution tables are well known in the art (see for example Creighton (1984) Proteins. W.H. Freeman and Company) and are used in sequence alignment software packages. An overview of physical and chemical properties of amino acids is given in Table 3.

Substitutional variants of a protein of the invention are those in which at least one residue in the amino acid sequence has been removed and a different residue inserted in its place.

Amino acid substitutions are typically of single residues, but may be clustered depending upon functional constraints placed upon the polypeptide; insertions will usually be of the order of about 1-10 amino acid residues, and deletions will range from about 1-20 residues. Preferably, amino acid substitutions will comprise conservative amino acid substitutions, such as those described supra. Insertional amino acid sequence variants of a protein of the invention are those in which one or more amino acid residues are introduced into a predetermined site in said protein. Insertions can comprise amino-terminal and/or carboxy-terminal fusions as well as intra-sequence insertions of single or multiple amino acids. Generally, insertions within the amino acid sequence will be smaller than amino- or carboxy-terminal fusions, of the order of about 1 to 10 residues. Examples of amino- or carboxy-terminal fusion proteins or peptides include the binding domain or activation domain of a transcriptional activator as used in the yeast two-hybrid system, phage coat proteins, (histidine)₆-tag, glutathione S-transferase-tag, protein A, maltose-binding protein, dihydrofolate reductase, Tag●100 epitope (EETARFQPQPGYRS), c-myc epitope (EQKLISEEDL), FLAG®-epitope (DYKDDDK), lacZ, CMP (calmodulin-binding peptide), HA epitope (YPYDVPDYA), protein C epitope (EDQVDPRLIDGK) and VSV epitope (YTDIEMNRLGK). Deletion variants of a protein of the invention are characterized by the removal of one or more amino acids from said protein. Amino acid variants of a protein of the invention may readily be made using peptide synthetic techniques well known in the art, such as solid phase peptide synthesis and the like, or by recombinant DNA manipulations. The manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols.

‘Derivatives’ of a protein of the invention are those peptides, oligopeptides, polypeptides, proteins and enzymes which comprise at least about five contiguous amino acid residues of said polypeptide but which retain the biological activity of said protein. A ‘derivative’ may further comprise additional naturally-occurring, altered glycosylated, acylated or non-naturally occurring amino acid residues compared to the amino acid sequence of a naturally-occurring form of said polypeptide. A derivative may also comprise one or more non-amino acid substitutents compared to the amino acid sequence of which it is derived, for example a reporter molecule or other ligand, covalently or non-covalently bound to the amino acid sequence such as, for example, a reporter molecule which is bound to facilitate its detection. The term ‘antibody’ as used herein typically refers to a polypeptide substantially encoded by an immunoglobulin gene or immunoglobulin genes or fragments thereof, which specifically bind and recognize a substance termed the antigen. Those skilled in the art will appreciate that such fragments may be derived from an intact antibody by proteolytic digestion or may be synthesized de novo either chemically or by recombinant DNA methodology. Therefore, the term antibody, as used herein, also includes antibody fragments such as single chain Fv, chimeric antibodies (i.e., comprising constant and variable regions from different species), humanized antibodies (i.e., comprising a complementarity determining region from a non-human source), heteroconjugate antibodies (e.g. bispecific antibodies) and plantibodies. The term antibody furthermore includes derivatives thereof such as labelled antibodies. Examples of antibody labels include alkaline phosphatase, peroxidase, and radiolabels. Other labels are known to persons skilled in the art. Many molecular biology techniques rely on the use of antibodies including protein gel blot analysis, protein quantitation methods such as ELISA, immunoaffinity purification of proteins, and immunoprecipitation, to name just a few. Other uses of antibodies and of peptide antibodies are known to those skilled in the art.

The term ‘antigen’ as used herein refers to a substance to which an antibody can be generated and/or to which the antibody is specifically immunoreactive. The specific immunoreactive sites within the antibody are termed epitopes or antigenic determinants. Immunogens are substances capable of eliciting an immune response. Those skilled in the art will recognize that all immunogens are antigens but some antigens, such as haptens, are not immunogens but can be made immunogenic by binding to a carrier molecule. An antibody immunologically reactive with a particular antigen can be generated in vivo or by recombinant methods such as selection of libraries of recombinant antibodies in phage or similar vectors. See, e.g., Huse et al., 1989; Ward et al., 1989; and Vaughan et al., 1996).

The term ‘immunologically active’ is meant to include a molecule or specific fragments thereof, such as epitopes or haptens which are recognized by, i.e., bind to antibodies.

As used herein, the term ‘heterologous’ in reference to a nucleic acid is a nucleic acid that is either derived from a cell or organism with a different genomic background, or, if from the same genomic background, is substantially modified from its native form in composition and/or genomic environment through deliberate human manipulation. Similarly, a heterologous protein may originate from a different species, or, if from the same species, it may be substantially modified by human manipulation. The vector or nucleic acid molecule according to the invention may either be integrated into the genome of the host cell or it may be maintained in some form extrachromosomally. In this respect, it is also to be understood that the nucleic acid molecule of the invention can be used to restore or create a mutant gene via homologous recombination or via other molecular mechanisms such as for example RNA interference (Paszkowski, 1994).

The term ‘recombinant DNA molecule’ or ‘chimeric gene’ includes a hybrid DNA produced by joining pieces of DNA from different sources through deliberate human manipulation.

The term ‘expression’ means the production of a protein or nucleotide sequence in the cell or cell-free system. It includes transcription into an RNA product, and/or translation to a protein product or polypeptide from a DNA encoding that product, as well as possible post-translational modifications. Depending on the specific constructs and conditions used, the protein may be recovered from the cells, from the culture medium or from both. For the person skilled in the art it is well known that it is not only possible to express a native protein but also to express the protein as fusion polypeptides or to add signal sequences directing the protein to specific compartments of the host cell, e.g., ensuring transport of the peptide to a chloroplast, ensuring secretion of the peptide into the culture medium, etc. Furthermore, such a protein and fragments thereof can be chemically synthesized and/or modified according to standard methods described.

A ‘vector’ as used herein, includes reference to a nucleic acid used for transfection or transformation of a host cell and into which a nucleic acid can be inserted. Expression vectors allow transcription and/or translation of a nucleic acid inserted therein. Expression vectors can, for instance, be cloning vectors, binary vectors or integrating vectors. Vectors may contain regulatory sequences to ensure expression in prokaryotic and/or eukaryotic cells. In the case of eukaryotic cells, vectors normally comprise (i) promoters ensuring initiation of transcription, and (ii) terminators, which contain polyadenylation signals ensuring 3′ processing, polyadenylation of a primary transcript, and termination of transcription. For example, the promoter of the ³⁵S RNA from Cauliflower Mosaic Virus (CaMV) is frequently used in plant transformation studies. Other promoters commonly used in plants are the polyubiquitin promoter and the actin promoter for ubiquitous expression. The termination signals usually employed are from the nopaline synthase gene or the CaMV ³⁵S terminator. Additional regulatory elements may include transcriptional as well as translational enhancers. A plant translational enhancer often used is the Tobacco Mosaic Virus omega sequence. The inclusion of an intron has been shown to increase expression levels by up to 100-fold in certain plants (Mait, 1997; Ni, 1995). Regulatory elements permitting expression in prokaryotic host cells comprise, e.g., the P_{L}, lac, tip or tac promoter in E. coli. Examples of regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. In this context, suitable expression vectors are known in the art such as Okayama-Berg cDNA expression vector pcDV1 (Pharmacia), pCDM8, pRc/CMV, pcDNA1, pcDNA3 (Invitrogen), pSPORT1 (GIBCO BRL).

Advantageously, vectors of the invention comprise a selectable and/or scorable marker. Selectable marker genes useful for the selection of transformed plant cells, callus, plant tissue and plants are well known to those skilled in the art. For example, antimetabolite resistance provides the basis of selection for: the dhfr gene, which confers resistance to methotrexate (Reiss, 1994); the npt gene, which confers resistance to the aminoglycosides neomycin, kanamycin and paromomycin (Herrera-Estrella, 1983); and hpt, which confers resistance to hygromycin (Marsh, 1984). Additional selectable markers genes have been described, namely trpB, which allows cells to utilize indole in place of tryptophan; hisD, which allows cells to utilize histinol in place of histidine (Hartman, 1988); mannose-6-phosphate isomerase which allows cells to utilize mannose (WO 94/20627) and omithine decarboxylase which confers resistance to the ornithine decarboxylase inhibitor, 2-(difluoromethyl)-DL-omithine or DFMO (McConlogue, 1987) or deaminase from Aspergillus terreus which confers resistance to Blasticidin S (Tamura, 1995). Useful scorable markers are also known to those skilled in the art and are commercially available. For example, the genes encoding luciferase (Giacomin, 1996; Scikantha, 1996), green fluorescent protein (Gerdes, 1996) or β-glucuronidase (Jefferson, 1987) may be used.

As used herein, a ‘host cell’ is a cell that contains a vector and supports the expression and/or replication of this vector. Host cells may be prokaryotic cells such as E. coli and A. tumefaciens, or may be eukaryotic cells such as yeast, insect, amphibian, plant or mammalian cells. Preferably, host cells are monocotyledonous or dicotyledonous plant cells. The terms “fragment of a sequence”, “part of a sequence”, or “part thereof” mean a truncated sequence of the original sequence referred to. The truncated sequence (nucleic acid or protein sequence) can vary widely in length; the minimum size being a sequence of sufficient size to provide a sequence with at least a comparable function and/or activity of the original sequence referred to, while the maximum size is not critical. In some applications, the maximum size usually is not substantially greater than that required to provide the desired activity and/or function(s) of the original sequence. Typically, the truncated amino acid sequence will range from about 5 to about 60 amino acids in length. More typically, however, the sequence will be a maximum of about 50 amino acids in length, preferably a maximum of about 30 amino acids. It is usually desirable to select sequences of at least about 10, 12 or 15 amino acids, up to a maximum of about 20 or 25 amino acids.

Methods for alignment of nucleic acid and protein sequences for comparative studies are well known in the art. Several algorithms have been described for optimal global sequence alignment, i.e. the alignment of two nucleic acid or protein sequences over their entire length, including that one of Smith and Waterman (1981); Needleman and Wunsch (1970) and Pearson and Upman (1988). Examples of computerized implementations of such algorithms are: CLUSTAL, described by Higgins and Sharp (1988); Pearson et al. (1994); and GAP, PILEUP and others included in the Wisconsin Genetics Software Package, Genetics Qomputer Group (GCG), 575 Science Dr., Madison, Wis., U.S.A. PileUp creates a multiple sequence alignment using a simplification of the progressive alignment method of Feng and Doolittle (1987). The method used is similar to the method described by Higgins and Sharp (1989). PileUp can also plot a tree showing the clustering relationships used to create the alignment. As used herein, ‘sequence identity’ in the context of two polypeptide sequences includes reference to the residues in the two sequences which are in the same position when aligned for maximum correspondence. With respect to polypeptide sequence alignment, those skilled in the art will recognize that aligned residues which are not identical may be conservative amino acid substitutions where amino acid residues are substituted for other amino acid residues with similar physicochemical properties (see supra Table 3).

Sequences which differ by such conservative substitutions are said to have sequence similarity and the percent identity may be adjusted upwards to correct for the conservative nature of the substitution. As used herein percentage of sequence identity means the percentage calculated by determining the number of positions at which an identical amino acid residue occurs in both sequences (i.e. the number of matched positions), divided by the total number of positions and multiplied by 100. For purposes of the present invention, alignments were performed using ClustalX version 1.81 with minor manual alignment modification. The % identity and similarity report is calculated with the Genedoc program 2.1 based on the alignment.

As used herein, ‘query’ is a defined sequence that is used as a basis for alignment using the BLAST (Basic Local Alignment Search Tool) family of programs (see http://www.ncbi.nlm.nih.gov/BLAST/). A query may be a subset or the entirety of a specified sequence; for example it may be a full-length cDNA or a part thereof, a complete ORF or a part thereof. The BLAST software package includes: blastn to compare a nucleotide query sequence against a nucleotide sequence database; blastp to compare an amino acid query sequence against a protein sequence database; blastx to compare a nucleotide query sequence translated in all reading frames against a protein sequence database; tblastn to compare a protein query sequence against a nucleotide sequence database dynamically translated in all reading frames; tblastx to compare the six-frame translations of a nucleotide query sequence against the six-frame translations of a nucleotide sequence database. Instead of identifying optimal global alignments, BLAST aims to identify regions of optimal local alignment, i.e. the alignment of some portion of two nucleic acid or protein sequences, to detect relationships among sequences which share only isolated regions of similarity (Altschul et al., 1990). The E-value is used to indicate the expectation value. The lower the E value, the more significant the alignment. See the National Center for Biotechnology Information (NCBI) website for a complete description on E-value (http://www.ncbi.nlm.nih.gov/BLAST/tutorial/). In the present invention, the BLAST 2.0 suite of programs using default parameters was used (Altschul et al., 1997). Blast searches were performed on a local server or remotely through the NCBI server against publicly available databases present locally or at the NCBI website (http://www.ncbi.nim.nih.gov/) or at The Institute for Genomics Research (TIGR) website (http://www.tigr.org/tdb/).

As used herein, the term ‘plant’ includes reference to whole plants, plant organs (such as leaves, roots, stems, etc.), seeds and plant cells and progeny of same. ‘Plant cell’, as used herein, includes suspension cultures, embryos, meristematic regions, callus tissue, leaves, seeds, roots, shoots, gametophytes, sporophytes, pollen, and microspores. The plants that can be used in the methods of the invention include all plants which belong to the superfamily Viridiplantae, including both monocotyledonous and dicotyledonous plants. A particularly preferred plant is rice (Oryza sativa L.).

The term “transformation” as used herein, refers to the transfer of an exogenous polynucleotide into a host cell, irrespective of the method used for the transfer. The polynucleotide may be transiently or stably introduced into the host cell and may be maintained non-integrated, for example, as a plasmid, or alternatively, may be integrated into the host genome. Methods for the introduction of foreign DNA into plants are also well known in the art These include, for example, the transformation of plant cells or tissues with T-DNA using Agrobacterium tumefaciens or Agrobacterium rhizogenes, the fusion of protoplasts, direct gene transfer (see, e.g., EP-A 164 575), injection, electroporation, biolistic methods like particle bombardment, pollen-mediated transformation, plant virus-mediated transformation, liposome-mediated transformation, transformation using wounded or enzyme-degraded immature embryos, or wounded or enzyme-degraded embryogenic callus and other methods known in the art. The vectors used in the method of the invention may contain further functional elements, for example “left border”- and “right border”-sequences of the T-DNA of Agrobacterium which allow for stable integration into the plant genome. Furthermore, methods and vectors are known to the person skilled in the art which permit the generation of marker free transgenic plants, i.e. the selectable or scorable marker gene is lost at a certain stage of plant development or plant breeding. This can be achieved e.g., by, cotransformation (Lyznik, 1989; Peng, 1995) and/or by using systems which utilize enzymes capable of promoting homologous recombination in plants (see, e.g., WO97/08331; Bayley, 1992; Lloyd, 1994; Maeser, 1991; Onouchi, 1991). Methods for the preparation of appropriate vectors are described by, e.g., Sambrook (Molecular Cloning; A Laboratory Manual, 2nd Edition (1989), Cold Spring Harbor Laboratory Press, Cold Spring Harbor, N.Y.). Suitable strains of Agrobacterium tumefaciens and vectors as well as transformation of Agrobacteria and appropriate growth and selection media are well known to those skilled in the art and are described in the prior art (GV3101 (pMK90RK), Koncz, 1986; C58C1 (pGV3850kan), Deblaere, 1985; Bevan, 1984; Koncz, 1989; Koncz, 1992; Koncz, (1994); EP-A-120 516; Hoekema, (1985), Chapter V, Fraley, 1986; An et al., 1985). Although the use of Agrobacterium tumefaciens is preferred in the method of the invention, other Agrobacterium strains, such as Agrobacterium rhizogenes, may be used, for example if a phenotype conferred by said strain is desired.

Methods for plant transformation using biolistic methods are well known to the person skilled in the art; see, e.g., Wan, 1994; Vasil, 1993 and Christou, 1996. Microinjection can be performed as described in Potrykus and Spangenberg (eds.), Gene Transfer To Plants. Springer Verlag, Berlin, N.Y. (1995). The transformation of most dicotyledonous plants is possible with the methods described above. The transformation of monocotyledonous plants may also be achieved using well-known methods such as biolistic methods as, e.g., described above, as well as protoplast transformation, electroporation of partially permeabilized cells, introduction of DNA using glass fibers, etc. Methods for transformation of monocotyledonous plants are well know in the art and include Agrobacterium-mediated transformation (Cheng et al., 1997—WO9748814; Hiei et al., 1994—WO9400977; Hiei et al., 1998—WO8717813; Rikiishi et al., 1999—WO9904618; Saito et al., 1995—WO9506722) and microprojectile bombardment (Adams et al., 1999—U.S. Pat. No. 5,969,213; Bowen et al., 1998—U.S. Pat. No. 5,736,369; Chang et al., 1994—WO9413822; Lundquist et al., 1999—U.S. Pat. No. 5,990,390; Walker et al, 1999—U.S. Pat. No. 5,955,362). Means for introducing recombinant DNA into plant tissue or cells include, but are not limited to, transformation using CaCl₂ and variations thereof, in particular the method described by Hanahan (J. Mol. Biol. 166: 557-560, 1983), direct DNA uptake into protoplasts (Krens et al., 1982; Paszkowski et al., 1984), PEG-mediated uptake to protoplasts (Armstrong et al., 1990) microparticle bombardment, electroporation (Fromm et al., 1985), microinjection of DNA (Crossway et al., 1986), microparticle bombardment of tissue explants or cells (Christou et al., 1988; Sanford, 1987), vacuum-infiltration of tissue with nucleic acid, or in the case of plants, T-DNA-mediated transfer from Agrobacterium to the plant tissue as described essentially by An et al. (1985), Herrera-Estrella et al. (1983a; 1983b; 1985), or in planta method using Agrobacterium tumefaciens such as that described by Bechtold et al. (1993) or Clough et al (1998), amongst others.

As used herein, ‘transgenic plant’ includes reference to a plant, which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a vector. ‘Transgenic’ is used herein to include any cell, cell line, callus, tissue, plant part or plant, the genotype of which has been altered by the presence of the heterologous nucleic acid including those transgenics initially so altered as well as those created by sexual crosses or asexual propagation from the initial transgenic.

Several documents are cited throughout the text of this specification. Each of the documents cited herein (including manufacturer's specifications, instructions, etc.) is hereby incorporated by reference as if fully set forth.

In accordance with the present invention, it has been discovered that a gene from rice (Oryza sativa), designated OsGREP1 for Oryza sativa Growth Regulating Protein 1, is involved in the submergence-induced growth of adventitious roots. It has also been discovered that the gene product of OsGREP1 belongs to a family of conserved proteins in rice and that homologous gene families occur ubiquitously in monocotyledonous and dicotyledonous plants. In addition, a new peptide consensus sequence termed the GREP signature motif has been discovered that is present in all members of these gene families.

The present invention provides an OsGREP1 gene and corresponding OsGREP1 protein from rice. The present invention also provides homologues of OsGREP1 from rice and other plants. As used herein, the terms ‘Growth Regulating Protein(s)’ or ‘GREP’ or ‘GREPs’ or ‘GREP protein(s)’ or ‘GREP growth regulating proteins’ refer to the gene products encoded by OsGREP1 or its homologues, analogues or paralogues.

In accordance with the present invention, it has been discovered that GREP growth regulating proteins from different plant species may have low overall amino acid sequence identity. It has also been discovered that GREP growth regulating proteins share several identifying characteristics as follows. GREP growth regulating proteins are small proteins with a molecular weight typically between 7.0 and 13 kD. GREP growth regulating proteins contain the consensus sequence:

CX₁X₂X₃CX₄X₅X₆X₇HX₈DYIYTX₉ (SEQ ID NO 52)

wherein X₁ are 4 to 8 amino acids, X₂ is D or E, X₃ is one or two amino acids, X₄ are two or three amino acids, X₅ is R or K, X₆ is R or K, X₇ are 4 to 5 amino acids, X₈ is any amino acid and X₉ is Q or H, or contain an amino acid sequence which is at least 90% identical, preferably at least 90.5%, 91%, 91.5%, 92%, 92.5%, 93%, 93.5%, 94%, 94.5%, 95%, identical, more preferably at least 95.5%, 96%, 96.5%, 97%, 97.5%, 98%, 98.5% identical, most preferably 99% or 99.5% identical identical to the sequence as represented in SEQ ID NO 52, and which sequence is also designated herein as “the GREP signature motif”. The GREP signature motif is located at the carboxy-terminus, and is preceded by an acidic domain and followed by a basic domain. GREP growth regulating proteins contain a hydrophobic peptide structure at their amino-terminus that may function as a signal peptide for targeting to the secretory pathway. GREP growth regulating proteins also have three α-helix structures in the post leader sequence.

Thus, the term GREP growth regulating proteins refers to proteins that contain the GREP signature motif. GREP growth regulating proteins have additional structural characteristics summarized above and described in detail in Example 6. Use herein of the term GREP or GREPs encompasses all such homologous or heterologous derivatives, homologues, and functional analogues. The GREP nucleotide sequence and corresponding protein may be native to a particular cell, i.e., is naturally occurring in such a cell, or may be heterologous to the cell, i.e., the genetic sequence or protein may be introduced into the cell from a source not originating from the same organism or may originate from the same organism or cell but present in a different genomic context. Thus, the present invention provides species-specific GREP genes that stimulate root growth and growth of specific plant tissues or organs in general. Transgenic plants may be produced by introduction of one or more GREP genes into their genome. The transgene may be placed under control of a defined regulatory sequence in order to produce the corresponding proteins and therefore enable the person skilled in the art to modify plant cell growth and/or development. The present invention also provides novel plant growth hormones that correspond to the gene products of the subject GREP genes and which may be used to contact plant material to modify the growth characteristics of such plant material.

The present invention also provides nucleic acid molecules comprising nucleotide sequences which code for a GREP growth regulating protein or a part thereof. For example, a nucleotide sequence which encodes the GREP consensus sequence (GREP signature motif) is provided as:

TGYN₁GAN₂TGYN₃MRNMRN₄CAYNNNGAYTAYATHTAYACNCAN (SEQ ID NO 53)

wherein M is A or C, R is A or G, Y is C or T, H is A or C or T, and N is G or A or T or C, and wherein N₁ is a stretch of 12 to 24 amino acid residues, N₂ is a stretch of 4 to 7 amino acid residues, N₃ is a stretch of 6 to 9 amino acid residues and N₄ is a stretch of 13 to 16 amino acid residues.

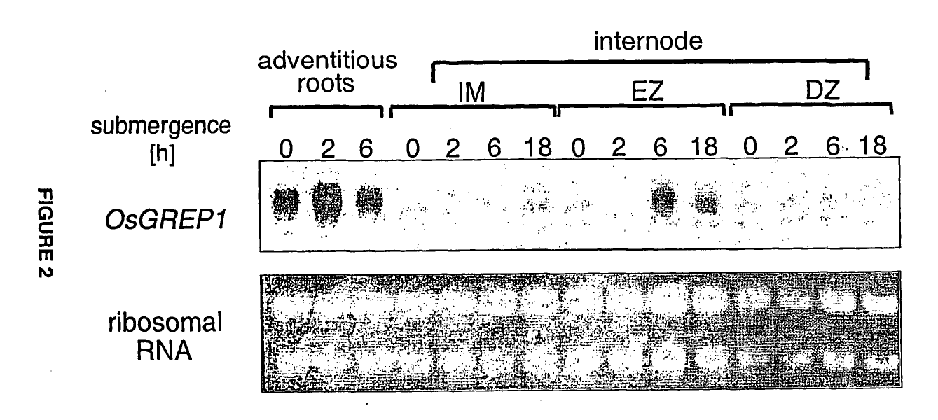

The nucleotide sequence of OsGREP1 is shown in FIG. 1a and is listed in the present specifications as SEQ ID NO 1. OsGREP1 bears an ORF of 119 amino acids (SEQ ID NO 2), encoding a protein with a calculated molecular weight of 12.7 kD. OsGREP1 shows high transcript levels in adventitious roots and is transiently induced upon submergence (see FIG. 2). Southern blot analysis under stringent conditions indicates that there are no sequences present in the genome of deepwater rice that are highly related (i.e. over 90% nucleotide sequence identity) to OsGREP1 (Example 3).

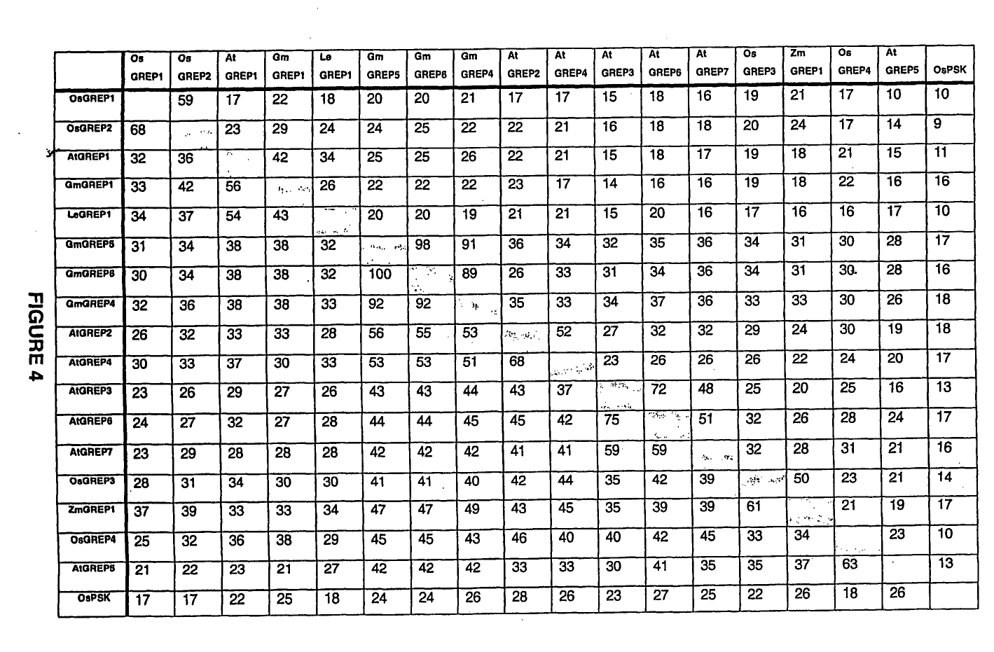

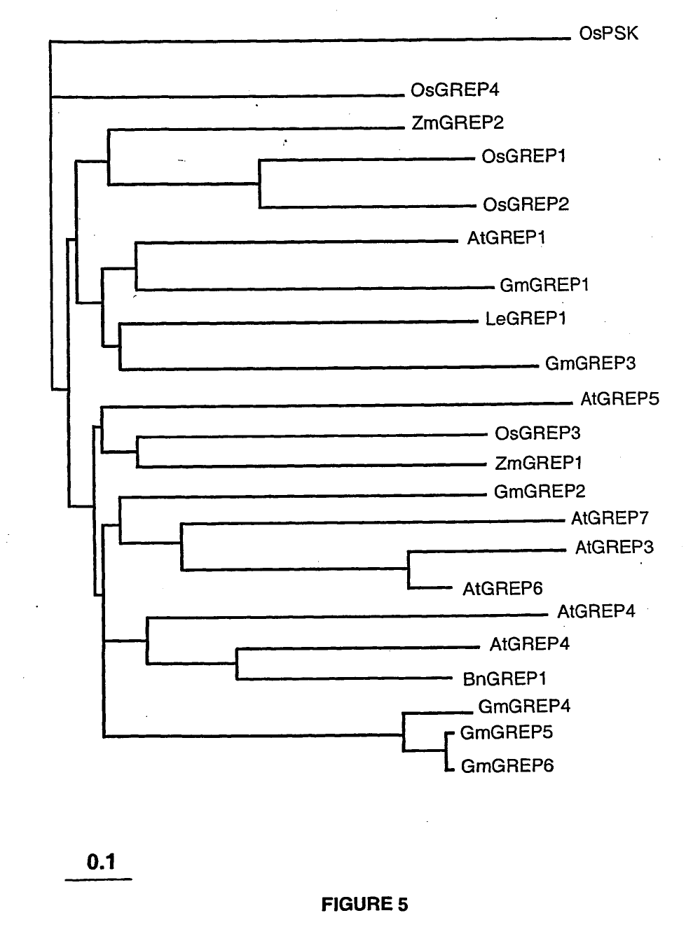

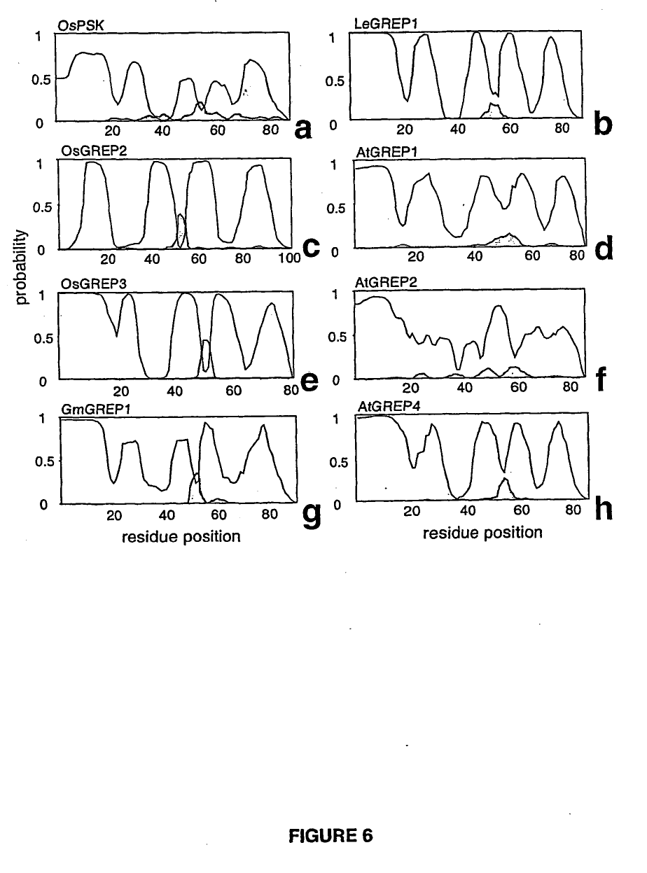

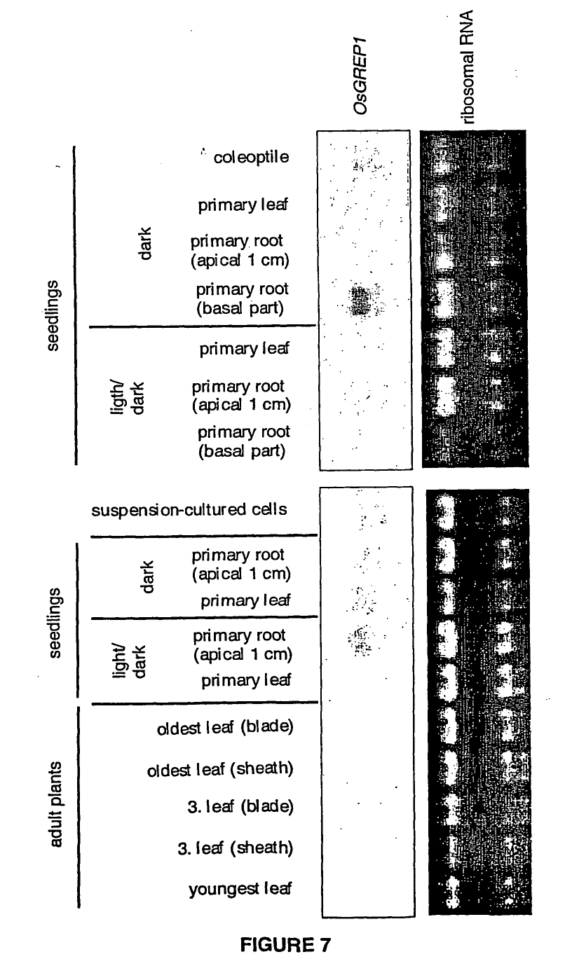

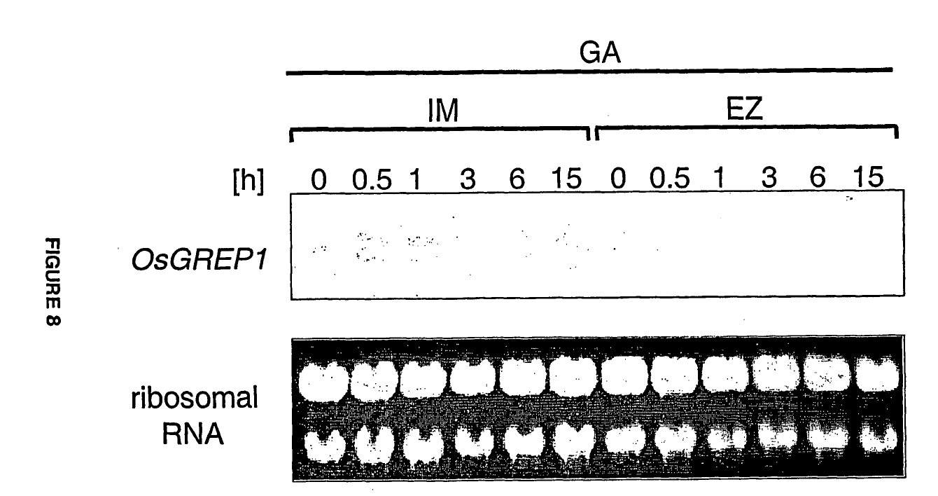

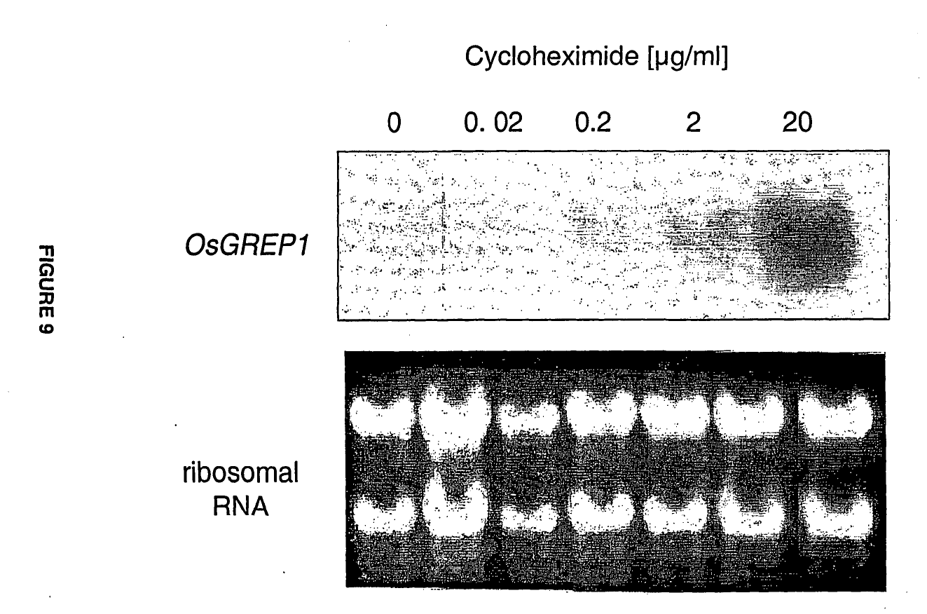

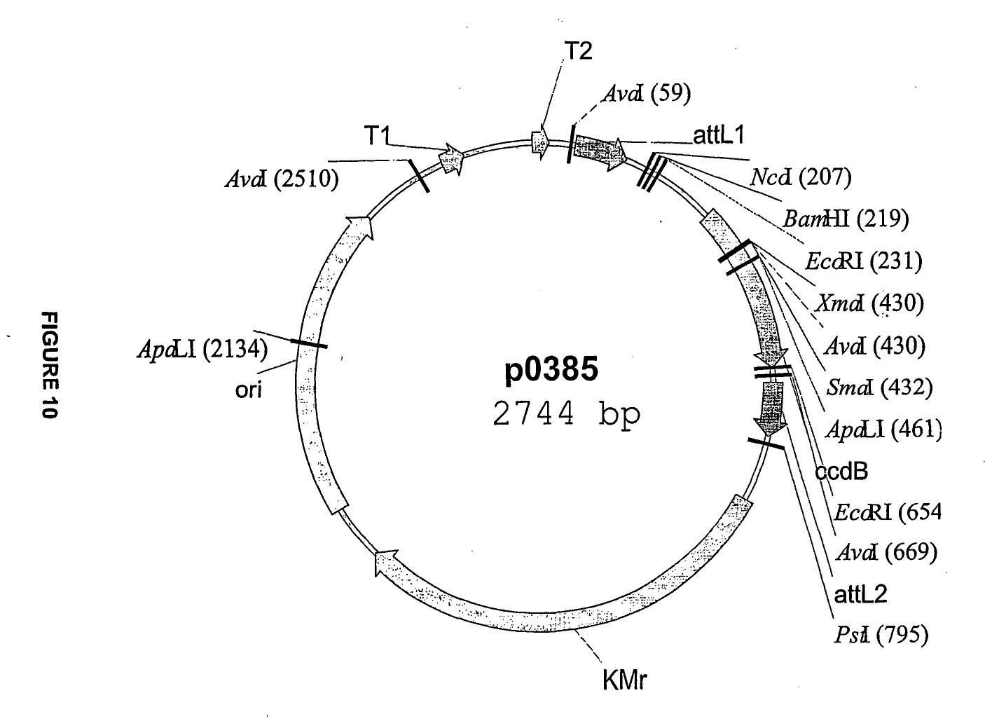

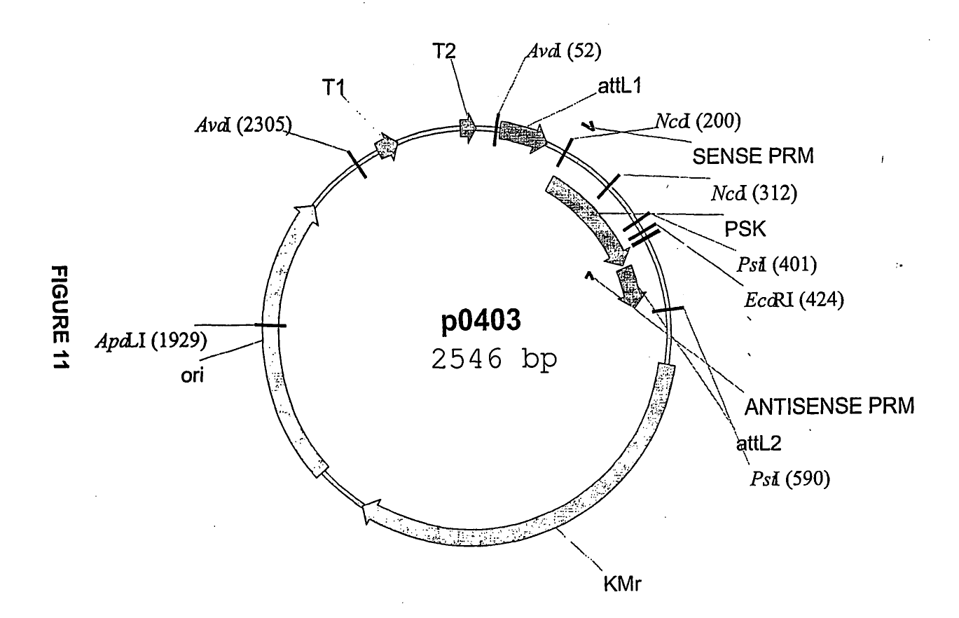

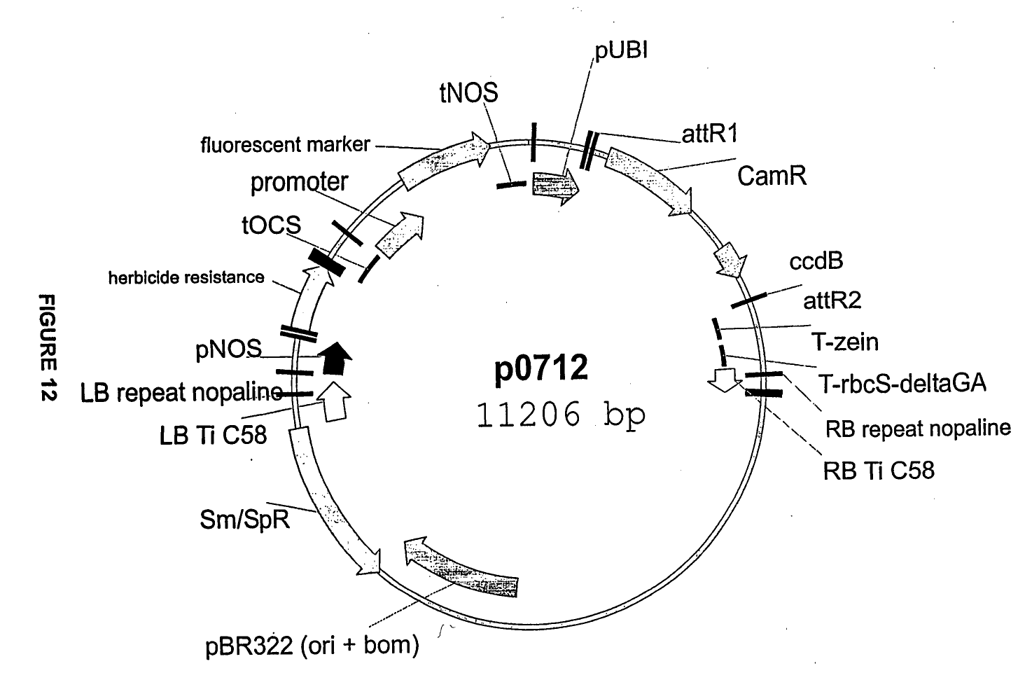

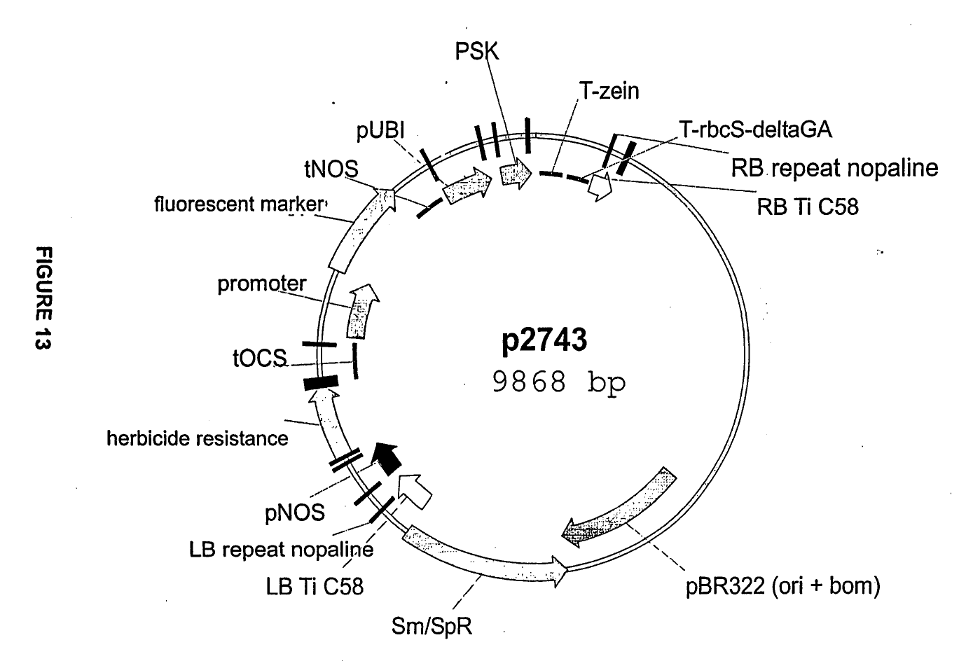

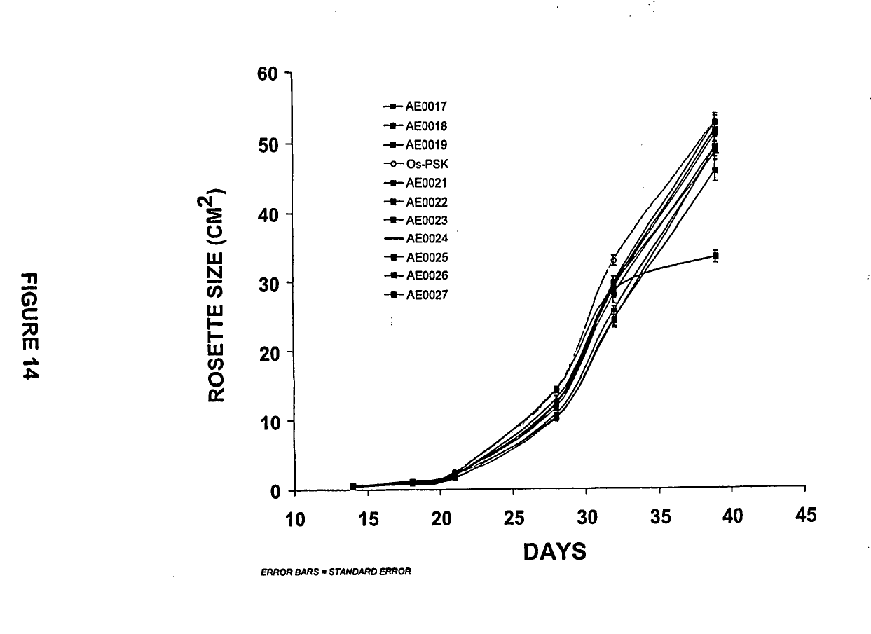

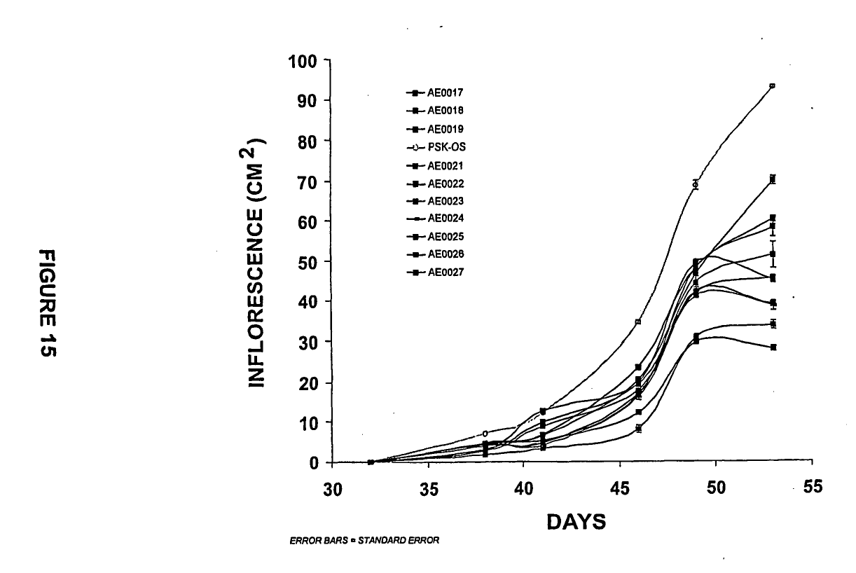

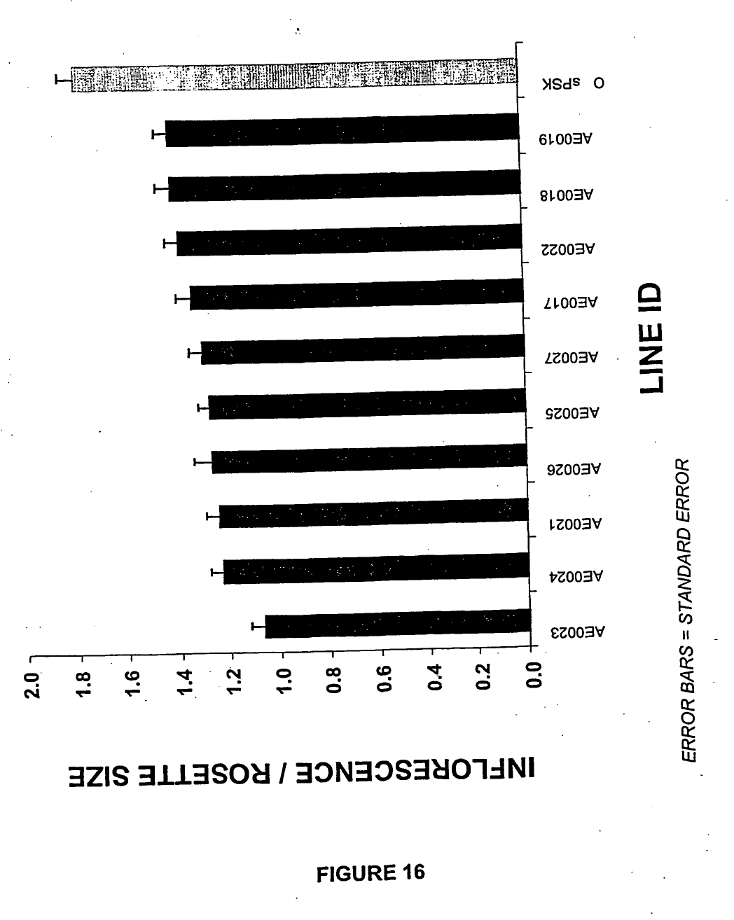

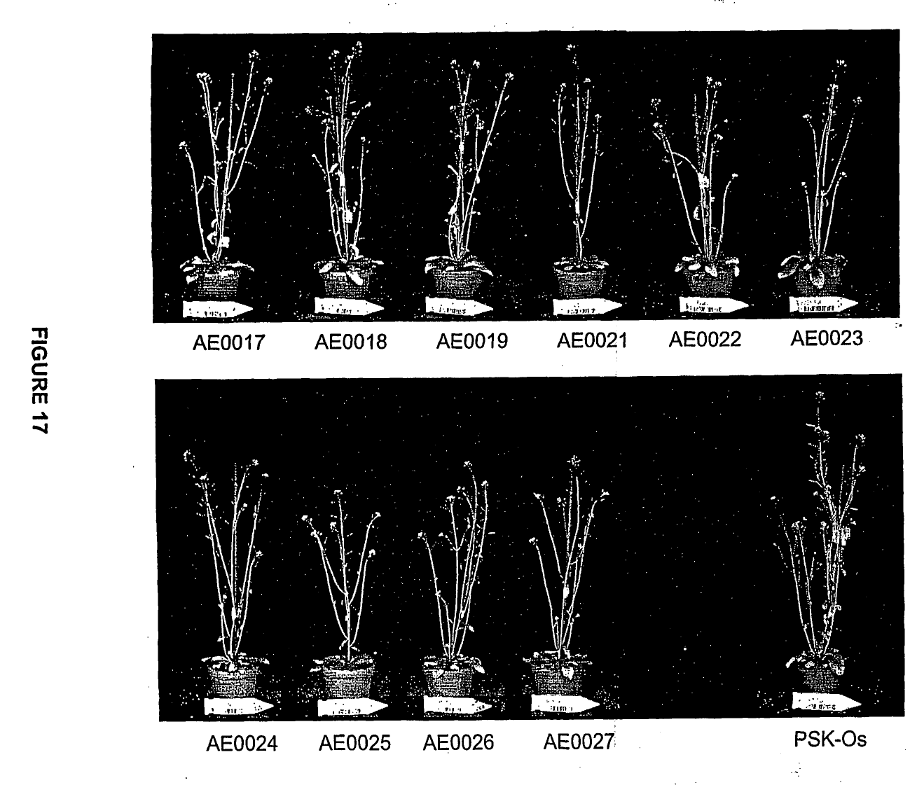

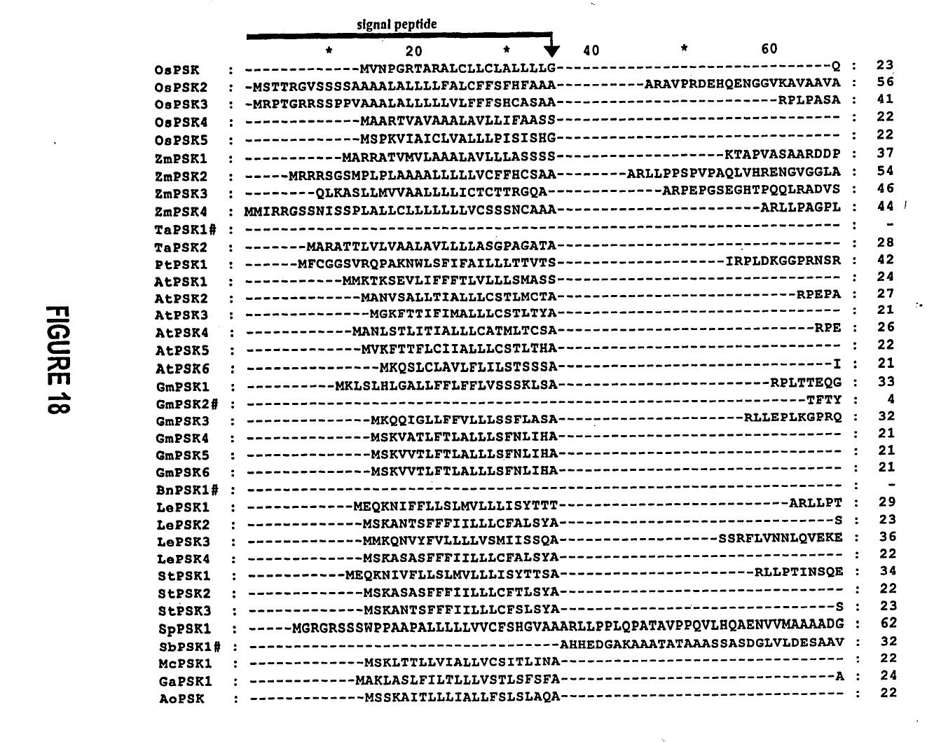

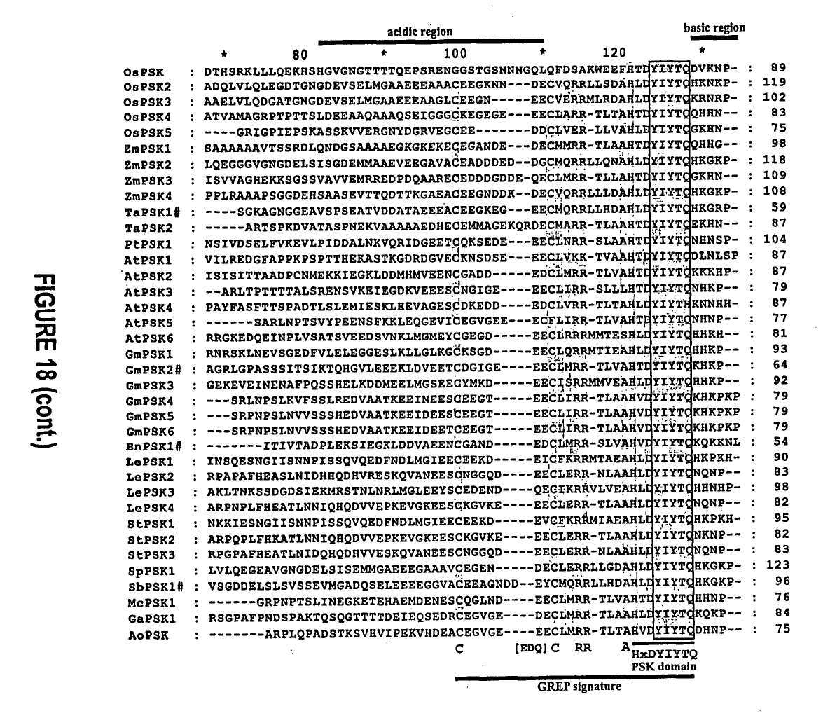

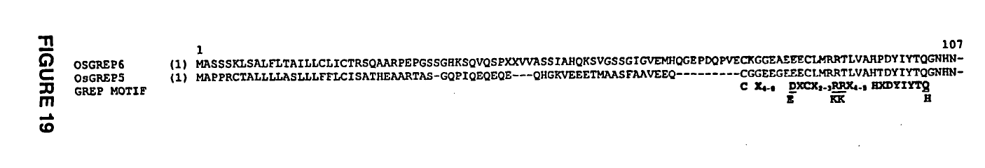

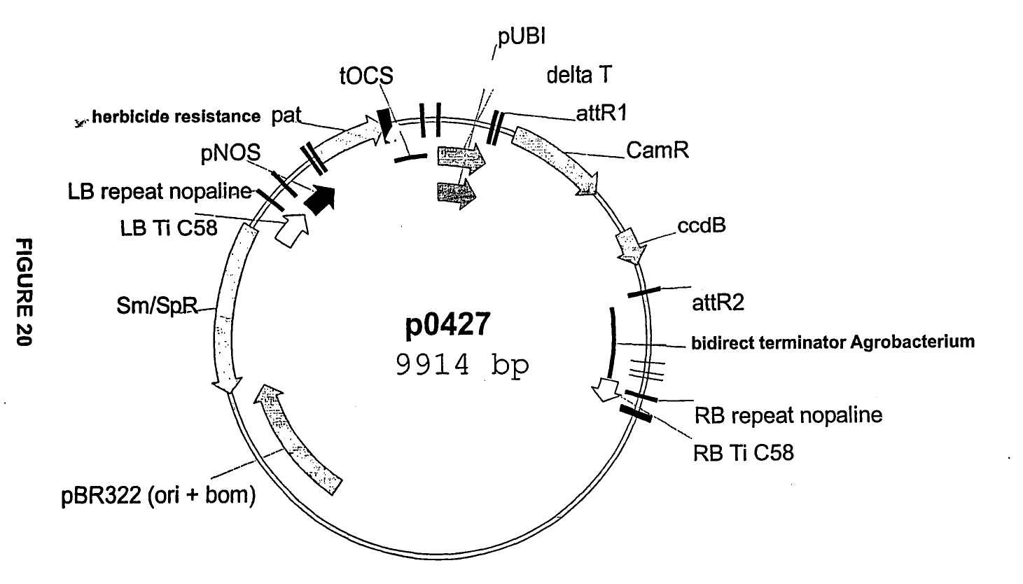

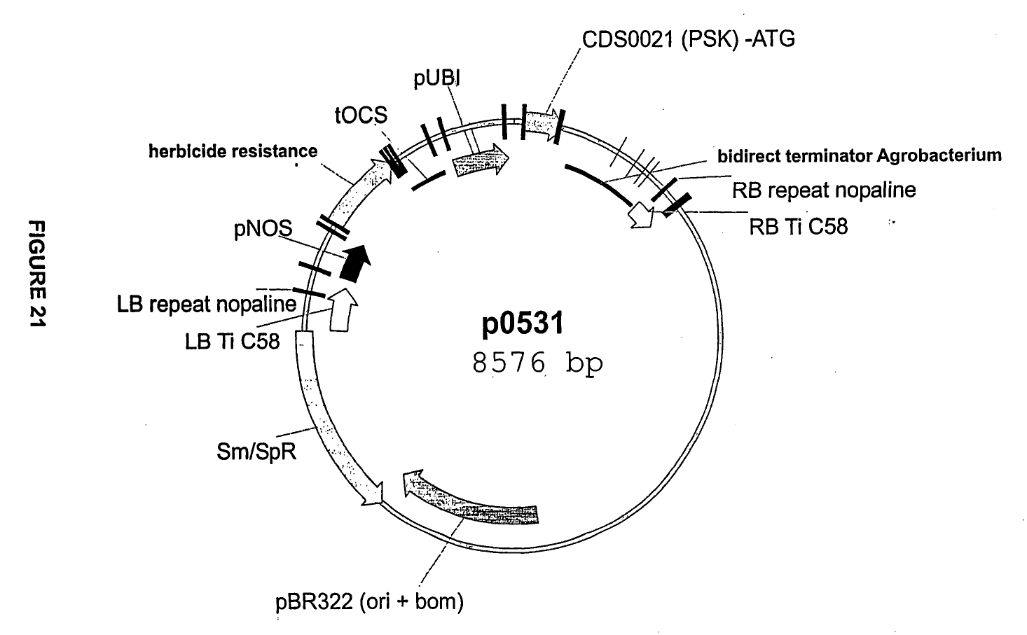

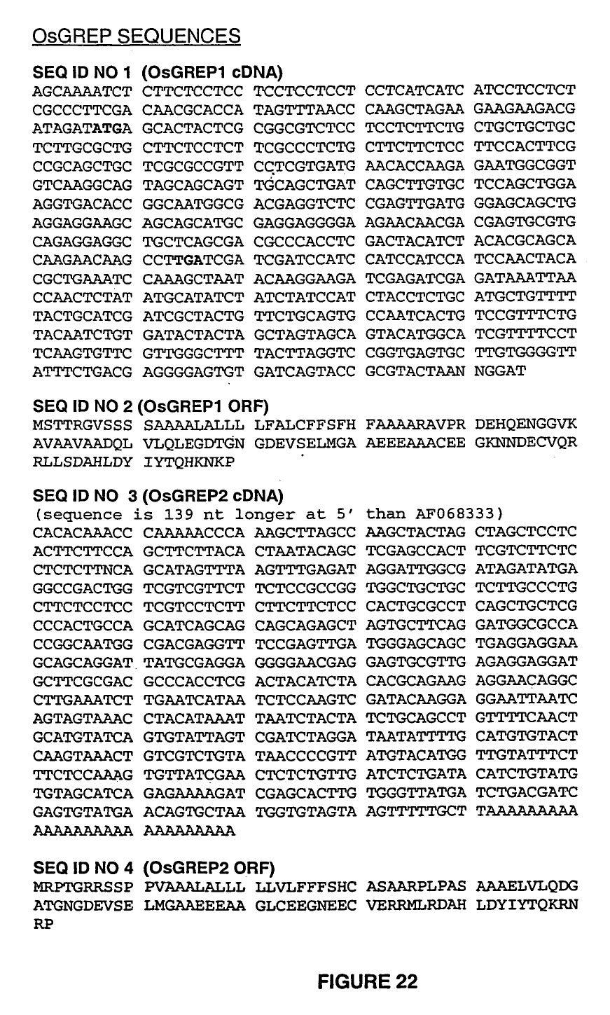

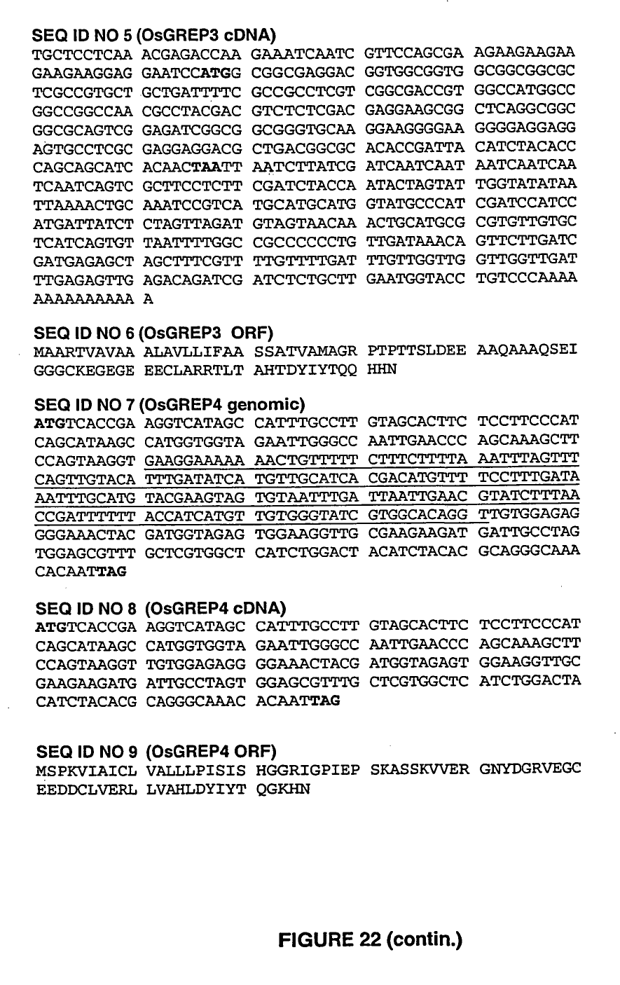

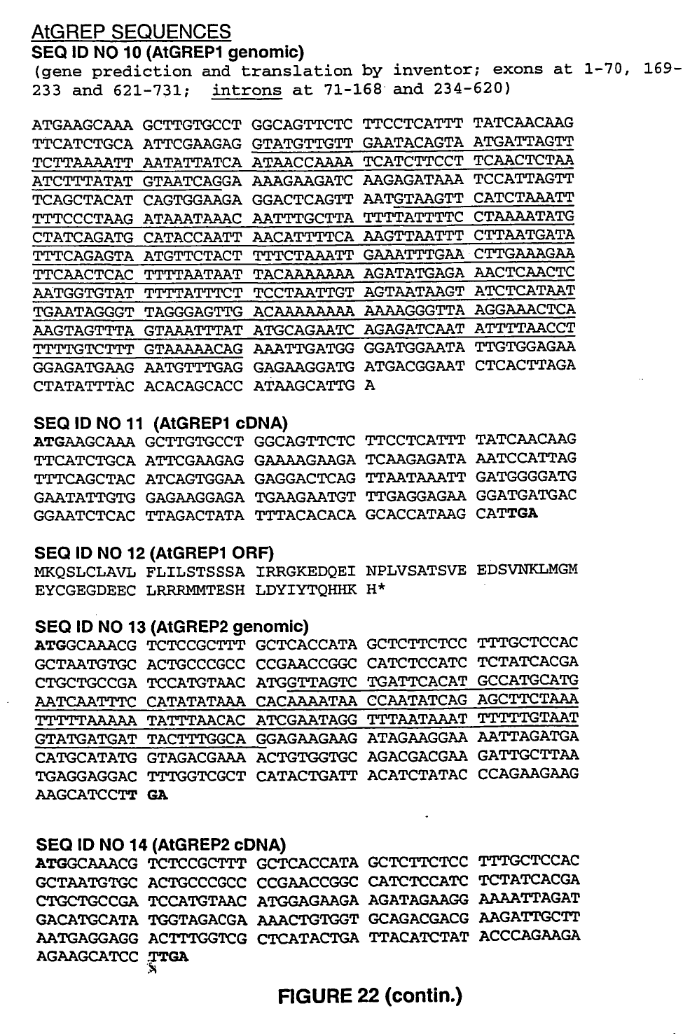

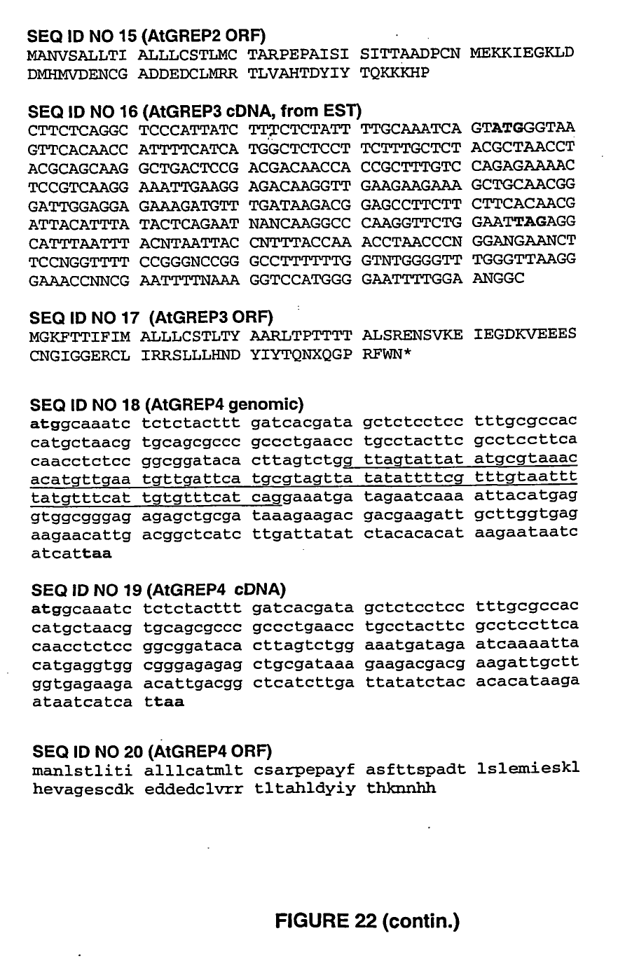

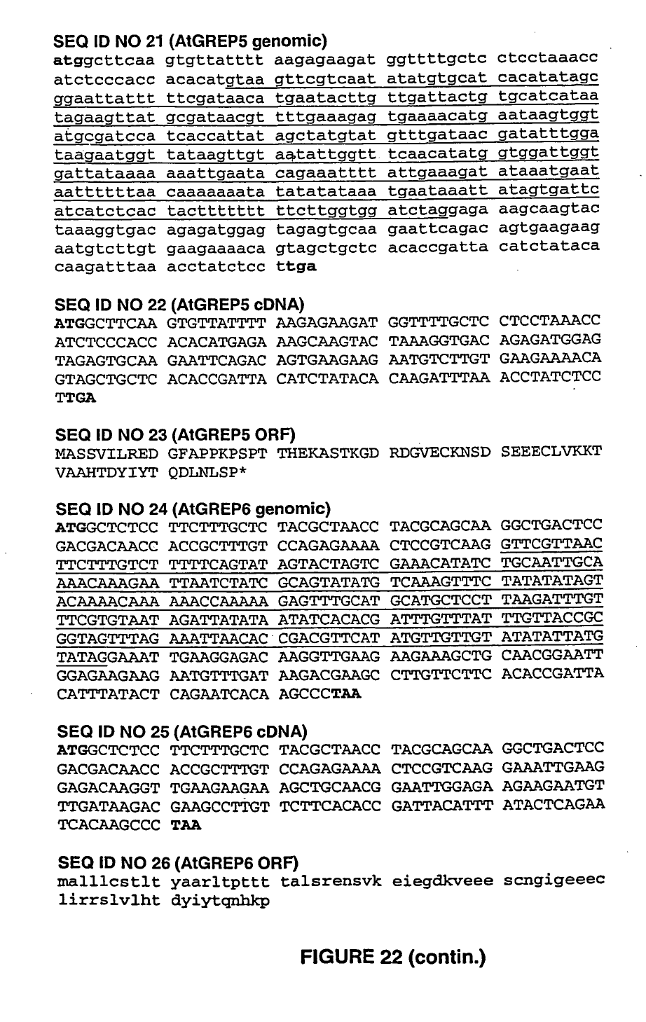

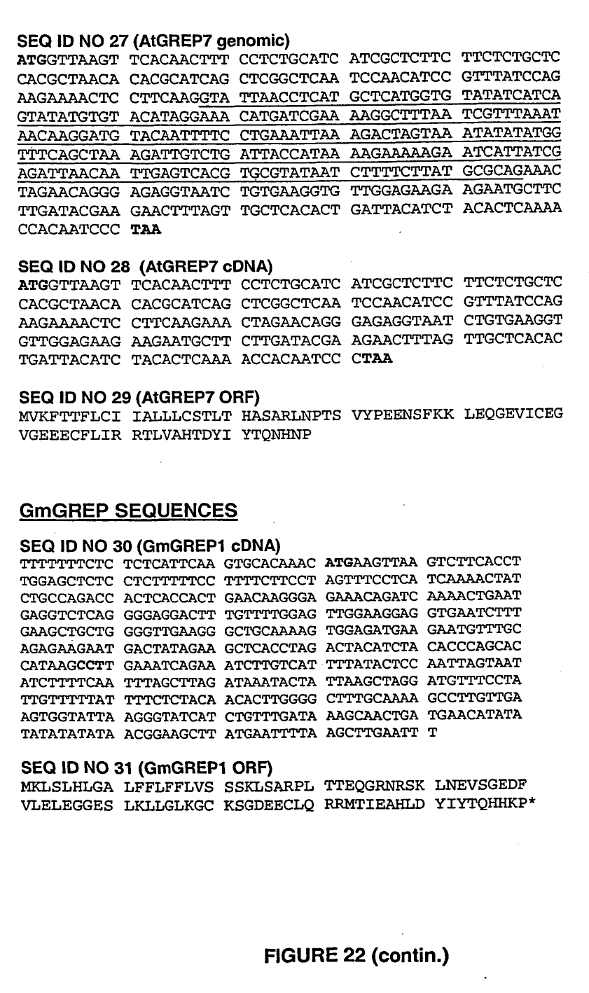

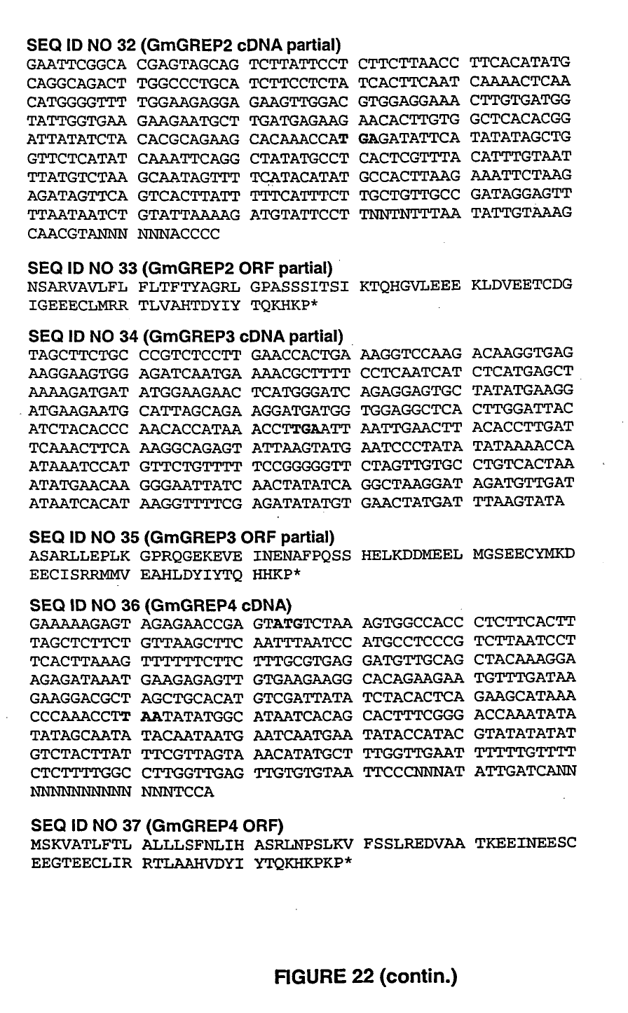

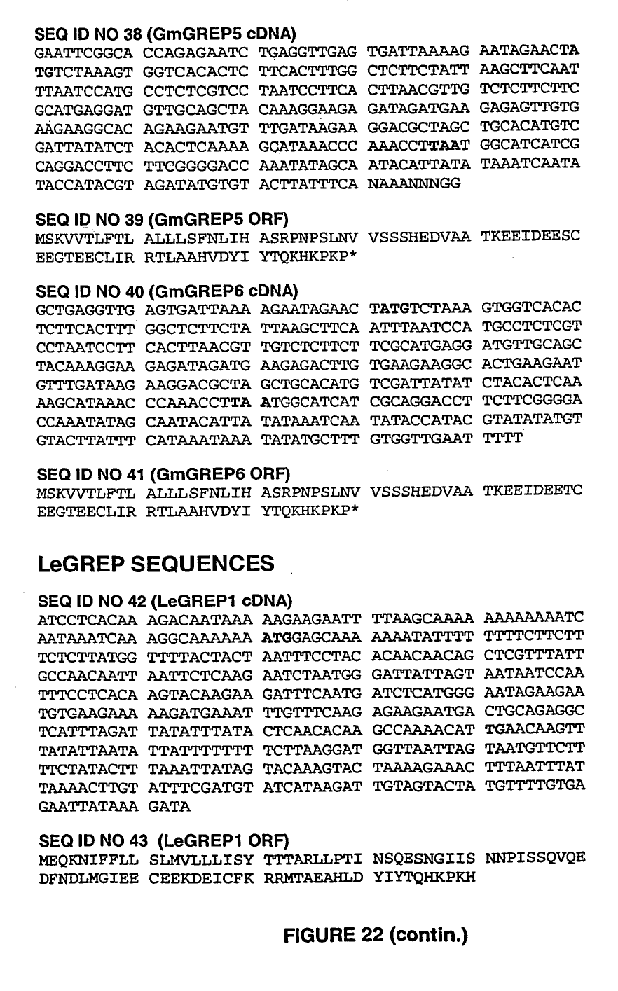

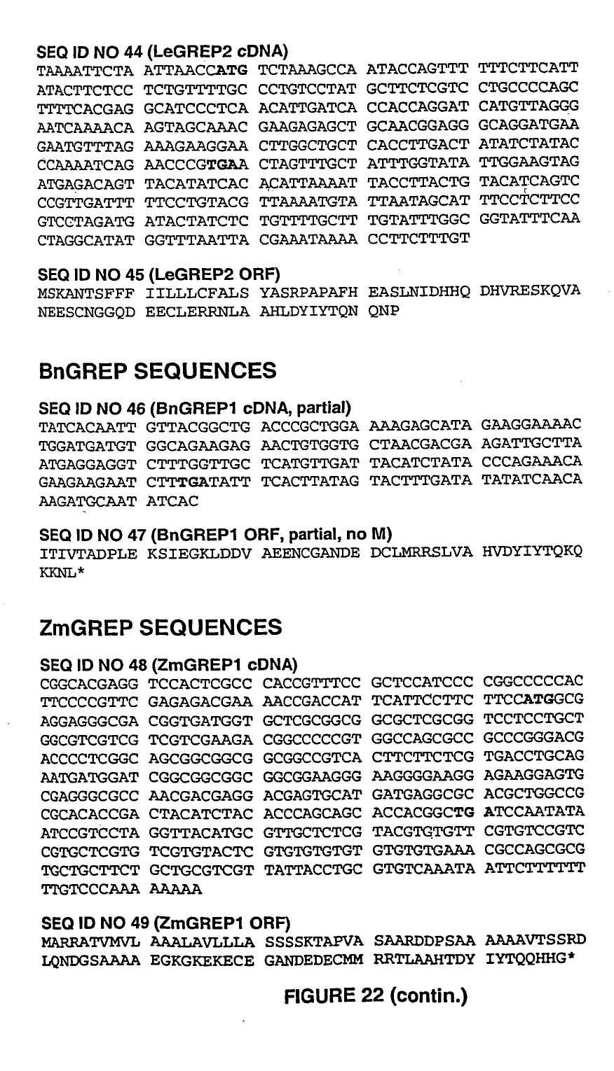

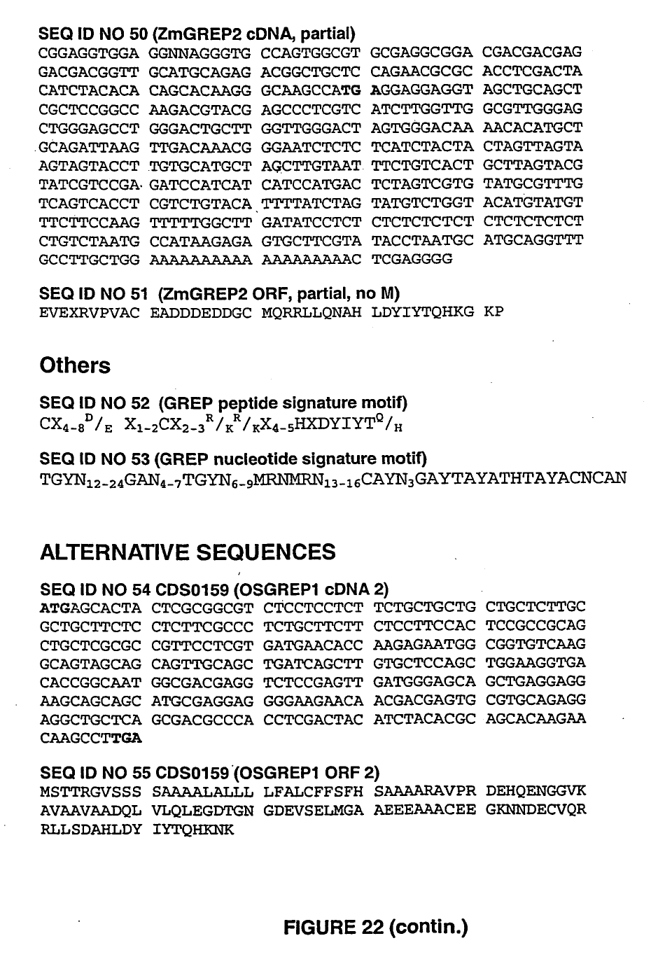

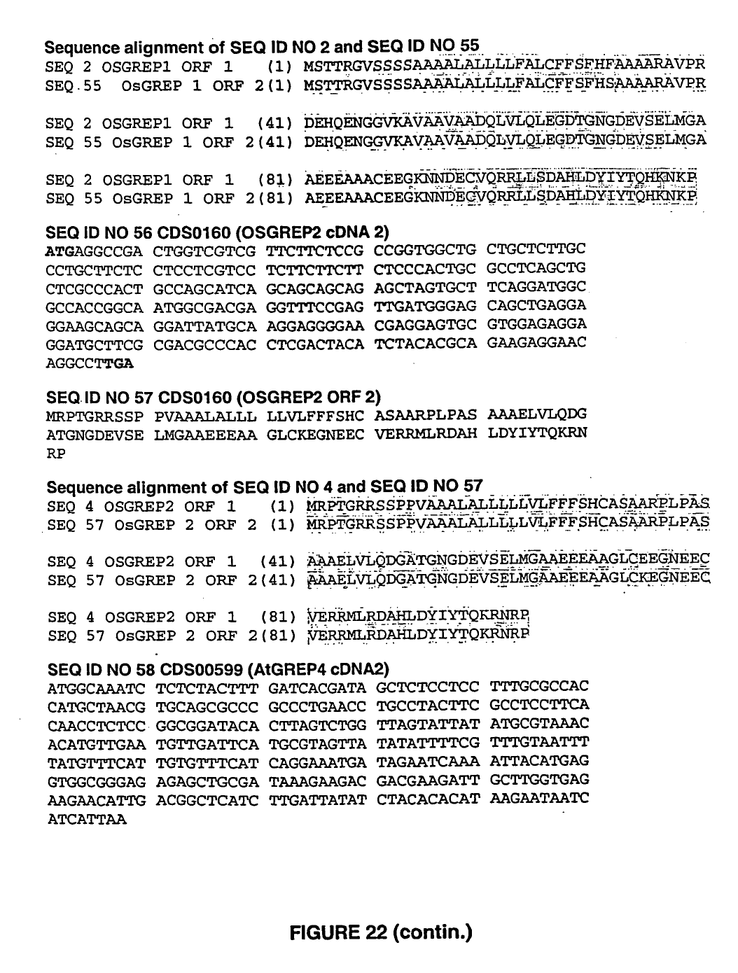

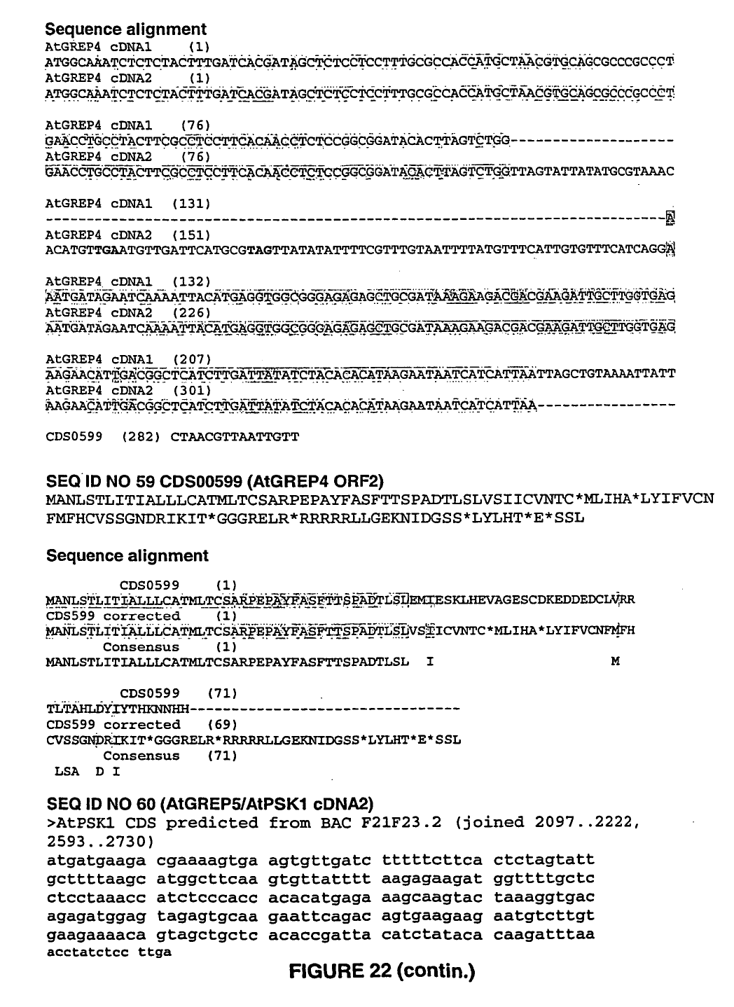

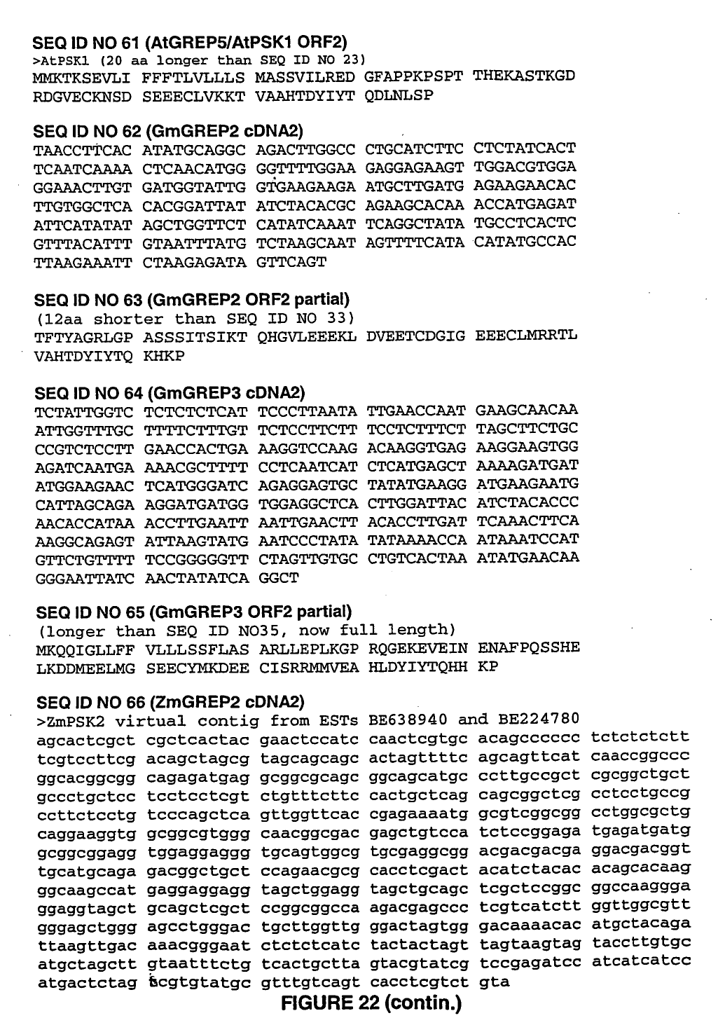

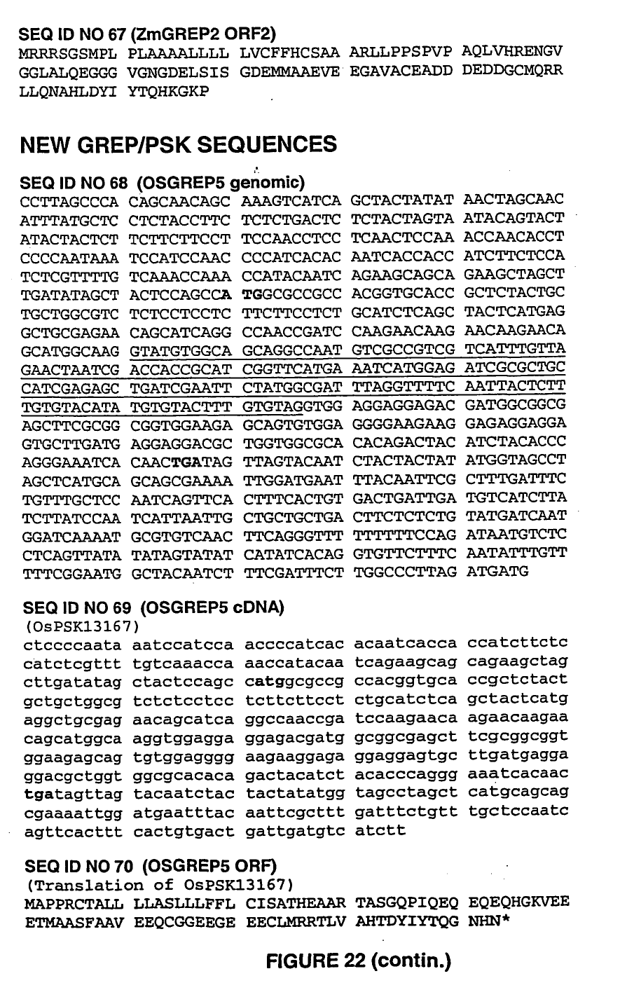

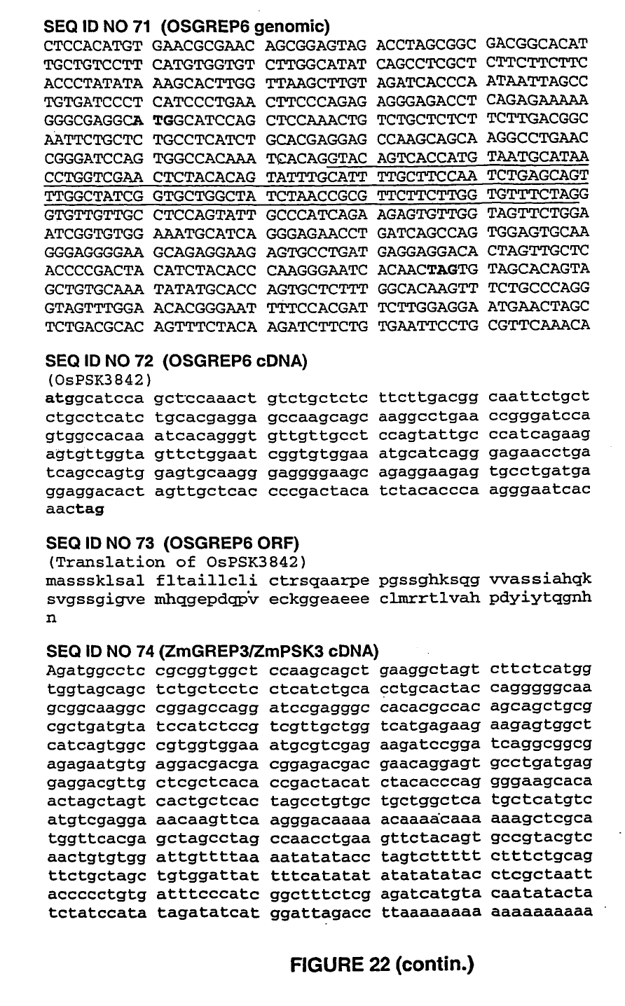

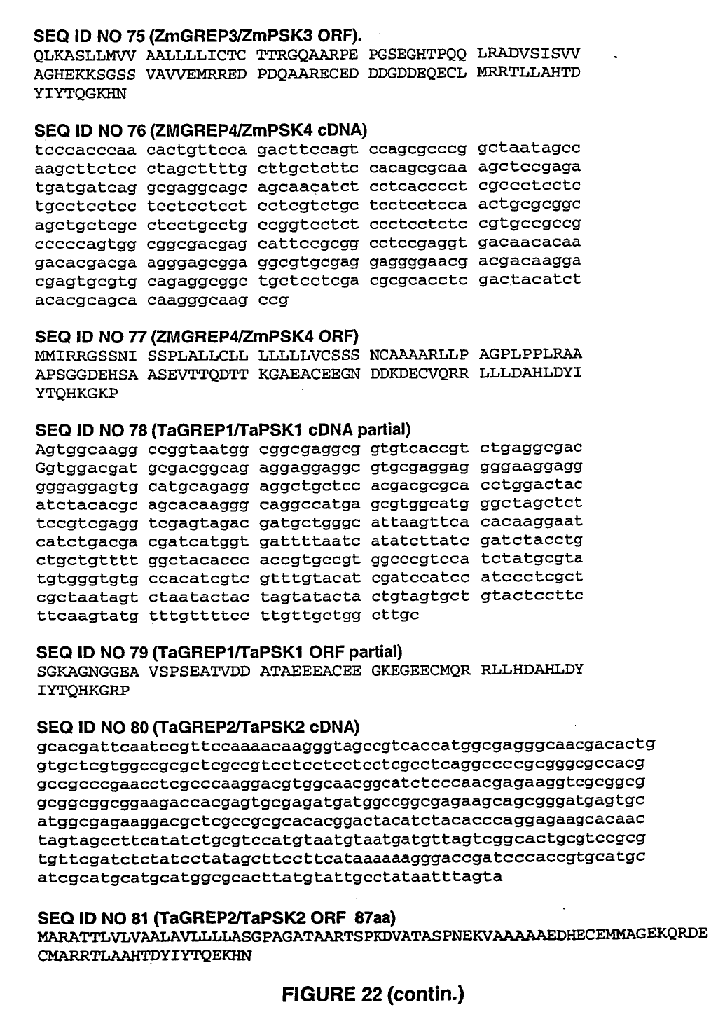

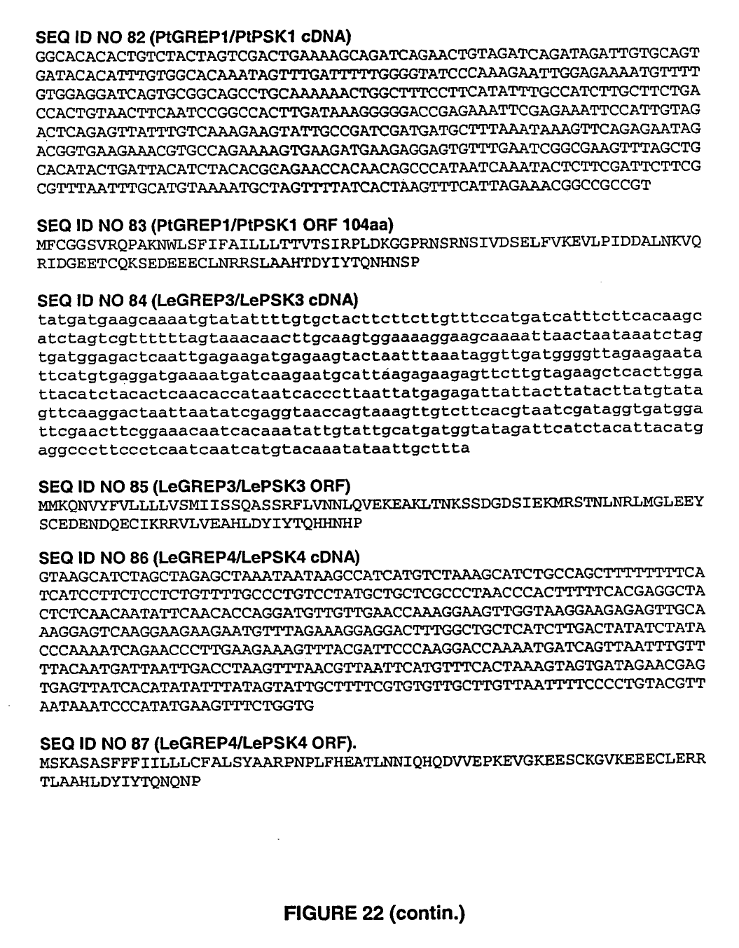

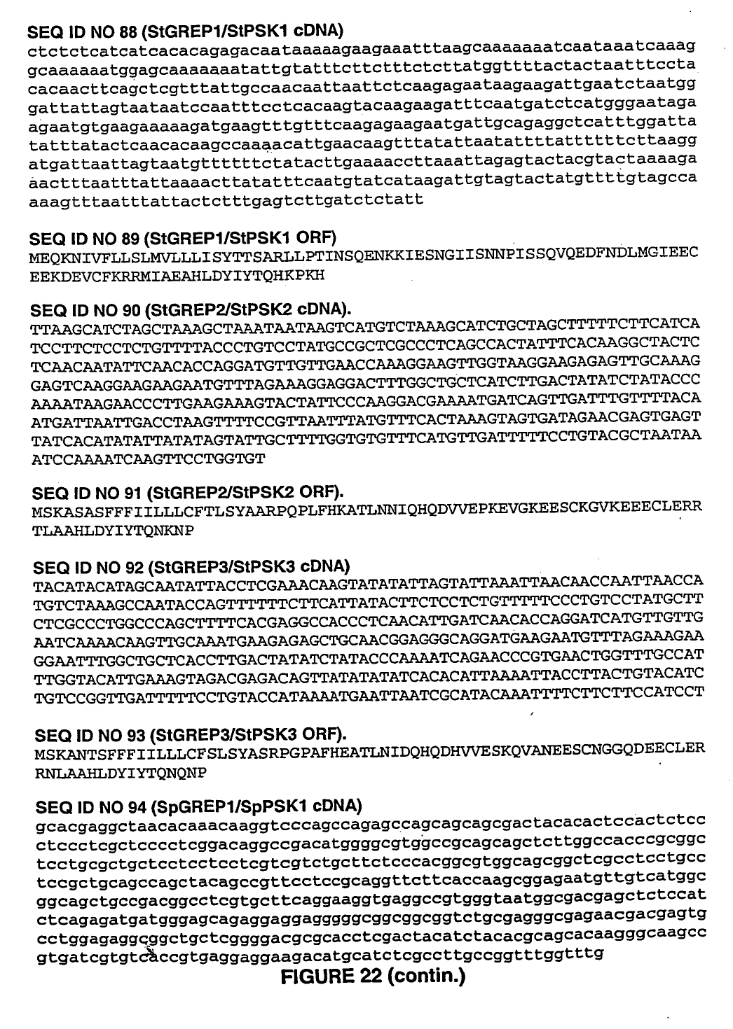

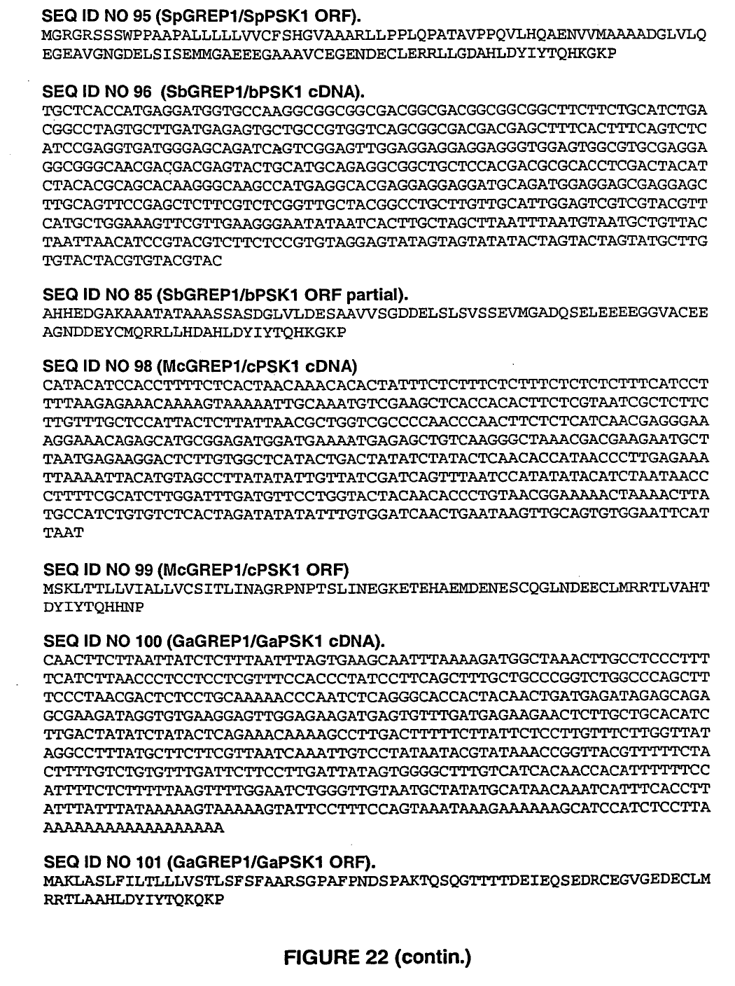

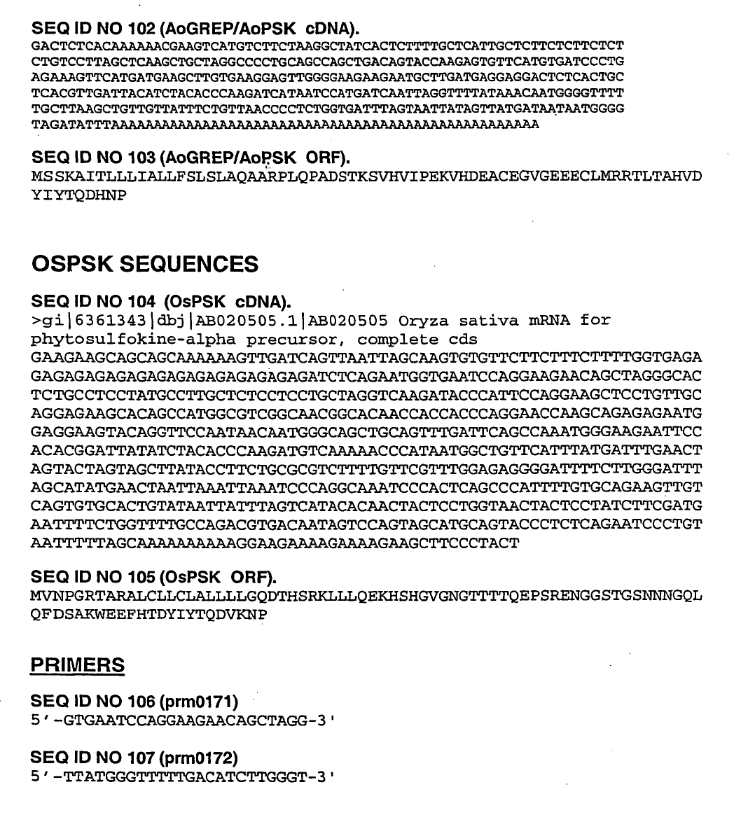

Database homology searches using OsGREP1 sequences combined with primary and secondary structure analysis of putative homologous proteins lead to the identification of homologous genes in rice, Arabidopsis, soybean, tomato, rape and maize (described in Examples 4, 0.5 and 6). An alignment of the full-length GREP peptide sequences is represented in FIG. 3 and the percentage identity between different GREPs is shown in FIG. 4. The overall peptide sequence identity between GREPs of different plant species is generally low but a core of conserved peptide sequences could be identified at the C-terminus of the GREP proteins. One aspect of the present invention involves this consensus sequence CX₄₋₈^{D}/_{E}X₁₋₂CX₂₋₃^{R}/_{K}^{R}/_{K}X₄₋₅HXDYIYT^{Q}/_{H} which is called the GREP signature motif and which can be used to identify and isolate genes encoding GREP proteins.

Despite the poor primary sequence conservation, many structural features are conserved among GREP growth regulating proteins that confirm their relationship (see Example 6). A further aspect of the invention is illustrated by the conserved primary and secondary structure characteristics of GREPs as disclosed in the present invention. All sequences start with an N-terminal hydrophobic peptide motif that corresponds to a putative signal sequence. The secondary structure of GREPs consists of three α-helices in the sequence following the hydrophobic signal region with a lower probability for a turn between the first and second helix of the postleader sequence. In addition, all GREPs contain an acidic region upstream of the GREP signature motif and a basic region at the C-terminus.

The YIYT sequence that is contained within the GREP signature motif corresponds to part of the pentapeptide backbone YIYTQ of the plant growth factor phytosulfokine-α (PSK-α). PSK-α is a sulfated pentapeptide hormone originally isolated from a cell culture medium (Matsubayashi & Sakagami, 1996; Matsubayashi et al. 1997). The cDNA that encodes PSK-α has recently been isolated from rice (Yang et al., 1999). This cDNA, termed OsPSK, encodes an 89-amino acid prepro-phytosulfokine that has a 22-amino acid hydrophobic region at its NH₂-terminus which resembles a cleavable leader peptide, similar to the GREP proteins disclosed in this invention. Genes homologous to OsPSK have been detected in other species including Arabidopsis thaliana, Asparagus officinalis, Daucus carota and Zinnia elegans and are considered unique genes in these plants.

The GREP proteins of the present invention and the OsPSK protein share the YIYT motif. However, the overall peptide sequence identity between the subject GREP proteins and OsPSK, is extremely low, ranging from 9 to 18%. More importantly, all GREP proteins share a second conserved motif in addition to the YIYT motif that is not present in OsPSK. The YIYT motif together with the upstream conserved region constitute the GREP signature motif CX₄₋₈^{D}/_{E}X₁₋₂CX₂₋₃^{R}/_{K}^{R}/_{K}X₄₋₅HXDYIYT^{Q}/_{H}. Because of the very low sequence conservation between OsPSK and the GREPs, database searching and hybridization experiments using OsPSK did not lead to the identification of the genes disclosed in this invention (see Example 6). In addition, the expression profile of the OsPSK gene is entirely different from that of the OsGREP1 gene disclosed in this invention. RNA gel blot analysis has shown that the OsPSK gene is highly expressed in in vitro cultured rice cells but not in intact plant tissues. OsPSK transcripts could be detected in rice seedling tissues through hybridization but only after amplification by RT-PCR (Yang et al, 2000). By contrast, the OsGREP1 gene of this invention is highly expressed in intact plant tissues such as for example roots as demonstrated by RNA gel blot analyses described in Example 7. Furthermore, OsGREP1 expression is induced in roots or internodes by a growth promoting treatment such as submergence (Example 7). These data indicate that OsGREP1 is involved in regulating growth responses in intact plants.

Accordingly, the present invention provides an isolated DNA sequence comprising a nucleotide sequence as given in SEQ ID NO 1 (OsGREP1 cDNA), or SEQ ID NO 10 (AtGREP1 genomic DNA) or SEQ ID NO 11 (AtGREP1 cDNA) with an amino acid sequence as given in SEQ ID NO 2 (OsGREP1), or SEQ ID NO 12 (AtGREP1), which encode a plant growth regulating protein. More specifically, said isolated DNA sequences provide novel genes, which encode a GREP plant growth regulating protein.

The nulceotide sequence of OsGREP1 was cloned and confirmed by sequence analysis. In FIG. 18 an alignment of the two alternative protein sequences is shown. The new sequence SEQ ID NO 54 has 1 nucleotide difference: C at position 92 instead of T. This nulceotide difference results in 1 amino acid substitution: S in SEQ ID NO 55 at position 31 instead of F. Unexpectedly, homologues were found in monocotyledonous and dicotyledonous plant species and which form gene families of GREP growth regulating protein encoding genes. GREP growth regulating proteins of different plant species can show low peptide sequence identity (15-25%). Even more surprising, the peptide sequences of all GREP growth regulating proteins have the same contiguous motif CX₄₋₈^{D}/_{E}X₁₋₂CX₂₋₃^{R}/_{K}^{R}/_{K}X₄₋₅HXDYIYT^{Q}/_{H} graphically represented in FIG. 3. Accordingly, the present invention also includes the GREP signature motif CX₄₋₅^{D}/_{E}X₁₋₂CX₂₋₃^{R}/_{K}^{R}/_{K}X₄₋₅HXDYIYT^{Q}/_{H} as given in SEQ ID NO 52. Therefore, in accordance with the present invention a previously unrecognized amino acid sequence motif has been identified in GREP growth regulating proteins which allows identification of said GREP growth regulating proteins. The identified signature motif is comprised in the carboxy-terminal part of the GREP growth regulating proteins. As described herein, overall sequence identity between GREP growth regulating proteins can be low, i.e. lower than 20% (see FIG. 4 and Example 6). This hampers the identification of novel GREP growth regulating protein-genes in plants. Therefore, the delineation of a conserved signature motif is of utmost importance to facilitate identification of said novel plant GREP growth regulating protein-genes and has been used in this invention to identify homologues of OsGREP1.

In addition, the presence or absence of said motif enables classification of GREP growth regulating proteins as distinct from the OsPSK protein. Genes encoding GREP growth regulating proteins can be isolated from plants based on the presence of this conserved sequence motif at the carboxy-terminus of the open reading frame. Finally, the conserved GREP motif as identified in the present invention may enable the delineation of a functionally important domain involved in protein processing, transport or other protein-protein interaction such as for example binding to specific receptors. Identification of such a domain can also facilitate the isolation of interacting proteins, the construction of dominant negative mutants and the design of gene silencing or cosuppression strategies. Accordingly, one embodiment of the current invention includes DNA sequences coding for a functional plant GREP growth regulating protein or a homologue thereof, which furthermore comprise DNA sequences encoding a peptide with the consensus sequence as given in SEQ ID NO 52 or a peptide that is at least 90%, preferably in the range of from about 90-95% and most preferably in the range of from about 95-100% identical thereto.

A related preferred embodiment of the current invention comprises an isolated nucleic acid encoding a GREP growth regulating protein as defined in this invention by the presence of the GREP signature motif and the structural characteristics of the corresponding protein.

Accordingly, the present invention also relates to nucleic acid molecules hybridizing with the above-described nucleic acid molecules and which differ in one or more positions in comparison with these as long as they encode a GREP growth regulating protein. GREP growth regulating proteins derived from other plants may be encoded by other DNA sequences which hybridize to the sequences disclosed in this invention under relaxed hybridization conditions. Examples of such non-stringent hybridization conditions are 4×SSC at 50° C. or hybridization with 30-40% formamide at 42° C. Such molecules comprise those which are fragments, analogues or derivatives of the Growth Regulating Protein of the invention and differ, for example, by way of amino acid and/or nucleotide deletion(s), insertion(s), substitution(s), addition(s) and/or recombination(s) or any other modification(s) known in the art, either alone or in combination from the above-described amino acid sequences or their underlying nucleotide sequence(s). Methods for introducing such modifications in the nucleic acid molecules according to the invention are well known to the person skilled in the art. The invention also relates to nucleic acid molecules, the sequence of which differs from the nucleotide sequence of any of the above-described nucleic acid molecules due to the degeneracy of the genetic code. All such fragments, analogues and derivatives of the protein of the invention are included within the scope of the present invention, as long as the essential characteristic immunological and/or biological properties as defined above remain unaffected in kind. That is, the nucleic acid molecules of the present invention include all nucleotide sequences encoding proteins or peptides which have at least a part of the primary structural conformation for one or more epitopes capable of reacting with antibodies to a Growth Regulating Protein which are encodable by a nucleic acid molecule as set forth above and which have comparable or identical characteristics as set forth in the definition of Growth Regulating Protein. Part of the invention are therefore also nucleic acid molecules encoding a polypeptide comprising at least a functional part of a GREP encoded by a nucleic acid sequence comprised in a nucleic acid molecule according to the invention. An example of this includes a polypeptide or a fragment thereof according to the invention, embedded in another amino acid sequence.

Preferably, a nucleic acid molecule which hybridizes to a nucleotide sequence as set forth in any one of SEQ ID NOs 1, 3, 5, 7, 8, 10, 11, 13, 14, 16, 18, 19, 22, 24, 25, 27, 28, 30, 32, 34, 36, 38, 40, 42, 44, 46, 48, 50, 53, 54, 56, 58, 60, 62, 64, 66, 68, 69, 71, 72, 74, 76, 78, 80, 82, 84, 86, 88, 90, 92, 94, 96, 98, 100 or 102, also comprising the consensus nucleotide sequence encoding the GREP signature motif (SEQ ID NO 52). Such a nucleotide sequence may be identified by hybridizing under stringent conditions using a degenerate probe having a nucleotide sequence as set forth in SEQ ID NO 53.

Preferably, such an isolated nucleic acid molecule may be inserted into a vector such as e.g., an expression vector. In an even more preferred embodiment, the isolated nucleic acid molecule is placed under the control of a promoter which functions in plants.

In a preferred embodiment, the nucleic acid molecules according to the invention are RNA or DNA molecules, preferably cDNA, genomic DNA or synthetically synthesized DNA or RNA molecules. Preferably, the nucleic acid molecule of the invention is derived from a plant, preferably from Oryza sativa or Arabidopsis thaliana. As discussed above, GREP proteins could also be identified in Brassica napus (rape), Zea Mays (corn), Glycine max (soybean) and Lycopersicon esculentum (tomato). Corresponding proteins displaying similar properties should therefore be present in other plants as well. Nucleic acid molecules of the invention can be obtained, e.g., by hybridization of the above-described nucleic acid molecules with a (sample of) nucleic acid molecule(s) of any source. Nucleic acid molecules hybridizing with the above-described nucleic acid molecules can in general be derived from any plant possessing such molecules, preferably form monocotyledonous or dicotyledonous plants, in particular from any plant of interest in agriculture, horticulture or wood culture, such as crop plants, namely those of the family Poaceae, any starch producing plants, such as potato, maniok, leguminous plants, oil producing plants, such as oilseed rape, linenseed, etc., plants using polypeptide as storage substances, such as soybean, plants using sucrose as storage substance, such as sugar beet of sugar cane, trees, ornamental plants etc. Preferably, the nucleic acid molecules according to the invention are derived from Oryza sativa or Arabidopsis thaliana. Nucleic acid molecules hybridizing to the above-described nucleic acid molecules can be isolated, e.g., from libraries, such as cDNA or genomic libraries by techniques well known in the art. For example, hybridizing nucleic acid molecules can be identified and isolated by using the above-described nucleic acid molecules or fragments thereof or complements thereof as probes to screen libraries by hybridizing with said molecules according to standard techniques. Possible is also the isolation of such nucleic acid molecules by applying the polymerase chain reaction (PCR) using as primers oligonucleotides derived form the above-described nucleic acid molecules.

Nucleic acid molecules which hybridize with any of the aforementioned nucleic acid molecules also include fragments, derivatives and allelic variants of the above-described nucleic acid molecules that encode a Growth Regulating Protein or an immunologically or functional fragment thereof and which comprise the signature GREP motif. Fragments are understood to be parts of nucleic acid molecules long enough to encode the described protein or a functional or immunologically active fragment thereof as defined above. Preferably, the functional fragment contains the signature GREP motif (SEQ ID NO 52) present in the carboxy-terminal part of the GREP proteins. Part of this motif corresponds to the plant mitogenic pentapeptide PSK-α.

Homology further means that the respective nucleic acid molecules or encoded proteins are functionally and/or structurally equivalent. The nucleic acid molecules that are homologous to the nucleic acid molecules described above and that are derivatives of said nucleic acid molecules are, for example, variations of said nucleic acid molecules which represent modifications having the same biological function, in particular encoding proteins with the same or substantially the same biological function. They may be naturally occurring variations, such as sequences from other plant varieties or species, or mutations. These mutations may occur naturally or may be obtained by mutagenesis techniques. The allelic variations may be naturally occurring allelic variants as well as synthetically produced or genetically engineered variants; see supra.

The proteins encoded by the various derivatives and variants of the above-described nucleic acid molecules share specific common characteristics, such as biological activity, molecular weight, immunological reactivity, conformation, etc., as well as physical properties, such as electrophoretic mobility, chromatographic behavior, sedimentation coefficients, pH optimum, temperature optimum, stability, solubility, spectroscopic properties, etc.

Examples of the different possible applications of the nucleic acid molecules according to the invention as well as molecules derived from them will be described in detail in the following.

Hence, in a further embodiment, the invention relates to nucleic acid molecules of at least 15 nucleotides in length hybridizing specifically with a nucleic acid molecule as described above or with a complementary strand thereof. Specific hybridization occurs preferably under stringent conditions and implies no or very little cross-hybridization with nucleotide sequences encoding no or substantially different proteins. Such nucleic acid molecules may be used as probes and/or for the control of gene expression. Nucleic acid probe technology is well known to those skilled in the art who will readily appreciate that such probes may vary in length. Preferred are nucleic acid probes of 16 to 35 nucleotides in length. Of course, it may also be appropriate to use nucleic acids of up to 100 and more nucleotides in length. The nucleic acid probes of the invention are useful for various applications. On the one hand, they may be used as PCR primers for amplification of nucleic acid sequences according to the invention. The design and use of said primers is known by the person skilled in the art. Preferably such amplification primers comprise a contiguous sequence of at least 6 nucleotides, in particular 13 nucleotides, preferably 15 to 25 nucleotides or more. Another application is the use as a hybridization probe to identify nucleic acid molecules hybridizing with a nucleic acid molecule of the invention by homology screening of genomic DNA or cDNA libraries. Nucleic acid molecules according to this preferred embodiment of the invention which are complementary to a nucleic acid molecule as described above may also be used for repression of expression of a GREP encoding gene, for example due to an antisense or triple helix effect or for the construction of appropriate ribozymes (see, e.g., EP-A1 0 291 533, EP-A1 0 321 201, EP-A2 0 360 257) which specifically cleave the (pre)-mRNA of a gene comprising a nucleic acid molecule of the invention or part thereof. Selection of appropriate target sites and corresponding ribozymes can be done as described, for example, in Steinecke, Ribozymes, Methods in Cell Biology 50, Galbraith et al. ads Academic Press, Inc. (1995), 449-460. In this aspect of the invention, a method of downregulating expression of a GREP in a plant comprises introducing into a plant cell a ribozyme targeted to a GREP transcript in the plant cell. Furthermore, the person skilled in the art is well aware that it is also possible to label such a nucleic acid probe with an appropriate marker for specific applications, such as for the detection of the presence of a nucleic acid molecule of the invention in a sample derived from a plant.

The above described nucleic acid molecules may either be DNA or RNA or a hybrid thereof. Furthermore, said nucleic acid molecule may contain, for example, thioester bonds and/or nucleotide analogues, commonly used in oligonucleotide anti-sense approaches. Said modifications may be useful for the stabilization of the nucleic acid molecule against endo- and/or exonucleases in the cell. Said nucleic acid molecules may be transcribed by an appropriate vector containing a chimeric gene which allows transcription of said nucleic acid molecule in the cell.

Furthermore, the so-called “peptide nucleic acid” (PNA) technique can be used for the detection or inhibition of the expression of a nucleic acid molecule of the invention. For example, the binding of PNAs to complementary as well as various single stranded RNA and DNA nucleic acid molecules can be systematically investigated using thermal denaturation and BIAcore surface-interaction techniques (Jensen, 1997). Furthermore, the nucleic acid molecules described above as well as PNAs derived therefrom can be used for detecting point mutations by hybridization with nucleic acids obtained from a sample with an affinity sensor, such as BIAcore; see Gotoh (1997). Hybridization based DNA screening on peptide nucleic acids (PNA) oligomer arrays are described in the prior art, for example in Weiler (1997). The synthesis of PNAs can be performed according to methods known in the art, for example, as described in Koch (1997); and Finn (1996). Further possible applications of such PNAs, for example as restriction enzymes or as templates for the synthesis of nucleic acid oligonucleotides are known to the person skilled in the art and are, for example, described in Veselkov (1996) and Bohler (1995).

The present invention also relates to vectors, particularly plasmids, cosmids, viruses, bacteriophages and other vectors used conventionally in genetic engineering that contain a nucleic acid molecule according to the invention. Methods which are well known to those skilled in the art can be used to construct various plasmids and vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1989). Alternatively, the nucleic acid molecules and vectors of the invention can be reconstituted into Jiposomes for delivery to target cells.

In a preferred embodiment the nucleic acid molecule present in the vector is linked to (a) control sequence(s) which allow the expression of the nucleic acid molecule in prokaryotic and/or eukaryotic cells. The vector of the invention is preferably an expression vector containing a screenable or scorable marker. This embodiment is particularly useful for simple and rapid screening of cells, tissues and organisms containing a vector of the invention.

The present invention furthermore relates to host cells comprising a vector as described above or a nucleic acid molecule according to the invention wherein the nucleic acid molecule is foreign to the host cell. The host cell can be any prokaryotic or eukaryotic cell, such as bacterial, insect, fungal, plant or animal cells. Preferred fungal cells are, for example, those of the genus Saccharomyces, in particular those of the species S. cerevisiae. Since the proteins of the present invention probably require extensive posttranslational processing and modification, particularly preferred host cells are plant cells.

Another subject of the invention is a method for the preparation of a GREP growth regulating protein or the active substance derived from a GREP growth regulating protein which comprises the cultivation of host cells according to the invention which, due to the presence of a vector or a nucleic acid molecule according to the invention, are able to express such a protein, under conditions which allow expression of the protein and recovering of the so-produced protein from the culture. For the preparation of the active substance derived from a GREP growth regulating protein, particularly preferred host cells are plant cells since plant cells will be able to ensure proper maturation and processing of the GREP proteins into a functional product.

The present invention furthermore relates to GREP growth regulating proteins encoded by the nucleic acid molecules according to the invention or produced or obtained by the above-described methods, and to functional and/or immunologically active fragments of such GREP proteins. The proteins and polypeptides of the present invention are not necessarily translated from a designated nucleic acid sequence; the polypeptides may be generated in any manner, including for example, chemical synthesis, or expression of a recombinant expression system, or isolation from a suitable viral system. The polypeptides may include one or more analogues of amino acids, phosphorylated amino acids or unnatural amino acids. Methods of inserting analogues of amino acids into a sequence are known in the art. The polypeptides may also include one or more labels, which are known to those skilled in the art. In this context, it is also understood that the proteins according to the invention may be further modified by conventional methods known in the art. By providing the proteins according to the present invention it is also possible to determine fragments which retain biological activity, for example, the mature, processed form. This allows the construction of chimeric proteins and peptides comprising an amino sequence derived from the protein of the invention, which is crucial for its binding activity and other functional amino acid sequences, e.g. GUS marker gene (Jefferson, 1987). The other functional amino acid sequences may be either physically linked by, e.g., chemical means to the proteins of the invention or may be fused by recombinant DNA techniques well known in the art.

Furthermore, folding simulations and computer redesign of structural motifs of the protein of the invention can be performed using appropriate computer programs (Olszewski, 1996; Hoffman, 1995). Computer modelling of protein folding can be used for the conformational and energetic analysis of detailed peptide and protein models (Monge, 1995; Renouf, 1995). In particular, the appropriate programs can be used for the identification of interactive sites of the GREP growth regulating proteins, its ligand or other interacting proteins by computer assistant searches for complementary peptide sequences (Fassina, 1994). Further appropriate computer systems for the design of protein and peptides are described in the prior art, for example in Berry (1994); Wodak (1987); Pabo (1986). The results obtained from the above-described computer analysis can be used for, e.g., the preparation of peptidomimetics of the protein of the invention or fragments thereof. Such pseudopeptide analogues of the natural amino acid sequence of the protein may very efficiently mimic the parent protein (Benkirane, 1996). For example, incorporation of easily available achiral Ω-amino acid residues into a protein of the invention or a fragment thereof results in the substitution of amide bonds by polymethylene units of an aliphatic chain, thereby providing a convenient strategy for constructing a peptidomimetic (Banerjee, 1996). Superactive peptidomimetic analogues of small peptide hormones in other systems are described in the prior art (Zhang, 1996). Appropriate peptidomimetics of the protein of the present invention can also be identified by the synthesis of peptidomimetic combinatorial libraries through successive amide alkylation and testing the resulting compounds, e.g., for their binding, kinase inhibitory and/or immunological properties. Methods for the generation and use of peptidomimetic combinatorial libraries are described in the prior art, for example in Ostresh (1996) and Domer (1996).

Furthermore, a three-dimensional and/or crystallographic structure of the protein of the invention can be used for the design of peptidomimetic inhibitors of the biological activity of the protein of the invention (Rose, 1996; Rutenber, 1996).

Furthermore, the present invention relates to antibodies specifically recognizing a GREP protein according to the invention or parts thereof, i.e. specific fragments or epitopes, of such a protein. The antibodies of the invention can be used to identify and isolate other GREPs in different plants. These antibodies can be monoclonal antibodies, polyclonal antibodies or synthetic antibodies as well as fragments of antibodies, such as Fab, Fv or scFv fragments etc. Monoclonal antibodies can be prepared, for example, by the techniques as originally described in Köhler and Milstein (1975), and Galfré (1981), where mouse myeloma cells are fused to spleen cells derived from immunized mammals. Furthermore, antibodies or fragments thereof to the aforementioned peptides can be obtained by using methods which are described, e.g., in Harlow and Lane “Antibodies, A Laboratory Manual”, CSH Press, Cold Spring Harbor (1988). These antibodies can be used, for example, for the immunoprecipitation and immunolocalization of proteins according to the invention as well as for the monitoring of the synthesis of such proteins, for example, in recombinant organisms, and for the identification of compounds interacting with the protein according to the invention. For example, surface plasmon resonance as employed in the BIAcore system can be used to increase the efficiency of phage antibodies selections, yielding a high increment of affinity from a single library of phage antibodies which bind to an epitope of the protein of the invention (Schier, 1996; Malmborg, 1995). In many cases, the binding phenomena of antibodies to antigens is equivalent to other ligand/anti-ligand binding.

Modulation of the expression of a polypeptide encoded by a nucleotide sequence according to the invention has an advantageous influence on plant growth characteristics, for example on root growth in case of OsGREP1, and as a result thereof on the total makeup of the plant-concerned or parts thereof. GREPs or the active substance derived thereof is active as a plant growth regulator and functions in a signal transduction pathway that ultimately leads to altered plant growth characteristics. The activity of a GREP in a plant cell is influenced by manipulation of the gene according to the invention. Transformed plants can be made to overproduce the nucleotide sequences according to the invention. Such an overexpression of the new gene(s), proteins or inactivated variants thereof, will either positively or negatively have an effect on an aspect of plant cell growth. Methods to modify the expression levels and/or the activity are known to persons skilled in the art and include for instance overexpression, co-suppression, the use of ribozymes, sense and anti-sense strategies, gene silencing approaches. Hence, the nucleic acid molecules according to the invention are in particular useful for the genetic manipulation of plant cells in order to modify the growth characteristics of plants and to obtain plants with modified, preferably with improved or useful phenotypes. Similarly, the invention can also be used to modulate the growth of cells or tissues, preferentially plant cells, in in vitro cultures. Thus, the present invention provides for a method for the production of transgenic plants, plant cells or plant tissues comprising the introduction of a nucleic acid molecule or vector of the invention into the genome of said plant, plant cell or plant tissue.

For the expression of the nucleic acid molecules according to the invention in sense or antisense orientation in plant cells, the molecules are placed under the control of regulatory elements, which ensure the expression in plant cells. These regulatory elements may be heterologous or homologous with respect to the nucleic acid molecule to be expressed as well with respect to the plant species to be transformed. In general, such regulatory elements comprise a promoter active in plant cells, i.e., a promoter which functions in plant cells.

To obtain uniform expression of a GREP in all plant cells, constitutive promoters are used, such as those listed in Table 1. When GREP proteins are expressed constitutively, regeneration of shoots from transgenic rice callus may be more difficult due to a perturbed hormonal balance in the callus that prevents shoot regeneration. To enable the production of transgenic plants with modified growth characteristics, the expression of the nucleic acid molecule encoding a GREP is preferably controlled by the use of tissue-specific, cell type-specific, tissue-preferred or inducible promoters. Promoters which are specifically active in tubers of potatoes or in seeds of different plants species, such as maize, Vicia, wheat, barley etc., may also be used in accordance with the present invention. Inducible promoters may be used in order to be able to exactly control expression. Examples of inducible promoters include the promoters of genes encoding heat shock proteins. Also microspore-specific regulatory elements and their uses have been described (WO96/16182). Furthermore, the chemically inducible Test-system may be employed (Gatz, 1991). Further suitable promoters are known to those skilled in the art, many of which are listed in Table 2. The regulatory elements may further comprise transcriptional and/or translational enhancers functional in plant cells. Furthermore, the regulatory elements may include transcription termination signals and polyadenylation signals which lead to the addition of a poly(A) tail to the transcript which may improve its stability and translation.

A subject nucleic acid molecule may have its coding sequences modified in such a way that the corresponding protein is transported to any desired compartment of the plant cell. Examples of such compartments include the nucleus, endoplasmatic reticulum, the vacuole, the mitochondria, the plastids, the apoplast, the cytoplasm etc. Since GREPs have a putative aminoterminal hydrophobic leader sequence for targeting to the secretory pathway, corresponding signal sequences are preferred to direct the protein of the invention to the same compartment. Methods of incorporating such modifications and signal sequences into a nucleic acid molecule in order to ensure localization in a desired compartment are well known to the person skilled in the art.

The cDNA's of the present invention provide sufficient signaling sequences to set the active protein free in the host cell and to process it and localize it in the secretory system, so that it can function also outside the cell where it is produced. In this way the cNDA can exert also its effect on other host cells, in other plant tissues etc.

Specific characteristics of transgenic plants overexpressing GRP or PSK encoding nucleic acids are (1) cell proliferation induced in early stages of seeds development (2) improved plant growth and yiels (3) early vigor (4) increased inflorescence etc.

In general, the plants which may be modified according to the invention and which either show overexpression of a protein according to the invention or a reduction of the synthesis of such a protein can be derived from any desired plant species. They can be monocotyledonous plants or dicotyledonous plants. Preferably they belong to plant species of interest in agriculture, wood culture or horticulture interest, such as crop plants (e.g. maize, rice, barley, wheat, rye, oats etc.), potatoes, oil producing plants (e.g. oilseed rape, sunflower, peanut, soy bean, etc.), cotton, sugar beet, sugar cane, leguminous plants (e.g. beans, peas etc.), wood producing plants, preferably trees, etc.

Thus, the present invention relates also to transgenic plant cells which contain stably integrated into the genome a nucleic acid molecule according to the invention linked to regulatory elements which allow for expression of the nucleic acid molecule in plant cells and wherein the nucleic acid molecule is foreign to the transgenic plant cell. Alternatively, a plant cell having (a) nucleic acid molecule(s) encoding a Growth Regulating Protein present in its genome can be used and modified such that said plant cell expresses the endogenous gene(s) corresponding to these nucleic acid molecules under the control of an heterologous promoter and/or enhancer elements. The introduction of the heterologous promoter and mentioned elements which do not naturally control the expression of a nucleic acid molecule encoding the above described protein using, e.g., gene targeting vectors can be done according to standard methods, see supra and, e.g., Hayashi, 1992; Fritze and Walden, 1995) or transposon tagging (Chandlee, 1990). Suitable promoters and other regulatory elements such as enhancers include those mentioned hereinbefore.

The presence and expression of the nucleic acid molecule in the transgenic plant cells leads to the synthesis of a Growth Regulating Protein and leads to physiological and phenotypic changes in plants containing such cells. Thus, the present invention also relates to transgenic plants and plant tissue comprising transgenic plant cells according to the invention. Due to the (over) expression of a Growth Regulating Protein of the invention, e.g., at developmental stages and/or in plant tissue in which they do not naturally occur, these transgenic plants can show various physiological, developmental and/or morphological modifications in comparison to wild-type plants. For example, these transgenic plants can display altered growth characteristics.

Therefore, part of this invention is the use of GREPs and the encoding DNA sequences to modulate growth in plant cells, plant tissues, plant organs and/or whole plants. In one embodiment, there is provided a method to influence the activity of GREPs in a plant cell by transforming the plant cell with a subject nucleic acid molecule and/or manipulation of the expression of said molecule. More in particular using a nucleic acid molecule according to the invention, the disruption of plant cell growth can be accomplished by interfering in the activity of GREPs or their interactors.

Hence, the invention also relates to a transgenic plant cell which contains (stably integrated into the genome) a nucleic acid molecule according to the invention or part thereof, wherein the transcription and/or expression of the nucleic acid molecule or part thereof leads to reduction of the synthesis of a Growth Regulating Protein. In a preferred embodiment, the reduction is achieved by an anti-sense, sense, ribozyme, co-suppression and/or dominant mutant effect.

In another aspect of the invention, transgenic plant cells with a reduced level of a subject GREP protein as described above are provided. Techniques how to achieve this are well known to the person skilled in the art. These include, for example, the expression of antisense-RNA, ribozymes, of molecules which combine antisense and ribozyme functions and/or of molecules which provide for a co-suppression effect When using the antisense approach for reduction of the amount of GREP in plant cells, the nucleic acid molecule encoding the antisense-RNA is preferably of homologous origin with respect to the plant species used for transformation. However, it is also possible to use nucleic acid molecules which display a high degree of homology to endogenously occurring nucleic acid molecules encoding a GREP. In this case the homology is preferably higher than 80%, particularly higher than 90% and still more preferably higher than 95%.

The reduction of the synthesis of A protein according to the invention in the transgenic plant cells can result in an alteration in, e.g., cell growth. In transgenic plants comprising such cells this can lead to various physiological, developmental and/or morphological changes.

Thus, the present invention also relates to transgenic plants comprising the above-described transgenic plant cells. These may show, for example, reduced or enhanced growth characteristics.

The present invention also relates to cultured plant tissues comprising transgenic plant cells as described above which either show overexpression of a protein according to the invention or a reduction in synthesis of such a protein.

Any transformed plant obtained according to the invention can be used in a conventional breeding scheme or in in vitro plant propagation to produce more transformed plants with the same characteristics and/or can be used to introduce the same characteristic in other varieties of the same or related species. Such plants are also part of the invention. Seeds obtained from the transformed plants genetically also contain the same characteristic and are part of the invention. As mentioned before, the present invention is in principle applicable to any plant and crop that can be transformed with any of the transformation method known to those skilled in the art and includes for instance corn, wheat, barley, rice, oilseed crops, cotton, tree species, sugar beet, cassaya, tomato, potato, and numerous other vegetables and fruits.

In yet another aspect, the invention also relates to harvestable parts and to propagation material of the transgenic plants according to the invention which either contain transgenic plant cells expressing a nucleic acid molecule according to the invention or which contain cells which show a reduced level of the described protein. Harvestable parts can be in principle any useful part of a plant, for example, flowers, pollen, seedlings, tubers, leaves, stems, fruit, seeds, roots etc. Propagation material includes, for example, seeds, fruits, cuttings, seedlings, tubers, rootstocks etc.

As mentioned above, the OsGREPs of the invention display distinct expression patterns in plants and in cell suspension cultures. Thus, the regulatory sequences that naturally drive the expression of these GREPs are useful for the expression of heterologous DNA sequences in certain plant tissues and/or at different developmental stages in plant development. Accordingly, in a further aspect the present invention relates to a regulatory sequence of a promoter which naturally regulates the expression of a nucleic acid molecule of the subject invention or of a nucleic acid molecule homologous to a nucleic acid molecule of the invention. The expression pattern of the OsGREP genes has been studied in detail in accordance with the present invention and is summarized in Example 7. With methods well known in the art it is possible to isolate the regulatory sequences of the promoters that naturally regulate the expression of the above-described DNA sequences. For example, using the OsGREP genes as probes a genomic library consisting of plant genomic DNA cloned into phage or bacterial vectors can be screened by a person skilled in the art. Such a library consists e.g. of genomic DNA prepared from seedlings, fractionized in fragments ranging from 5 kb to 50 kb, cloned into the lambda GEM11 (Promega) phages. Phages hybridizing with the probes can be purified. From the purified phages DNA can be extracted and sequenced. Having isolated the genomic sequences corresponding to the genes encoding the above-described GREPs, it is possible to fuse heterologous DNA sequences to these promoters or their regulatory sequences via transcriptional or translational fusions well known to the person skilled in the art. In order to identify the regulatory sequences and specific elements of the GREP genes, 5′-upstream genomic fragments can be cloned in front of marker genes such as luc, gfp or the GUS coding region and the resulting chimeric genes can be introduced by means of Agrobacterium tumefaciens mediated gene transfer into plants or transfected into plant cells or plant tissue for transient expression. The expression pattern observed in the transgenic plants or transfected plant cells containing the marker gene under the control of the regulatory sequences of the invention reveal the boundaries of the promoter and its regulatory sequences.

It is also immediately evident to the person skilled in the art that further regulatory elements may be added to the regulatory sequences of the invention. For example, transcriptional enhancers and/or sequences which allow inducible expression of the regulatory sequences of the invention may be employed. An example of a suitable inducible system is tetracycline-regulated gene expression. The regulatory sequence of the invention may be derived from the GREP genes of Oryza sativa or of Arabidopsis thaliana, although other plants may be suitable sources for such regulatory sequences as well.

Usually, said regulatory sequence is part of a recombinant DNA molecule. In a preferred embodiment of the present invention, the regulatory sequence in the recombinant DNA molecule is operatively linked to a heterologous DNA sequence.

In a preferred embodiment, the heterologous DNA sequence of the above-described recombinant DNA molecules encodes a peptide, protein, antisense RNA, sense RNA and/or ribozyme. The recombinant DNA molecule of the invention can be used alone or as part of a vector to express heterologous DNA sequences, which, e.g., encode proteins for, e.g., the control of disease resistance, modulation of nutrition value or diagnostics of GREP related gene expression. The recombinant DNA molecule or vector containing the DNA sequence encoding a protein of interest is introduced into the cells which in turn produce the protein of interest. For example, the regulatory sequences of the invention can be operatively linked to sequences encoding Barstar and Barnase, respectively, for use in the production of male and female sterility in plants.

GREP regulatory sequences may also be used to drive expression of scorable marker, e.g., luciferase, green fluorescent protein or β-galactosidase. This embodiment is particularly useful for simple and rapid screening methods for compounds and substances described hereinbelow capable of modulating GREP specific gene expression. For example, a cell suspension can be cultured in the presence and absence of a candidate compound in order to determine whether the compound affects the expression of genes which are under the control of regulatory sequences of the invention, which can be measured, e.g., by monitoring the expression of the above-mentioned marker. It is also immediately evident to those skilled in the art that other marker genes may be employed as well, encoding, for example, a selectable marker which provides for the direct selection of compounds which induce or inhibit the expression of said marker.

The regulatory sequences of the invention may also be used in methods of antisense approaches. The antisense RNA may be a short (generally at least 10, preferably at least 14 nucleotides, and optionally up to 100 or more nucleotides) nucleotide sequence formulated to be complementary to a portion of a specific mRNA sequence and/or DNA sequence of the gene of interest. Standard methods relating to antisense technology have been described; see, e.g., Klann (1996). Following transcription of the DNA sequence into antisense RNA, the antisense RNA binds to its target sequence within a cell, thereby inhibiting translation of the mRNA and down-regulating expression of the protein encoded by the mRNA. Thus, in a further embodiment, the invention relates to nucleic acid molecules of at least 15 nucleotides in length hybridizing specifically with a regulatory sequence as described above or with a complementary strand thereof.

The present invention also relates to vectors, particularly plasmids, cosmids, viruses and bacteriophages, used conventionally in genetic engineering, that comprise a recombinant DNA molecule of the invention. Preferably, said vector is an expression vector and/or a vector further comprising a selection marker for plants. Methods which are well known to those skilled in the art can be used to construct recombinant vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1989). Alternatively, the recombinant DNA molecules and vectors of the invention can be reconstituted into liposomes for delivery to target cells.

The present invention furthermore relates to host cells transformed with a regulatory sequence, a DNA molecule or vector of the invention. Said host cell may be a prokaryotic or eukaryotic cell.

In a further preferred embodiment, the present invention provides a method for the production of transgenic plants, plant cells or plant tissue comprising the introduction of a nucleic acid molecule, recombinant DNA molecule or vector of the invention into the genome of said plant, plant cell or plant tissue. For the expression in plant cells of a heterologous DNA sequence under the control of a GREP regulatory sequence, further regulatory sequences such as poly(A) tail may be fused, preferably 3′ to the heterologous DNA sequence. Matrix Attachment Sites may be added at the borders of the transgene to act as “delimiters” and insulate against methylation spread from nearby heterochromatic sequences.

Thus, the present invention relates also to transgenic plant cells which contain stably integrated into the genome a recombinant DNA molecule or vector according to the invention. Furthermore, the present invention also relates to transgenic plants and plant tissue comprising the above-described transgenic plant cells. These plants may show, for example, altered growth characteristics. In yet another aspect the invention also relates to harvestable parts and to propagation material of the transgenic plants according to the invention which contain transgenic plant cells described above. Harvestable parts and propagation material can be in principle any useful part of a plant.

Plant cell growth rate and/or the inhibition of plant cell growth can be influenced by (partial) elimination of a gene or reducing the expression of a gene encoding a GREP. Said plant cell growth rate and/or the inhibition of a plant cell growth can also be influenced by eliminating or inhibiting the activity of subject GREP by using for instance, antibodies directed against said protein. As a result of said elimination or reduction, smaller plants or specific organs or tissues can be obtained. Plants or specific organs or tissues which are smaller in volume and in mass may also be obtained.

The growth rate of a plant cell can also be influenced in a transformed plant by overexpression of a sequence according to the invention. Said transformed plant can be obtained by transforming a plant cell with a gene encoding a subject GREP or fragment thereof alone or in combination. The plant cell may belong to a monocotyledonous or dicotyledonous plant. For this purpose, preferentially tissue specific promoters may be used. Therefore, an important aspect of the invention is a method to modify plant architecture by overproduction or reduction of expression of a sequence according to the invention under the control of a tissue, cell or organ specific promoter.

Another aspect of the present invention is a method to modify the growth pattern of plants or of specific organs of a plant caused by environmental stress conditions by appropriate use of sequences according to the invention.

In another aspect of the invention, one or more subject DNA sequences, vectors or proteins, regulatory sequences or recombinant DNA molecules of the invention or the antibody hereinbefore described, or compound, may be used to modulate, for instance, cell growth rates of storage cells, storage tissues and/or storage organs of plants or parts thereof.

Preferred target storage organs and parts thereof for the modulation of cell growth are, for instance, seeds (such as from cereals, oilseed crops), roots (such as in sugar beet), tubers (such as in potato) and fruits (such as in vegetables and fruit species). Furthermore it is expected that increased cell growth in storage organs and parts thereof correlates with enhanced storage capacity and as such with improved yield. In yet another embodiment of the invention, a plant with modulated cell growth in the whole plant or parts thereof can be obtained from a single plant cell by transforming the cell, in a manner known to the skilled person, with the above-described means.

In view of the foregoing, the present invention also relates to the use of a DNA sequence, vector, protein, antibody, regulatory sequences, recombinant DNA molecule, nucleic acid molecules or compound of the invention for modulating plant cell growth, for influencing the activity of GREPs, for disrupting plant cell growth by influencing the presence or absence or by interfering in the expression of a GREP, for modifying growth inhibition of plants caused by environmental stress conditions, for inducing male or female sterility, for influencing cell growth in a host as defined above or for use in a screening method for the identification of receptors or other trans acting factors of GREPs.

In addition the use of the subject nucleic acid molecules for the genetic engineering of plants with modified growth characteristics and/or their use to identify homologous molecules, the subject nucleic acid molecules may also be used for several other applications, for example, for the identification of nucleic acid molecules which encode proteins which interact with the GREP proteins described above. This can be achieved by assays well known in the art such as the so-called yeast ‘two-hybrid system (see Example 8). In this system, the protein encoded by a subject nucleic acid molecule or a part thereof is linked to the DNA-binding domain of transcription factor such as GAL4. A yeast strain expressing this fusion protein and comprising a lacZ reporter gene driven by an appropriate promoter, which is recognized by the GAL4 transcription factor, is transformed with a library of cDNAs which will express plant proteins or peptides thereof fused to a transcription activation domain. Thus, if a peptide encoded by one of the cDNAs is able to interact with the fusion peptide comprising a peptide or a protein of the invention, the complex is able to direct expression of the reporter gene. In this way the nucleic acid molecules according to the invention and the encoded peptide can be used to identify peptides and proteins interacting with GREPs. It is apparent to the person skilled in the art that this and similar systems may then further be exploited for the identification of inhibitors of the binding of the interacting proteins.

Other methods for identifying compounds which interact with the proteins according to the invention or nucleic acid molecules encoding such molecules are, for example, the in vitro screening with the phage display system as well as filter binding assays or ‘real time’ measuring of interaction using, for, example, the BIAcore apparatus (Pharmacia); see references cited supra.

Some other applications for the use of the genes and proteins of the present invention are illustrated below. These applications are also useful for the OsPSK growth regulating protein, which is closely related to the growth regulating proteins of the present invention, but which do not contain the GREP motif.

Co-expression of PSK or GREP and its receptor(s). Important cell plant-cell communication processes occur via the binding of a ligand to its receptor(s). These communications make use of small compounds such as auxin, cytokinin, gibberellin etc., but also these communications can make use of peptides. Therefore it is likely that as suggested for PSK, the GREP also mediates its effects on the host cell via the binding of its receptor. Typically, receptors are located at the outer surface of the plant plasma membrane, they have transmembrane domain and a intracellular domain to mediate signal transduction. Typically also two types of receptor binding affinities are described: a low affinity for basal expression and a high affinity for rapid response to growth conditions. Different receptors for the PSK peptide have already been described (Matsubayashi et al., 2000) wich have different binding affinity and different state of activity.

Therefore a particular embodiment of the present invention is a method for altering growth and/or development in a plant or plant cell comprising expression in said plant of a nucleic acid encoding a GREP or OsPSK growth regulating protein in combination with modulating the functionality of the receptor for said GREP or OsPSK growth regulating protein.

Identification of the putative receptor for the GREP or PSK peptides can be achieved via methods well known by the person skilled in the art.

For example a method comprising the steps of radioactive labelling of the PSK, mixing with plant extract, UV cross-linking or other cross-linking of the proteins, 2D gel electrophoresis, mass spectrometry on the radioactive spot to identify the amino acid composition for the receptor. Another method is to generate antibodies against PSK, that can subsequently be used for pull-down experiments, followed by mass spectrometry on the pulled down protein fraction. A third method to identify the PSK or the GREP receptor, is to perform a Two-Hybrid screen (Clontech) with the PSK or GREP genes or parts thereof as a bait. This screen will be performed on a cDNA subset consisting of genes or parts thereof encoding transmembrane domains as predicted in the database. For example, such a subset was predicted on the Arabidopsis genome and this category of predictions is available publicly (such as in from NCBI or MIPS). This subset contains approximately 700 nucleic acids. Again smaller subsets can be used for this screen, e.g. the PSK receptor presumably belongs to class of LRR receptor-like kinases and this subset contains approximately 200 candidates. In analogy to PSK, the GREP receptor can also belong to this class of proteins.

Another application of the present invention is to express the PSK or GREP encoding genes while at the same time the receptor is modified to be constitutively “on”. For example this is achieved by influencing the activity of the kinases that regulate the functionality of the receptor. For example, blocking the amino acid of the receptor that has to be phosphorylated or dephosphorylated or blocking the activity of the stimulatory or activating kinases or phosphatases that are involved in the functionality of the receptor. This means that the receptor does not necessarily need to be co-expressed but the functionality of the receptor is influenced in combination with the expression of the PSK or GREP transgene expression. This can be achieved as described above.

Accordingly, a particular embodiment of the present invention is a method for altering growth and/or development in a plant or plant cell comprising co-expression in said plant of a first nucleic acid encoding a GREP or an OsPSK growth regulating protein and a second nucleic acid encoding a protein that is involved in the post-translational processing or the biological functionality of said GREP or OsPSK growth regulating protein.

In another application of the present invention, it is the purpose to ensure that the produced GRP or PSK in the host cell is biologically active. This means that if the level of GREP or PSK in the host cell is altered, particularly increased, by using the genes and/or the proteins of the present invention, these proteins must also be biologically active. Taking into account that post-translational modification processes undergone by the GREP's or PSK's might be essential for this biological activity of PSK or GREP proteins, it is important that also these post-translational modifications processes can take place sufficiently.

Accordingly, in a particular application of the present invention the cDNA's as described above are ectopically expressed in a host cell in combination with a second transgene encoding protein that is involved in the post-translational processing of the PSK or the GREP protein.

Therefore a particular embodiment of the present invention is a method for altering growth and/or development in a plant or plant cell comprising co-expression in said plant of a first nucleic acid encoding a GREP or an OsPSK growth regulating protein and a second nucleic acid encoding a protein that is involved in the post-translational processing or the biological functionality of said GREP or OsPSK growth regulating protein. One example of such an approach is described below.

Co-expression of tyrosylprotein sulphotransferase with any PSK may be preferred. Tyrosine sulfation is a late post-transcriptional modification usually affecting membrane or secreted proteins, and this sulfation is important for protein-protein interaction. Possibly this sulfation process also modulates the PSK-alpha activity. In vitro experiments showed that a synthetic PSK is inactive when it is not sulfated (Matsubayashi et al., 1996). The two tyrosine residues in the PSK sequence are in an acidic amino acid context which suggests that both tyrosine residues of the conserved motif may undergo sulfation (Yang et al., 2000). Also In the GREP motif the two tyrosines are in a similar context and therefore the GREPS may also undergo the post-translational sulfation.

Over-expression of PSK or GREP encoding genes alone may not lead to active growth signaling if the activity of post-translational modification enzymes are limiting. Co-expression of both the signaling peptide itself (PSK or GREP) and the post-translational modification enzymes, such as sulfation enzymes may lead to enhanced biological activity of the ligand, and thus increased proliferation of the host cells. Therefor proteins involved in sulfation, more particularly in tyrosin sulfation processes are preferred candidates for co-expression.

Therefor in a particular application of the present invention the cDNA's as described above are ectopically expressed in a host cell in combination with a second transgene encoding the protein that is involved in sulfation, such as tyrosine sulfation.

Accordingly, a particular embodiment of the present invention is a method for altering growth and/or development in a plant or plant cell comprising co-expression in said plant of a first nucleic acid encoding a GREP or OsPSK growth regulating protein and a second nucleic acid encoding a protein that is involved in sulphation of said GREP or OsPSK growth regulating protein.

In Matsubayashi et al. (2001), it has been suggested that tyrosine protein sulfotransferase (TPST) could be involved in Y sulfation of PSK's. The enzyme tyrosinylprotein sulfotransferase (TPST) catalyses in higher eukaryotes the transfer of sulfate from phosphoadenosine phosphosulfate (PAPS) to tyrosines within highly acidic motifs of polypeptides. Current evidence in mammalian systems indicates that the enzyme is a membrane-associated protein with a lumenally oriented active site localized in the trans-Golgi network.

Accordingly in a related application of the present invention the cDNA's as described above are ectopically expressed in a host cell in combination with a second transgene encoding a tyrosine protein sulfotransferase

Accordingly, a particular embodiment of the present invention is a method for altering growth and/or development in a plant or plant cell comprising co-expression in said plant of a first nucleic acid encoding a GREP or OsPSK growth regulating protein and a second nucleic acid encoding a tyrosine protein sulphotransferase.

Alternatively, this method of the present invention comprises the expression of GREP or OsPSK in combination with the modulation of the functional activity of tyrosine protein sulfotransferase. This can be achieved by modulating the activity of the endogenous tyrosine protein sulfotransferase, or by administration of tyrosine protein sulfotransferase.

Alternatively another application of the present invention has the purpose to modify the PSK protein or the GREP protein product to be constitutively “on” or to be constitutively active. This does not necessarily mean expression of a first nucleotide encoding a GREP or PSK, but can be achieved for example by modulating the activity of proteins involved in post-translational modifications of the GREP or PSK, such as sulfation proteins, such as tyrosine protein sulfotransferase.

Accordingly, a particular embodiment of the present invention is a method for altering growth and/or development in a plant or plant cell comprising modulation of the activity of a GREP or an OsPSK growth regulating protein by modulating the activity of proteins involved in post-translational modifications or biological activity of said GREP or PSK growth regulating protein, such as sulphation proteins, such as tyrosine protein sulphotransferase.

In a further particular embodiment of the present invention, the nucleotide sequence of said first nucleotide in the methods above is set forth in any of SEQ ID NOs 1, 3, 5, 7, 8, 10, 11, 13, 14, 16, 18, 19, 22, 24, 25, 27, 28, 30, 32, 34, 36, 38, 40, 42, 44, 46, 48, 50, 53, 54, 56, 58, 60, 62, 64, 66, 68, 69, 71, 72, 74, 76, 78, 80, 82, 84, 86, 88, 90, 92, 94, 96, 98, 100, 102 or 104.

Preferable the combined transgenes in these co-expression applications originate from the same plant species and are co-expressed in that plant of origin. For example, a rice GREP gene is combined with a GREP receptor encoding gene and are both transformed in a rice cell.

Alternatively, GREP genes and receptor genes of different plant species can be combined and transformed into said the same or different plant species.

With respect to the fact that the inventors identified a large amount of GREP family members in the same plant (e.g. 7 family members of Arabidopsis so far), these family members could behave slightly different in the plant (i.e. have slightly different functionality additional to their basic function of signalling peptide and/or growth regulator). Also it is possible that they differ in functionality according to their place and time of expression.

Therefore, for each of these family members there could be one or more receptors available or one or more post-translational modification proteins so that each family member can exert its specific functionality. The receptor and other interacting proteins for PSK's and GREP's or the different receptors and binding proteins for the different PSK's and GREP's can be identified by e.g. co-immunoprecipitation experiments, cross-linking or two-hybrid as described above.

Also the different PSK genes and GREP genes can be tissue specific or active only in a particular stage of development, or during particular environmental conditions.

Accordingly, in a particular embodiment of the present invention, a GREP encoding gene or PSK encoding gene is combined with a gene which influences the GREP or PSK activity, and which is active in the same tissue and/or in the same stage of development and/or during the same environmental conditions.

Furthermore, it is possible to use the nucleic acid molecules according to the invention as molecular markers in plant breeding. Moreover, the overexpression of nucleic acid molecules according to the invention may be useful for the alteration or modification of plant/pathogen interaction. The term “pathogen” includes, for example, bacteria, viruses and fungi as well as protozoa.

In accordance with the present invention, growth characteristics of plants may be modified by introducing into a plant or plant cell, a GREP. For example, a GREP may be introduced into the plant cell by micro-injection, permeation, or biolistics. Alternatively, growth characteristics of a plant or plant cell are achieved by introducing into a plant cell a nucleic acid molecule encoding a GREP under the control of a promoter and/or other regulatory sequences which function in plants. Plants with altered growth characteristics are obtained by regenerating the transformed plant cell into a plant. Methods of introducing nucleic acid molecules into plant cells are well known in the art and discussed herein. Usually, the nucleic acid molecule encoding a GREP under the control of a regulatory region is in the form of a vector or genetic construct as hereinbefore described. The genetic construct when expressed in a cell is able to alter the signal transduction pathway controlled by a GREP. Preferentially, such genetic construct consists of a GREP protein expressed under control of a regulated promoter.

The methods of the present invention include, e.g., altering growth rates or biomass, size or number of plant cells, or of specific organs or tissues of a plant such as roots, leaves, flowers, seeds, stems, etc. Different cell types may be targeted such as e.g., epidermal cells, meristematic cells, palissade cells, mesophyl cells, etc. Preferably, plant cell size and biomass is increased and growth rates enhanced but they may also be reduced or downregulated. The resultant transgenic plants which express a GREP of the present invention are also provided. For example, in order to disrupt plant cell growth in a certain organ or tissue, a GREP activity is downregulated. A method for increasing the level of GREP activity is also provided. This method comprises introducing into a plant cell a GREP under the control of a regulatory sequence which controls the expression of the GREP.

The aforementioned methods result in plant cells and plant parts and/or whole plants exhibiting altered characteristics. For example, the present invention provides a transgenic plant, an essentially derived variety thereof, a plant part, or plant cell which comprises a nucleotide sequence encoding a GREP under the control of a promoter which functions in plants wherein said nucleotide sequence encoding a GREP is heterologous to the genome of the transgenic plant or has been introduced into the transgenic plant, plant part or plant cell by recombinant DNA means.

In another preferred embodiment, GREPs may be expressed under control of a seed-specific promoter in cereals, such as wheat, barley, rice and maize. Changes in seed growth can alter the size, and possibly protein and starch composition of the seed, thereby increasing yields and altering its storage capacity and processing properties (e.g. for brewery and bread-making industry). Other modifications in seed size and composition can be obtained by expressing GREPs under control of promoters that are specific for a specific seed tissue (e.g. embryo- or endosperm-specific) or developmental stage.

In another preferred embodiment, GREPs may be expressed under control of a root- or tuber specific promoter in root and tuber crops such as turnips, sugarbeet, radish, carrot, potato, yams and cassava in order to alter cell size, shape, number, storage capacity and yield.

In yet another embodiment, GREPs may be expressed under the control of leaf-specific promoters or tissue-specific promoters (e.g. epidermis specific, L2 layer specific) with the aim of increasing leaf size in ornamental plants and in vegetables of which the leaves are consumed (e.g. lettuce, cabbage, endive).

In still another embodiment of the invention, the increased leaf size may also improve the ability of the plant in capturing light, thereby increasing its photosynthesis capacity and crop productivity.

Preferred promoters may contain additional copies of one or more specific regulatory elements, to further enhance expression and/or to alter the spatial expression and/or temporal expression of a nucleic acid molecule to which it is operably connected. For example, copper-responsive, glucocorticoid-responsive or dexamethasone-responsive regulatory elements may be placed adjacent to a heterologous promoter sequence driving expression of a nucleic acid molecule to confer copper inducible, glucocorticoid-inducible, or dexamethasone-inducible expression respectively, on said nucleic acid molecule. Examples of promoters that may be used in the performance of the invention are provided in Tables 1 and 2. The promoters listed in these tables are provided for the purposes of exemplification only and the present invention is not to be limited by the list provided therein. Those skilled in the art will readily be in a position to provide additional promoters that are useful in performing the present invention. The promoters listed may also be modified to provide specificity of expression as required. In each of the preceding embodiments of the present invention, a GREP or a homologue, analogue, or derivative thereof, is expressed under the operable control of a plant-expressible promoter sequence. As will be known to those skilled in the art, this is generally achieved by introducing a genetic construct or vector into plant cells by transformation or transfection means. The nucleic acid molecule or a genetic construct comprising it may be introduced into a cell using any known method for the transfection or transformation of said cell. Wherein a cell is transformed by the genetic construct of the invention, a whole organism may be regenerated from a single transformed cell, using methods known to those skilled in the art.

A whole plant may be regenerated from the transformed or transfected cell, in accordance with procedures well known in the art. Plant tissue capable of subsequent clonal propagation, whether by organogenesis or embryogenesis, may be transformed with a genetic construct of the present invention and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem).

The generated transformed plants may be propagated by a variety of means, such as by clonal propagation or classical breeding techniques. For example, a first generation (or T1) transformed plant may be selfed to give homozygous second generation (or T2) transformants, and the T2 plants further propagated through classical breeding techniques.

The generated transformed organisms contemplated herein may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

A further aspect of the present invention clearly provides the genetic constructs and vectors designed to facilitate the introduction and/or expression and/or maintenance of the GREP-encoding sequence and promoter into a plant cell, tissue or organ.

In addition to the GREP-encoding sequence and promoter sequence, the genetic construct of the present invention may further comprise one or more terminator sequences. Those skilled in the art will be aware of promoter and terminator sequences which may be suitable for use in performing the invention. Such sequences may readily be used without any undue experimentation.

The genetic constructs of the invention may further include an origin of replication sequence which is required for maintenance and/or replication in a specific cell type, for example a bacterial cell, when said genetic construct is required to be maintained as an episomal genetic element (e.g. plasmid or cosmid molecule) in said cell. Preferred origins of replication include, but are not limited to, the f1-ori and colE1 origins of replication. The genetic construct may further comprise a selectable marker gene or genes that are functional in a cell into which said genetic construct is introduced. As used herein, the term “selectable marker gene” includes any gene which confers a phenotype on a cell in which it is expressed to facilitate the identification and/or selection of cells which are transfected or transformed with a genetic construct of the invention or a derivative thereof. Suitable selectable marker genes contemplated herein include the ampicillin resistance (Amp^{r}), tetracycline resistance gene (Tc^{r}), bacterial kanamycin resistance gene (Kan^{r}), phosphinothricin resistance gene, neomycin phosphotransferase gene (nptII), hygromycin resistance gene, β-glucuronidase (GUS) gene, chloramphenicol acetyltransferase (CAT) gene, green fluorescent protein (gfp) gene (Haseloff et al., 1997), and luciferase gene, amongst others.

The present invention is applicable to any plant, in particular monocotyledonous plants and dicotyledonous plants including a fodder or forage legume, companion plant, food crop, tree, shrub, or ornamental. Examples of plants which can serve as sources of the subject GREP nucleic acid molecules or peptides or which may be transformed with the subject isolated nucleic acid molecules include but are not limited to: Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chaenomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Diheteropogon amplectens, Dioclea spp, Dolichos spp., Dorycnium rectum, Echinochloa pyramidalis, Ehrartia spp., Eleusine coracana, Eragrestis spp., Erythrina spp., Eucalyptus spp., Euclea schimperi, Eulalia villosa, Fagopyrum spp., Feijoa sellowiana, Fragaria spp., Flemingia spp, Freycinetia banksii, Geranium thunbergii, Ginkgo biloba, Glycine javanica, Gliricidia spp, Gossypium hirsutum, Grevillea spp., Guibourtia coleosperma, Hedysarum spp., Hemarthia altissima, Heteropogon contortus, Hordeum vulgare, Hyparrhenia rufa, Hypericum erectum, Hyperthelia dissoluta, Indigo incamata, Iris spp., Leptarrhena pyrolifolia, Lespediza spp., Lettuca spp., Leucaena leucocephala, Loudetia simplex, Lotonus bainesii, Lotus spp., Macrotyloma axillare, Malus spp., Manihot esculenta, Medicago sativa, Metasequoia glyptostroboides, Musa sapientum, Nicotianum spp., Onobrychis spp., Omithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativum, Podocarpus totara, Pogonarthria fleckii, Pogonarthria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys verticillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda tnandra, Trifolium spp., Triticum spp., Tsuga heterophylla, Vaccinium spp., Vicia spp. Vitis vinifera, Watsonia pyramidata, Zantedeschia aethiopica, Zea mays, rice, straw, amaranth, onion, asparagus, sugar cane, soybean, sugarbeet, sunflower, carrot, celery, cabbage, canola, tomato, potato, lentil, flax, broccoli, oilseed rape, cauliflower, brussel sprout, artichoke, okra, squash, kale, collard greens, and tea, amongst others, or the seeds of any plant specifically named above or a tissue, cell or organ culture of any of the above species.

This aspect of the invention further extends to plant cells, tissues, organs and plants parts, propagules and progeny plants of the primary transformed or transfected cells, tissues, organs or whole plants that also comprise the introduced isolated nucleic acid molecule operably under control of the cell-specific, tissue-specific or organ-specific promoter sequence and, as a consequence, exhibit similar phenotypes to the primary transformants/transfectants or at least are useful for the purpose of replicating or reproducing said primary transformants/transfectants.

‘Downregulation of expression’ as used herein means lowering levels of gene expression and/or levels of active gene product and/or levels of gene product activity (see Example 10). Decreases in expression may be accomplished by e.g. the addition of coding sequences or parts thereof in a sense orientation (if resulting in co-suppression) or in an antisense orientation relative to a promoter sequence and furthermore by e.g. insertion mutagenesis (e.g. T-DNA insertion or transposon insertion) or by gene silencing strategies as described by e.g. Angell and Baulcombe, 1998—WO9836083, Lowe et al., 1989—WO9836083), Lederer et al., 1999—WO9915682 or Wang et al., 1999—WO9953050. Genetic constructs aimed at silencing gene expression may have the nucleotide sequence of said gene (or one or more parts thereof) positioned in a sense and/or antisense orientation relative to the promoter sequence. Another method to downregulate gene expression comprises the use of ribozymes, e.g. as described in Atkins et al., 1994—WO9400012, Lenee et al., 1995—WO9503404, Lutziger et al, 2000—WO0000619, Prinsen et al, 1997—WO9713865 and Scott et al., 1997—WO9738116.

Modulating, including lowering, the level of active gene products or of gene product activity can be achieved by administering or exposing cells, tissues, organs or organisms to said gene product, a homologue, analogue, derivative and/or immunologically active fragment thereof. Immunomodulation is another example of a technique capable of down-regulating levels of active GREP gene product and/or gene product activity and comprises administration of or exposing to or expressing antibodies to said GREP gene product to or in cells, tissues, organs or organisms wherein levels of said gene product and/or gene product activity are to be modulated. Such antibodies comprise “plantibodies”, single chain antibodies, IgG antibodies and heavy chain camel antibodies as well as fragments thereof.

A particularly preferred embodiment of the present invention is a method to regulate the growth of a plant or an organ or tissue or cell of a plant by contacting said plant cell, organ or tissue with a GREP protein or preferably with the active product derived from a GREP protein. Since GREPs or the active product derived from GREPs are growth regulators, they can be used as additives in plant cell growth media for in vitro cultures. Alternatively, they can be applied directly to the plant or plant part as part of a formulation in a liquid or solid composition.

Another embodiment of the present invention is a method to introduce specific GREP alleles from a donor to a recipient elite plant genome by marker-assisted selection in plant breeding programs. The effects of specific GREP alleles on phenotype are determined to identify desirable GREP alleles. Molecular markers that are linked to these GREP alleles are developed. As disclosed herein, GREP genes can be isolated from any plant species and the GREP sequence polymorphisms can be used for the development of such molecular markers. In addition, several other techniques exist to identify molecular markers linked to a trait of interest that are known to a person skilled in the art.

Another embodiment of the invention relates to a method for identifying regulatory sequences of GREP growth regulating polypeptide-genes comprising:

(a) hybridizing a nucleic acid encoding a GREP growth regulating polypeptide, against a plant genomic library,

(b) isolating the genomic sequence corresponding to said GREP growth regulating polypeptide,

(c) cloning the 5′ upstream genomic fragment of said GREP growth regulating polypeptide-gene in front of a marker gene,

(d) introducing the resulting chimeric gene into a plant or plant cell for transient exression, and

(e) inferring from the expression pattern the presence of a regulatory sequence in said chimeric construct.

The invention also relates to an isolated nucleic acid molecule encoding a protein having an amino acid sequence as set forth in SEQ ID NO 2 or a nucleic acid comprising a nucleotide sequence as set forth in SEQ ID NO 1.

The invention also relates to an isolated nucleic acid molecule encoding a protein having an amino acid sequence as set forth in SEQ ID NO 12 or a nucleic acid comprising a sequence as set forth in SEQ ID NO 10 or SEQ ID NO 11.

The invention also relates to an isolated nucleic acid molecule encoding a protein having an amino acid sequence as set forth in SEQ ID NO 70 or a nucleic acid comprising a nucleotide sequence as set forth in SEQ ID NO 69 or SEQ ID NO 68.

The invention also relates to an isolated nucleic acid molecule encoding a protein having an amino acid sequence as set forth in SEQ ID NO 73 or a nucleic acid comprising a nucleotide sequence as set forth in SEQ ID NO 72 or SEQ ID NO 71.

The following examples further illustrate the invention.

### EXAMPLES

### Example 1

### Plant Material and Incubation Conditions

Unless stated otherwise in the examples, all recombinant DNA techniques are performed according to protocols as described in Sambrook et al. (1989), Molecular Cloning: A Laboratory Manual. Cold Spring Harbor Laboratory Press, NY or in Volumes 1 and 2 of Ausubel et al. (1984), Current Protocols in Molecular Biology, Current Protocols. Standard materials and methods for plant molecular work are described in Plant Molecular Biology Labfase (1993) by R. D. D. Croy, jointly published by BIOS Scientific Publications Ltd (UK) and Blackwell Scientific Publications (UK).

Seeds of deepwater rice (Oryza sativa L., cultivar Pin Gaew 56) were obtained from the International Rice Research Institute (Los Banos, Philippines). Rice plants were grown for 12 to 14 weeks as described (Sauter, 1997). All experiments were carried out under continuous light (200 μE m⁻² s⁻¹) at 25° C. in a growth chamber. For growth induction, whole plants were submerged in a 600-L plastic tank filled with tap water at 25° C. with approximately 30 cm of the leaf tips remaining above the water surface as described (Lorbiecke & Sauter, 1998). Control plants were kept in the same growth chamber.

Analysis of hormone and inhibitor effects was performed using excised stem segments containing the youngest growth-responsive internode (Raskin & Kende, 1984). Growth of the stem sections was induced by application of 50 μM GA₃ for the times indicated. To inhibit protein synthesis, stem sections were incubated in aqueous solutions of cycloheximide for the times indicated. Plant tissue for RNA extraction was harvested on ice and immediately after harvest frozen in liquid nitrogen. Meristematic tissue was harvested from 0 to 5 mm above the second youngest node, i.e. the intercalary meristem (IM). Cells which are predominately involved in elongation were harvested from 5 to 15 mm above the second youngest node, i.e. the elongation zone (EZ) and differentiated tissue was harvested from the oldest part of the internode below the youngest node, i.e. the differentiation zone (DZ).

To analyse gene expression in seedlings, 40 seeds were germinated for seven days on moist filter paper in darkness using a black pot covered with aluminum foil or in a light/dark cycle in a mini-greenhouse. All seeds were kept in a growth chamber at the conditions described above. The seedlings that were germinated in continuous darkness were harvested under green light in a darkroom. Indica rice cultivar IR43 suspension-cultured cells were obtained from Drs. G. Biswas and I. Potrykus (Institute of Plant Sciences, Swiss Federal Institute of Technology, ETH, Zürich, Switzerland; Biswas et al., 1994) and subcultured weekly in MS Medium (Murashige & Skoog, 1962) supplemented with 1 mg I⁻¹ 2,4-D. Cells were harvested 5 days after subculturing, at which time they were in logarithmic growth phase. Cells were immediately frozen in liquid nitrogen and used for RNA isolation as described (Lorbiecke & Sauter, 1998).

### Example 2

### Molecular Cloning and Sequence Analyis of the Submergence-Induced Gene OsGREP1 in Deepwater Rice

For the identification of submergence-induced genes in adventitious roots of deepwater rice, a PCR-based subtractive hybridization procedure was performed according to the method described by (Buchanan-Wollaston & Ainsworth, 1997). As driver population, cDNA was used derived from mRNA of adventitious root primordia located at the third node of unsubmerged deepwater rice plants. The target cDNA populations were generated from adventitious roots of plants partially submerged for 2 h. Both cDNA populations were digested into smaller fragments using the restrictrion enzymes AIul and Rsal. Driver- or target-specific adapters were ligated to the cDNA fragments and driver cDNA was amplified with biotinylated primers corresponding to the adapter sequence. Target cDNA was amplified with target adapter-specific primers. Target cDNA was mixed with excess driver cDNA and hybridized at 65° C. for 20 h. The biotinylated fragments and their hybridizing complements were removed using streptavidin-coated paramagnetic beads (Dynal, Oslo, Norway). The remaining cDNA was hybridized for 2 h with excess driver cDNA. Following magnetic separation, non-hybridizing target cDNA was amplified by PCR using target adaptor-specific primers. After two additional rounds of long and short hybridizations combined with magnetic separation and PCR amplification as described above, the resulting enriched cDNAs were cloned into pBluescript. Clones were used as probes for expression analysis as described (Buchanan-Wollaston & Ainsworth, 1997). Clone SH27 showed higher transcript levels in adventitious roots than in the internode and was furthermore transiently induced in adventitious roots after 2 h submergence. Together, this data indicates that the SH27 transcript plays a role in the submergence-induced root growth process. The SH27 cDNAs was sequenced from both sides by the dideoxynucleotide chain termination method (Sanger et al., 1977) with the ABI PRISM Dye Terminator Sequencing Kit (Applied Biosystems, Weiterstadt, Germany). Clone SH27 contains a cDNA of 304 bp encoding a partial open reading frame of 38 amino acids followed by a TGA stopcondon and 185 bp of the 3′untranslated region (3′UTR). The 5′end of the SH27 cDNA was directly amplified from a λZAPII cDNA library of deepwater rice by PCR using the SH27-specific primer TGGATGGATGGATCGATCGA and the primer GTACCGGGCCCCCCTCGAG, specific for the pBleuscript cDNA vector. The resulting PCR fragment was sequenced as described above and contained a complete ORF encoding a putative protein of 119 amino acids. A homology search in a protein database using this peptide sequence as query did not reveal any significant homologies. Search of EST and genomic databases with the nucleotide sequence as query turned up several ESTs and genomic clones with significant homology to this sequence. Two rice ESTs (accession number C73667 and D41594) covered non-overlapping parts of the SH27 gene with 100% nucleotide sequence identity: EST D41594 from nt 1 to 263 and EST C73667 from nt 335 to 651 with additional sequences. The remainder of the 3′UTR of the cDNA was virtually derived from the EST clone C73667. The resulting full-length cDNA is 795 bp long and was called OsGREP1 for Oryza sativa Growth Regulating Protein 1. The nucleotide sequence of OsGREP1 is set forth in SEQ ID NO 1. The PCR fragment corresponds to nt 1-486 of SEQ ID NO 1; the initial cDNA sequence of SH27 corresponds to nt 348-651 of SEQ ID NO 1 and the EST C73667 corresponds to nt 335-795 of SEQ ID NO 1. OsGREP1 encodes an open reading frame of 357 bp. The predicted polypeptide is 119 amino acids long with a calculated molecular mass of 12.6 kDa. The amino acid sequence deduced from OsGREP1 is set forth in SEQ ID NO 2. Two in-frame stop codons in the 5′untranslated region (5′UTR) at nucleotides 71 to 73 and 86 to 88 indicate that OsGREP1 comprises the complete coding region of the putative protein. The 5′UTR further has CTC and ATC repeats with unknown significance. The complete nucleotide sequence of OsGREP1 with indication of the open reading frame is represented in FIG. 1a.

The nulceotide sequence of OsGREP1 was cloned and confirmed by sequence analysis. In the FIG. 18 (“figure all sequences) an alignment of the two alternative protein sequences is shown. The new sequence SEQ ID NO 54 has 1 nucleotide difference: C at position 92 instead of T. This nulceotide difference results in 1 amino acid substitution: S in SEQ ID NO 55 at position 31 instead of F

A hydropathy blot for the OsGREP1 protein is shown in FIG. 1b. Positive numbers indicate hydrophobic polypeptide regions. The broad bar at the N-terminus indicates a putative signal peptide and an acidic domain is indicated with a thin line. A signal peptide for targeting OsGREP1 to the secretory pathway is predicted by SignalP V1.1 with most likely cleavage site between pos. 34 and 35: AAA-AR (Nielsen et al., 1997) indicated with an arrowhead in FIG. 1a. Secondary structure analysis for the OsGREP1 protein was determined according to Stultz et al. (1993) and is shown in FIG. 1c. The probability for an α-helical structure is given as a line. It is nearly 1 for the signal peptide region and three additional regions in the post-leader sequence. The probability for a turn is indicated by a shaded curve. The highest probability for a turn exists at around position 70 between helix 1 and 2 of the post-leader sequence.

### Example 3

### Genomic Organization of OsGREP1 in Deepwater Rice

The genomic organization of OsGREP1 was tested by DNA gel blot analysis. Genomic DNA was isolated from Oryza sativa L cv. Pin Gaew 56 DNA as described (Dellaporta et al., 1983), digested with four different restriction enzymes that do not cut within the OsGREP1 cDNA sequence and separated by electrophoresis on a 1% (w/v) agarose gel. The DNA was capillary blotted to a nylon membrane (Hybond N+; Amersham, Braunshweigh, Germany) and hybridized with a gene-specific ³²P-labeled probe prepared according to the manufacturer's instructions (Amersham). Hybridization was performed overnight at 68° C. in 1% SDS, 1M NaCl, 10% dextran sulphate and 70 μg/ml fish sperm DNA. The membranes were washed under stringent conditions using 2×SSC; 0.1% SDS, and 15 minutes using 1×SSC; 0.1% SDS at 68° C. Signals were revealed by autoradiography. For 2 out of 4 digests, only one band could be detected, while the other 2 digests revealed two hybridizing bands. Since only a single band can be detected for 2 different digests, the OsGREP1 gene does not have highly related sequences (i.e. over 90% sequence identity) in the deepwater rice genome. The two bands observed for the other 2 digests likely indicate the presence of intron sequences. This is further confirmed by the presence of intron sequences for OsGREP1 homologues in Arabidopsis thaliana since the presence and position of intron sequences is often conserved for gene family members of different plant species.

### Example 4

### Computational Analysis

OsGREP1 homologues were searched using the BLAST 2.0.3 program (Altschul et al., 1997) against the actual releases of public protein and nucleic acid databases available either on a local server or at the NCBI and TIGR website. Identified ESTs representing the same gene were virtually combined to obtain the complete sequence information of the putative open reading frame. The rice EST clones AJ276692 and AJ276693 were obtained from the STAFF institute (Ibaraki, Japan) and sequenced from both sides as described in Example 2. DNA sequence data were analyzed and virtually translated using the Dnasis™ V 5.11 program (Hitachi Software Engineering Co., Ltd. 1984, 1991). Sequences identified as potential homologues of OsGREP1 in the Blast searches were retrieved from the NCBI database and analyzed for specific primary and secondary structure characteristics of the encoded proteins to confirm their relationship with OsGREP1. The secondary structure prediction of proteins was based on discrete state-space modelling using the PSA server (Stultz et al., 1993). For OsGREP1 homologues, similar structural features were obtained using two alternative prediction programs (Kneller et al., 1990; Rost & Sander, 1994). The prediction of hydrophobicity was calculated as described previously (Kyte & Doolittle, 1982). Signal peptide prediction was performed using the SignalP V1.1 WWW Prediction Server (Nielsen et al., 1997). A peptide sequence alignment was calculated for OsGREP1 and the identified homologues and OsPSK with the ClustalX 1.81 program (Thompson et al., 1997) and manually edited using GeneDoc 2.1 (Nicholas, K. B. and Nicholas H. B. Jr. 1997 GeneDoc: Analysis and Visualization of Genetic Variation, http://www.cris.com/˜Ketchup/genedoc.shtml). The phylogenetic tree was displayed using Treeview 1.31 (Page, 1996). A statistics report based on this alignment was calculated with GeneDoc 2.1.

### Example 5

### Identification of OsGREP1 Homologues in Rice and Other Monocot and Dicot Plant Species

Database homology searches were performed to identify EST and/or genomic sequences with homology to the OsGREP1 nucleotide or deduced protein sequence. Since the overall sequence identity at the protein level between different putative homologues can be quite low (see Example 6), secondary structure analysis of the deduced protein was performed to confirm the relationship with OsGREP1. In some cases, confirmed homologues were used as queries in subsequent BLAST searches. Overall, this analyses indicated that the deduced open reading frame of OsGREP1 exhibited significant homology to putative proteins and putative open reading frames of a number of ESTs and genomic sequences of rice, Arabidopsis, soybean, tomato, rape and maize. These homologues were named similar to OsGREP1, with the initials of the genus and species name of the organism from which they were derived, followed by GREP and a number. A complete list is given below.

For rice, two ESTs were obtained from the STAFF institute (Ibaraki, Japan), sequenced and assigned as OsGREP2 for EST AJ276692 and OsGREP3 for AJ276693. OsGREP2 is 820 bp long (SEQ ID NO 3) and bears an ORF of 102 amino acids (SEQ ID NO 4) encoding a protein of 11.0.Kd.

The nulceotide sequence of OsGREP2 was cloned and confirmed by sequence analysis. In FIG. 18, an alignment of the two alternative protein sequences is shown. The new sequence SEQ ID NO 56 has 2 nucleotides difference: A at position 219 instead of G and G at position 243 instead of T. This nulceotide difference results in 1 amino acid substitution: K in SEQ ID NO 57 at position 74 instead of E.

OsGREP3 is 661 bp long (SEQ ID NO 5) and bears an ORF of 83 amino acids (SEQ ID NO 6) encoding a protein of 8.6 Kd. The OsGREP4 cDNA (Acc. MG34212, version AAG34212.1) is 228 bp long (SEQ ID NO 8) and bears an ORF of 75 amino acids (SEQ ID NO 9) encoding a protein of 8.2 Kd.

The database with genomic sequences of Oryza sativa indica from which genomic sequences are publicly available in the form of contigs (http://210.83.138.53/rice/. Beijin Genome Institute). This database was downloaded and saved as a blastable database that was available for the inventors only (on a local server). Publicly there are no protein predictions made for these rice sequences.

This database was blasted with the TBLASTX program (e-value 1000 for little sequence against longer sequences and for similarity allowance) using the peptide sequence consensus for the GREP motif

CX₁X₂X₃CX₄X₅X₆X₇HX₈DYIYTX₉ (SEQ ID NO 52)

wherein X₁ are 4 to 8 amino acids, X₂ is D or E, X₃ is one or two amino acids, X₄ are two or three amino acids, X₅ is R or K, X₆ is R or K, X₇ is any amino acid, X₈ is any amino acid and X₉ is Q or H,

Based on the contigs that were selected via the blast search, the inventor was able to identify the predicted cDNA sequences and the corresponding full-length protein sequence. Based on the homology with the other GREP proteins, the inventor was able to identify the start and stopcodon as well as the intron splicing sites.

Based on the genomic sequence (contig 13167) for OsGREP 5 (SEQ ID NO 68) the cDNA (SEQ ID NO 69) is predicted with the programm Genesplicer (http://www.tigr.org/tigr-scridpts/GeneSplicer/gspl_cgi.cgi). The corresponding amino acid translation is set forth in SEQ ID NO 70. When blasts were performed to the public databases with the genomic sequence, no BAC clones of for example Oryza sativa japonica showed significant homology, but the prediction of the cDNA could be confirmed by the existence of two ESTs (Genbank accession number 25004 and D40931). These two ESTs give a complete overlap with the exons as predicted (so the splicing sites and the intron sequence are correctly predicted), but these ESTs do not contain the GREP motif.

Based on the genomic sequence (contig 3842) for OsGREP 6 (SEQ ID NO 71), the cDNA (SEQ ID NO 72) was predicted with the program Genesplicer. The corresponding amino acid sequence is set forth in SEQ ID NO 73. There are no EST's available in public databases which confirm this splicing prediction of this Oryza sativa indica gene. However, when blasting the prediction against the public database, there is a very high degree of homology found with a BAC clone of Oryza sativa japonica (i.e. BAC clone with Genbank accession number AC103891). For this BAC clone of 124821 nucleotides there were no prediction available. By a comparison with the indica cDNA, the inventors found that there are two nucleotides different between the indica and the japonica sequence. Accordingly the C at postion 205 in the cDNA of indica (SEQ ID NO 72) can be G, and C at position 271 in the indica sequence (SEQ ID NO 72) can be A. These two changes in nucleotide composition also have an effect on the protein translation. Accordingly the amino acids P and P in SEQ ID NO 73 of the indica sequence on postions 69 and 91 could be A and T respectively (in analogy to the japonica sequence translation).

These sequences also comprise the GREP signature motif completely.

For Arabidopsis thaliana, seven OsGREP1 homologues could be identified through searches of public databases, designated AtGREP1 through 7. The AtGREP1 gene was identified and characterized by the inventor. This gene is located on the BAC clone T32N15 (Acc AC002534) identified through homology searches using the BLAST program. Intron/exon prediction in the region 66901 to 69180 of this BAC clone was done by the inventor using the NetPlantGene server (http://www.cbs.dtu.dklservices/NetPGene/), followed by manual inspection of the surrounding sequences, looking for characteristic features of GREP proteins, such as the presence of a signal peptide and the GREP signature motif (see Example 6). The AtGREP1 gene (SEQ ID NO 10) comprises three exons and two introns. The corresponding cDNA (SEQ ID NO 11) is 246 bp long and bears an ORF of 81 amino acids (SEQ ID NO 12) corresponding to a polypeptide with a calculated molecular weight of 9.3 kD. The cDNA and protein sequence of AtGREP1 are not in the public databases and thus represent novel sequences. AtGREP2 corresponds to the predicted gene T20K9.7 (Acc AAC32433.1) located on BAC clone T20K9. The AtGREP2 cDNA is 264 bp long (SEQ ID NO 14) and encodes a putative protein of 87 amino acids long (SEQ ID NO 15) with a calculated molecular weight of 9.6 kD. AtGREP3 is derived from the EST M395590 which was virtually translated. This EST is 445 bp long (SEQ ID NO 16) and encodes a protein of 84 amino acids (SEQ ID NO 17) with a calculated molecular weight of 9.5 kD. EST M720042 is smaller than EST M395590 with 100% sequence identity. AtGREP4 corresponds to the predicted gene F19F18.210 located on the BAC clone ATF19F18. The AtGREP4 cDNA (SEQ ID NO 19) is 264 bp long and encodes a protein of 87 amino acids (SEQ ID NO 20) with a calculated molecular weight of 9.7 kD, annotated as ‘putative protein’ under accession number CAB38311.1.

The nulceotide sequence of AtGREP4 was cloned and confirmed by sequence analysis. In FIG. 18, an alignment of the two alternative cDNA's and an alignment of the two alternative protein sequences are shown. The new cDNA sequence SEQ ID NO 58 is a splice variant of the genomic sequences of SEQ ID NO 18 of AtGREP4 and is an alternative for SEQ ID NO 19. This alternative splicing event results in the new protein sequence as set forth in SEQ ID NO 59.

AtGREP5 corresponds to the predicted gene F21F23.2 located on BAC F21F23. The corresponding cDNA is 204 bp long (SEQ ID NO 22) and encodes a protein of 67 amino acids (SEQ ID NO 23) with a calculated molecular weight of 7.3 kD and which is annotated as ‘contains similarity to a putative protein T16K5.1 . . . ’ under accession number AAF81285.1. AtGREP6 corresponds to the predicted gene T16K5.130 located on BAC ATT16K5. The corresponding cDNA is 213 bp long (SEQ ID NO 25) and encodes a protein of 70 amino acids (SEQ ID NO 26) with a calculated molecular weight of 7.8 Kd. This protein is defined as ‘putative protein’ under accession number CAB66916.1. AtGREP7 corresponds to the predicted gene K14B20.4 located on TAC K14B20. The corresponding cDNA is 234 bp long (SEQ ID NO 28) and encodes a protein of 77 amino acids (SEQ ID NO 29) with a calculated molecular weight of 8.7 Kd. This protein is defined as ‘ . . . similar to unknown . . . ’ under accession number BAB11134.1.

Blast searches using the Arabidopsis thaliana Gene Indices database from TIGR identified a tentative consensus sequence TC93228 which mapped to the same chromosomal region on TAC clone K14B20 as AtGREP7. A tentative consensus sequence is derived from overlapping ESTs and therefore encodes a protein. Amino acid sequence alignments by the applicant demonstrated that the protein encoded by TC93228 and AtGREP7 overlap in their 5′ terminal 41 amino acids but are completely different further downstream. Translation of the genomic sequences showed that the carboxy-terminal amino acids of the TC93228 protein are encoded by a predicted intron sequence. This result indicates that the proteins encoded by TC93228 and AtGREP7 are under control of the same promoter but that alternative splicing gives rise to two transcripts that are identical in their 5′end sequences but then diverge. Alternative splicing thus gives rise to two different proteins and may be a regulatory mechanism for AtGREP7 gene expression.

Overall, the database searches identified 6 different genomic locations containing GREP genes in A. thaliana. The genes AtGREP5, AtGREP2, AtGREP1 and 6, AtGREP4 and AtGREP7 are located on chromosome 1, 2, 3, 4 and 5 of A. thaliana, respectively, indicating that members of the AtGREP gene family are not linked but rather are located on different chromosomes. In view of this data, it can be expected that other plant species will also have rather large gene families as well. This finding is further substantiated by the identification of multiple GREP gene sequences in various other plant species as disclosed herein. Alternative splicing may be used as regulation mechanism for expression of specific GREP genes in these plants as well.

For soybean (Glycine max), six different ESTs were identified, and for two of these a putative ID was assigned in the public database. The GmGREP1 cDNA corresponds to the complementary strand of EST AI856752. This cDNA is 541 bp long (SEQ ID NO 30) and encodes a protein of 93 amino acids (SEQ ID NO 31) which has a calculated molecular weight of 10.4 kD. The GmGREP2 cDNA corresponds to the complementary strand of EST BE658719. This cDNA is 468 bp long (SEQ ID NO 32) and encodes a partial protein of 76 amino acids (SEQ ID NO 33) with an estimated molecular weight of 8.5 kD. The GmGREP3 cDNA corresponds to EST AW185146. This cDNA is 449 bp long (SEQ ID NO 34) and encodes a partial protein of 74 amino acids (SEQ ID NO 35) with an estimated molecular weight of 8.7 kD. Both GmGREP2 and GmGREP3 encode partial proteins, lacking amino-terminal sequences. The GmGREP4 cDNA corresponds to the complementary strand of EST BE820901. This cDNA is 467 bp long (SEQ ID NO 36) and bears an ORF of 79 amino acids (SEQ ID NO 37) encoding a protein of 8.9 Kd. The GmGREP5 cDNA corresponds to the complementary strand of EST BE659360. This cDNA is 398 bp long (SEQ ID NO 38) and bears an ORF of 79 amino acids (SEQ ID NO: 39) with a calculated molecular weight of 8.8 kD. The GmGREP6 cDNA corresponds to EST BE802923 which is 395 bp long (SEQ ID NO 40) and bears an ORF of 79 amino acids (SEQ ID NO 41) with a calculated molecular weight 8.9 kD.

For tomato (Lycopersicon esculentum), the LeGREP1 cDNA (SEQ ID NO 42) is derived from EST AI485184 which is 514 bp long and bears an ORF of 90 amino acids (SEQ ID NO 43) encoding a protein of 10.5 Kd. Other ESTs homologous to LeGREP1 are AI773265 and AI714553. The LeGREP2 cDNA (SEQ ID NO 44) is 490 bp and corresponds to EST AW442998. This cDNA encodes a protein of 83 amino acids (SEQ ID NO 45) with a calculated molecular weight of 9.5 kD.

For rape (Brassica napus), the BnGREP1 cDNA (SEQ ID NO 46) is derived from EST H74648 and is 215 bp long. This cDNA encodes a partial protein of 54 amino acids (SEQ ID NO 47). The BnGREP1 protein lacks amino-terminal sequences and has a calculated molecular weight of 6.1 kD.

For maize (Zea mais), the ZmGREP1 cDNA (SEQ ID NO 48) is 565 bp long and corresponds to the complement of EST AI712273. ZmGREP1 bears an ORF of 98 amino acids (SEQ ID NO 49) encoding a protein of 10.1 Kd. The ZmGREP2 cDNA (SEQ ID NO 50) is 588 bp long and corresponds to the complement of EST AI461518. This cDNA encodes a partial protein of 42 amino acids (SEQ ID NO 51). The ZmGREP2 protein lacks amino-terminal sequences and has a calculated molecular weight of 4.9 kD.

Also new sequences were found on other plant species such as (Ao), Asparagus officinalis; (At), Arabidopsis thaliana; (Bn), Brassica napus; (Ga), Gossypium arboreum; (Gm), Glycine max; (Le), Lycopersicon esculentum; (Mc), Mesembryanthemum cristallinum; (Os), Oryza sativa; (Pt), Pinus taeda; (Sb), Sorghum bicolor, (Sp), Sorghum propinquum; (St), Solanum tuberosum; (Ta), Triticum aestivum; (Zm), Zea mays. (see Table 4, SEQ ID NOs 1 to 103). The corresponding cDNA sequences and protein sequences are shown in FIG. 18.

Also 8 GREP family members in the sugar cane genome were identified by the inventors.

A complete list of the identified GREP genes and proteins with their SEQ ID number and length in nucleotides (nt) or amino acids (AA) respectively, is summarized in Table 4.

TABLE 4
Plant GREP/PSK homologues: nomenclature and overview of SEQ ID NOs

| | | | Yang (Plant | | | | | |
|---|---|---|---|---|---|---|---|---|
| | | US Provisional | Phys. 2001, 127: | future name in | | | | |
| SEQ ID | | application | 842-851) | publication | | | | Accession |
| NO | | 60/283,313 | OsPSK | M. Sauter^{(a)} | Accession No.^{(b)} | EST(s)^{(c)} | BAC Gene^{(d)} | No.^{(e)} |
| 1 | cDNA | OsGREP1 | | OsPSK2 | AJ276692^{(1,2)} | C73667; D41594 | — | — |
| 2 | Protein | OsGREP1 | | | | | | |
| 3 | cDNA | OsGREP2 | | OsPSK3 | AF068333^{(1,2,3,4)} | D48790 | nbxb0068B01fg | AQ840680g |
| 4 | Protein | OsGREP2 | | | | | | |
| 5 | cDNA | OsGREP3 | | OsPSK4 | AJ276693⁽³⁾ | AU068854; | — | — |
| | | | | | | AU068855 | | |
| 6 | Protein | OsGREP3 | | | | | | |
| 7 | gDNA | OsGREP4 | | OsPSK5 | — | — | OSJNBb0009F04.16 | AAG46077.1 |
| 8 | cDNA | OsGREP4 | | | | | | |
| 9 | Protein | OsGREP4 | | | | | | |
| 10 | gDNA | AtGREP1 | | AtPSK6 | AF4228104 | — | T32N15 | 68381-69111 |
| 11 | cDNA | AtGREP1 | | | | | | |
| 12 | Protein | AtGREP1 | | | | | | |
| 13 | gDNA | AtGREP2 | AtPSK2 | AtPSK2 | AB029344 | T43923, AI996446 | T20K9.7 | AAC32433.1 |
| 14 | cDNA | AtGREP2 | | | | | | |
| 15 | Protein | AtGREP2 | | | | | | |
| 16 | cDNA | AtGREP3 | AtPSK3 | AtPSK3 | AB052752 | AA395590; | T16K5.130 | CAB66916.1 |
| | | | | | | AA720042 | | |
| 17 | Protein | AtGREP3 | | | | | | |
| 18 | gDNA | AtGREP4 | AtPSK4 | AtPSK4 | AF4228134 | — | F19F18.210 | CAB38311.1 |
| 19 | cDNA | AtGREP4 | | | | | | |
| 20 | Protein | AtGREP4 | | | | | | |
| 21 | gDNA | AtGREP5 | AtPSK1 | AtPSK1 | 1B074572 | — | F21F23.2 | AAF81285 |
| 22 | cDNA | AtGREP5 | | | | | | |
| 23 | Protein | AtGREP5 | | | | | | |
| 24 | gDNA | AtGREP6 | | AtPSK3 | | | | |
| 25 | cDNA | AtGREP6 | | | | | | |
| 26 | Protein | AtGREP6 | | | | | | |
| 27 | gDNA | AtGREP7 | | AtPSK5 | AB074574 | AF3250205 | K14B20.4 | AAG40372.1 |
| 28 | cDNA | AtGREP7 | | | | | | |
| 29 | Protein | AtGREP7 | | | | | | |
| 30 | cDNA | GmGREP1 | | GmPSK1 | BK000115⁽³⁾ | AI856752; | — | — |
| | | | | | | AI856400 | | |
| 31 | Protein | GmGREP1 | | | | | | |
| 32 | cDNA | GmGREP2 | | GmPSK2 | BK000116⁽³⁾ | BE658719; | — | — |
| | | | | | | AW156488 | | |
| 33 | Protein | GmGREP2 | | | | | | |
| 34 | cDNA | GmGREP3 | | GmPSK3 | BK000117⁽³⁾ | AW705620 | — | — |
| 35 | Protein | GmGREP3 | | | | | | |
| 36 | cDNA | GmGREP4 | | GmPSK4 | BK000118⁽³⁾ | BE820901 | — | — |
| 37 | Protein | GmGREP4 | | | | | | |
| 38 | cDNA | GmGREP5 | | GmPSK5 | BK000119⁽³⁾ | BE659360 | — | — |
| 39 | Protein | GmGREP5 | | | | | | |
| 40 | cDNA | GmGREP6 | | GmPSK6 | | | | |
| 41 | Protein | GmGREP6 | | | | | | |
| 42 | cDNA | LeGREP1 | | LePSK1 | BK000120⁽³⁾ | AI485184 | — | — |
| 43 | Protein | LeGREP1 | | | | | | |
| 44 | cDNA | LeGREP2 | | LePSK2 | BK000121⁽³⁾ | AW442998 | — | — |
| 45 | Protein | LeGREP2 | | | | | | |
| 46 | cDNA | BnGREP1 | | BnPSK1 | BK000113⁽³⁾ | H744648 | — | — |
| 47 | Protein | BnGREP1 | | | | | | |
| 48 | cDNA | ZmGREP1 | | ZmPSK1 | BK000133⁽³⁾ | AI712273 | | |
| 49 | Protein | ZmGREP1 | | | | | | |
| 50 | cDNA | ZmGREP2 | | ZmPSK2 | BK000134⁽³⁾ | BE638940; | | |
| | | | | | | BE224780 | | |
| 51 | Protein | ZmGREP2 | | | | | | |
| 52 | protein | GREP Motif | | | | | | |
| 53 | DNA | GREP Motif | | | | | | |
| 54 | cDNA | OsGREP1 | | | | | | |
| 55 | Protein | OsGREP1 | | | | | | |
| 56 | cDNA | OsGREP2 | | | | | | |
| 57 | Protein | OsGREP2 | | | | | | |
| 58 | cDNA | AtGREP4 | | | AB074572 | — | F21F23.2 | AAF81285.1 |
| 59 | protein | AtGREP4 | | | | | | |
| 60 | cDNA | AtGREP5 | AtPSK1 | AtPSK1 | 1B074572 | — | F21F23.2 | AAF81285 |
| 61 | protein | AtGREP5 | AtPSK1 | AtPSK1 | | | | |
| 62 | cDNA | GmGREP2 | | GmPSK2 | BK000116 | BE658719; | — | — |
| | | | | | | AW156488 | | |
| 63 | protein | GmGREP2 | | GmPSK2 | | | | |
| 64 | cDNA | GmGREP3 | | GmPSK3 | BK000117 | AW705620 | — | — |
| 65 | protein | GmGREP3 | | GmPSK3 | | | | |
| 66 | cDNA | ZmGREP2 | | ZmPSK2 | BK000134⁽³⁾ | BE638940; | | |
| | | | | | | BE224780 | | |
| 67 | protein | ZmGREP2 | | ZmPSK2 | | | | |
| 68 | gDNA | OsGREP5 | | | | | | |
| 69 | cDNA | OsGREP5 | | | | | | |
| 70 | Protein | OsGREP5 | | | | | | |
| 71 | gDNA | OsGREP6 | | | | | | |
| 72 | cDNA | OsGREP6 | | | | | | |
| 73 | Protein | OsGREP6 | | | | | | |
| 74 | cDNA | | | ZmPSK3 | BK000135 | BG874095; | | |
| | | | | | | T12714 | | |
| 75 | protein | | | ZmPSK3 | | | | |
| 76 | cDNA | | | ZmPSK4 | BK000136⁽³⁾ | BE344656 | — | — |
| 77 | protein | | | ZmPSK4 | | | | |
| 78 | cDNA | | | TaPSK1 | BK000131⁽³⁾ | BF474662 | — | — |
| 79 | protein | | | TaPSK1 | — | | | |
| 80 | cDNA | | | TaPSK2 | BK000132⁽³⁾ | BE423973 | — | — |
| 81 | protein | | | TaPSK2 | | | | |
| 82 | cDNA | | | PtPSK1 | BK000125⁽³⁾ | AW981986 | — | — |
| 83 | protein | | | PtPSK1 | | | | |
| 84 | cDNA | | | LePSK3 | BK000122⁽³⁾ | BE449984 | — | — |
| 85 | protein | | | LePSK3 | | | | |
| 86 | cDNA | | | LePSK4 | BK000123⁽³⁾ | BG131016 | — | — |
| 87 | protein | | | LePSK4 | | | | |
| 88 | cDNA | | | StPSK1 | BK000128⁽³⁾ | BI432554; | — | — |
| | | | | | | BI432402 | | |
| 89 | protein | | | StPSK1 | | | | |
| 90 | cDNA | | | StPSK2 | BK000129⁽³⁾ | BI433974 | — | — |
| 91 | protein | | | StPSK2 | | | | |
| 92 | cDNA | | | StPSK3 | BK000130⁽³⁾ | BI176370 | — | — |
| 93 | protein | | | StPSK3 | | | | |
| 94 | cDNA | | | SpPSK1 | BK000127⁽³⁾ | BF587258 | — | — |
| 95 | protein | | | SpPSK1 | | | | |
| 96 | cDNA | | | SbPSK1 | BK000126⁽³⁾ | BE360146 | — | — |
| 97 | protein | | | SbPSK1 | | | | |
| 98 | cDNA | | | McPSK1 | BK000124⁽³⁾ | BE130635 | — | — |
| 99 | protein | | | McPSK1 | | | | |
| 100 | cDNA | | | GaPSK1 | BK000114⁽³⁾ | BE052169 | — | — |
| 101 | protein | | | GaPSK1 | | | | |
| 102 | cDNA | | | AoPSK1 | AB033828 | | — | — |
| 103 | protein | | | AoPSK1 | | | | |
| 104 | cDNA | OsPSK | | | AB020505 | D42693; D43399 | | |
| 105 | protein | OsPSK | | | | | | |
| 106 | primer | prm0171 | | | | | | |
| 107 | primer | prm0172 | | | | | | |

gDNA: genomic DNA.
^{(a)}proposed name for plant PSK/GREP homologues.
^{(b)}accession number of deposited PSK cDNA sequence obtained either by ⁽¹⁾PCR cDNA amplification, ⁽²⁾plaque screening, ⁽³⁾EST sequence and/or EST contig assembly or ⁽⁴⁾genomic exon prediction; ⁽⁵⁾RIKEN GSC sequence, mRNA complete cds.
^{(c)}EST(s) used as source for deposited sequences. If more than two corresponding ESTs exist, only the accession numbers of ESTs used for cds prediction are shown.
^{(d)}Gene name used in annotation of bacterial artificial chromosome (BAC) sequences. When a gene is not annotated, the BAC name is given.
^{(e)}Protein accession number for the translation product of the corresponding BAC gene.
Ao, Asparagus officinalls;
At, Arabidopsis thaliana;
Bn, Brassica napus;
Ga, Gossypium arboreum;
Gm, Glycine max;
Le, Lycopersicon esculentum;
Mc, Mesembryanthemum cristallinum;
Os, Oryza sativa;
Pt, Pinus taeda;
Sb, Sorghum bicolor;
Sp, Sorghum propinquum;
St, Solanum tuberosum;
Ta, Triticum aestivum;
Zm, Zea mays.

### Example 6

### Comparative Analysis of the Peptide Sequence and Secondary Structure of GREPs

As summarized in Example 5, GREPs are typically small proteins consisting of 67 to 119 amino acids. An alignment of the full-length GREP peptide sequences is illustrated in FIG. 3 and a statistics report based on this alignment is summarized in FIG. 4. From literature sources, it was found that OsGREP1 and the other GREP proteins had some characteristics in common with the protein OsPSK described in the prior art (Yang et al., 1999). OsPSK was therefore included in this comparative analysis. The protein sequence alignment and the statistics report indicate a significant, but sometimes low, degree of conservation between all GREPs. In rice, the percentage amino acid sequence identity between the different OsGREP homologues ranges from 17 to 59%. Similarly, in A. thaliana the peptide sequence identity between the 7 GREP homologues varies from 15 to 72%. By contrast, the peptide sequence identity between OsPSK and the GREP homologues is lower and varies from 9 to 14% and from 11 to 18% in rice and A. thaliana, respectively. In general, GREPs are more distantly related to OsPSK than they are to any other member of the GREP gene families, even from different plant species. This is reflected in the phylogenetic tree calculated from the aligned sequences where the GREP proteins and OsPSK are in separate clusters (FIG. 5). The peptide sequence identity between GREPs and OsPSK is mainly restricted to a highly conserved YIYT motif at the C-terminus. Importantly, this motif is part of the pentapeptide backbone of the plant growth factor phytosulfokine-α encoded by OsPSK A second region of highly conserved sequences is found in the GREP proteins that is absent in OsPSK. This region is located 5′ of and contiguous with the YIYT motif. Together, these conserved sequences constitute a novel motif that is unique to the GREP proteins and which was termed the GREP signature motif. The GREP signature motif has the sequence CX₄₋₈^{D}/_{E}X₁₋₂CX₂₋₃^{R}/_{K}^{R}/_{K}X₄₋₅HXDYIYT^{Q}/_{H}

Many structural features are conserved between GREP proteins, confirming their relationship.

The OsGREP1 protein has a putative signal sequence for targeting to the secretory pathway, as predicted by the SignalP V1.1 software. A similar hydrophobic N-terminal region that corresponds to a putative signal sequence is predicted for all GREP proteins for which a full-length sequence is available. This finding is in agreement with the presence of a hydrophobic putative signal peptide previously documented for OsPSK (Yang et al., 2000). In addition, the secondary structure of GREP proteins is also conserved. Similar to OsGREP1, all full-length GREPs have a high probability for an α-helix that overlaps with the putative signal sequence and for three α-helices in the sequence following this region. Also, a lower probability for a turn between the first and second helix of the postleader sequence seems conserved among all GREPs (see FIG. 6). In addition, all GREPs have a central acidic domain and a short basic region at their C-terminus.

Database searches and hybridization experiments using the OsPSK gene did not lead to the identification of the subject GREP nucleic acid sequences. It was previously reported that the OsPSK protein does not have significant homology to proteins in public databases (Yang et al., 1999). This was confirmed by our BLAST searches: when the complete peptide sequence of OsPSK is used as query in blastn searches of plant databases, the OsPSK gene is identified but GREPs are not. Conversely, when using the complete peptide sequence of OsGREP1, 2 or 3 as query, other GREPs are identified while OsPSK is not. This is illustrated in Table 5, which lists the accession numbers of sequences that were retrieved in these Blast searches. In this table, AB020505 corresponds to OsPSK and AF068333 corresponds to OsGREP2. See the NCBI website for others (http://www.ncbi.nim.nih.gov/).

TABLE 5
Results of tblastn searches using the complete
OsGREP1, 2 and 3 and OsPSK coding sequence
as queries against the plant sequence database

| OsGREP1 | OsGREP2 | OsGREP3 | OsPSK |
|---|---|---|---|
| AF068333 | AF068333 | AF068333 | AB020505 |
| AC002534 | AC002534 | ATF6H11 | |
| AC004786 | ATT28I19 | AB018108 | |
| ATT28I19 | ATF19F18 | ATT16K5 | |
| ATF19F18 | ATCHRIV88 | ATT28I19 | |
| ATCHRIV88 | AC004786 | ATF19F18 | |
| ATF6H11 | AB018108 | ATCHRIV88 | |
| AB018108 | ATF6H11 | AC004786 | |
| BE820901 | ATT16K5 | AC002534 | |
| BE659360 | BE820901 | BE658719 | |
| BE802923 | BE659360 | BE802923 | |
| BE658719 | BE802923 | BE659360 | |
| AC079830 | BE658719 | BE820901 | |
| | AC079830 | AC079830 | |

Only more targeted database searches, for example, using partial OsPSK or GREP sequences, lead to the identification of both OsPSK and GREP sequences. However, these searches also result in retrieval of sequences that are unrelated to GREPs. Therefore, protein and nucleic acid sequences retrieved by Blasts were further screened for the primary and secondary structure characteristics of GREPs as disclosed herein. This approach allowed the unambiguous identification of bona fide OsGREP1 homologues as described in Example 5.

### Example 7

### Tissue Specific and Inducible Expression of OsGREP1

To determine gene expression under different conditions and in different rice tissues, mRNA abundance of OsGREP1 was analyzed by RNA gel blot hybridization. Total RNA was isolated using the TRIzol reagent (Gibco BRL) and precipitated with 4M LiCl as described (Puissant & Houdebine, 1990). The RNA was separated and blotted as described (Lorbiecke & Sauter, 1998). Hybridizations were carried out as described (Sauter, 1997). For OsGREP1, a fragment encompassing the 3′ untranslated region and a short portion of the C-terminal coding region was used as probe. This DNA fragment was random prime labelled using ³²P-dCTP. Hybridization was carried out under stringent conditions.

Gene expression was analyzed in adventitious roots 0, 2 and 6 h after submergence and in the intercalary meristem (IM), the zone of cell elongation (EZ) and the zone of cell differentiation (DZ) of the internode 0, 2, 6 and 18 h after submergence (FIG. 2). OsGREP1 gene expression levels were higher in adventitious roots than in the internode in unsubmerged rice plants. OsGREP1 expression was transiently induced in all tissues analyzed. Strongest induction was observed in adventitious roots 2 h after submergence and also in the IM and the EZ of the internode 18 and 6 h respectively after submergence. OsGREP1 expression was only slightly induced in the DZ 6 h after submergence.

mRNA abundance of OsGREP1 was also analyzed in different tissues of adult rice plants and seedlings and in suspension-cultured rice cells by RNA gel blot analysis (FIG. 7). For seedlings, the expression level of the OsGREP1 gene is generally higher in root tissue than in leaf tissue with intermediate levels in the coleoptile. OsGREP1 expression is highest in the basal part of the primary root of etiolated seedlings, which suggests that OsGREP1 gene expression is not restricted to or predominant in meristematic tissues. OsGREP1 mRNA can also be detected in suspension-cultured cells but at lower levels than in all other tissues. Growth of the internode is mediated by ethylene and ultimately regulated by gibberellin as described supra. Therefore, growth of the deepwater rice internodes can also be induced by treatment with gibberellic acid (GA₃). Stem sections containing the IM and EZ of the internode were treated with 50 μM GA₃ and analyzed for expression of OsGREP1 by RNA gel blot hybridization 0, 1, 0.5, 1, 3, 6 and 15 h after treatment (FIG. 8). Treatment of stem sections with 50 μM GA₃ resulted in a slight and transient increase in OsGREP1 transcript levels in the meristematic zone within 30 minutes and again 15 h after onset of the GA₃ treatment.

Gene expression of OsGREP1 was also determined by measuring mRNA abundance in IM sections of deepwater rice internodes treated with cycloheximide (CHX) at 0, 0.02, 2, and 20 μg/ml (FIG. 9). As shown, OsGREP1 transcripts are induced in the presence of 20 μg/ml (corresponds to 70 μM) CHX. In maize, 20 μM CHX results in 23% inhibition of protein synthesis (Berberich & Kusano, 1997) and in alfalfa 150 μM results in 90% inhibition of protein synthesis (Monroy et al., 1993). Based on this data, we infer that 70 μM CHX will inhibit protein synthesis in these experiments. These results indicate that short-lived repressors are involved in regulating OsGREP1 transcription. Alternatively, OsGREP1 mRNA is subject to degradation by a short-lived nuclease.

The foregoing results indicate that OsGREP1 mRNA levels are induced by growth promoting treatments such as submergence and GA₃ treatment, consistent with a function for the gene product as a plant growth regulator. Since expression of OsGREP1 is not restricted to meristematic tissues, i.e. the sites of active growth, it is likely that the OsGREP1 gene product is transported from its site of synthesis to its target tissue where it triggers a growth response.

### Example 8

### Using GREPs in a Two-Hybrid System to Identify Proteins involved in Growth and Development Pathways in rice and Arabidopsis

Peptides are commonly used signal molecules in animal systems and can function as triggers for signal transduction pathways by binding to specific receptors. When derived from a larger precursor, peptide hormones undergo extensive processing and modification to yield a bioactive product. Therefore, the subject GREP polypeptides can be used to identify proteins involved in maturation of the GREPs and/or to identify proteins that play a role in signalling cascades involved in plant growth and development. This can be done by using a GREP protein or a part thereof as bait, i.e. the target fused to DNA-binding domain, in a yeast two hybrid screen. A two-hybrid library has been constructed for Arabidopsis and rice. Preferentially, a rice GREP protein is used as bait to screen a rice cDNA prey library. Methods for cloning of the two-hybrid DNA-binding (bait) and activation domain (prey cDNA library) hybrid gene cassettes, yeast culture, and transformation of the yeast are all done according to well-established methods (Ausubel et al., 1990; Hannon and Bartel, 1995). Using this method, growth regulatory proteins are identified as components of the activation domain hybrid and are confirmed through sequence analysis, yeast retransformation and in vitro and in vivo plant studies.

### Example 9

### (Over)Expression of GREP Polypeptides in Transgenic Plants

In this example, the AtGREP and OsGREP genes of the present invention are expressed in transgenic rice and Arabidopsis plants. For this purpose, the constitutive promoters UbB1 and GOS2 and the seed specific promoters arcelin (Goossens et al., 1999) and prolamin (see Table I) are used for Arabidopsis and rice respectively. Other tissue-specific or tissue-preferred promoters can be used to target expression in other tissues. The GREP genes of this invention are cloned into a T-DNA cassette that has a selectable marker gene in between the T-DNA borders for selection of transformants. Agrobacterium-mediated delivery is used to introduce the T-DNA into transformation competent Arabidopsis and rice cells.

For rice, embryogenic callus derived from immature embryos is used as the target for delivery of the T-DNA. Mature dry seeds of the rice japonica cultivars Nipponbare or Taipei 309 are dehusked, sterilised and germinated on a medium containing 2,4-D (2,4-dichlorophenoxyacetic acid). After incubation in the dark for four weeks, embryogenic, scutellum-derived calli are excised and propagated on the same medium. Selected embryogenic callus is then co-cultivated with Agrobacterium. Widely used Agrobacterium strains such as LBA4404 or C58 harbouring binary T-DNA vectors may be used. The hpt gene in combination with hygromycin is suitable as a selectable marker system but other systems can be used. Co-cultivated callus is grown on 2,4-D-containing medium for 4 to 5 weeks in the dark in the presence of a suitable concentration of the selective agent. During this period, rapidly growing resistant callus islands develop. After transfer of this material to a medium with a reduced concentration of 2,4-D and incubation in the light, the embryogenic potential is released and shoots develop in the next four to five weeks. Shoots are excised from the callus and incubated for one week on an auxin-containing medium from which they can be transferred to the soil. Hardened shoots are grown under high humidity and short days in a phytotron. Seeds can be harvested three to five months after transplanting. Transformation of Arabidopsis is done by the in planta vacuum infiltration procedure (Bechthold et al., 1993).

Transgenic rice or Arabidopsis plants are allowed to flower and set seed. Morphological characteristics such as plant height, plant biomass, flowering time, the number and size of seeds are compared for transgenic plants and non-transgenic segregant siblings.

### Example 10

### Downregulation of GREP Gene Expression in Transgenic Plants

Plant genes can be specifically downregulated by antisense and co-suppression technologies. Strategies for inducing silencing of endogenous genes in plants and other organisms are well known in the art. Most procedures rely on the simultaneous expression of the sense and antisense strand of a given transcript so that the homologous endogenous gene(s) is (are) downregulated at high frequency.

Expression of one or more AtGREP and OsGREP gene(s) is downregulated in A. thaliana and O. sativa respectively, after transformation with for example a T-DNA that contains an inverted repeat of GREP gene sequences. The constructs for downregulation of target genes are made similarly as those for (over)expression, i.e. they are linked to promoter sequences and transcription termination signals. The promoters used for this purpose are constitutive promoters as well as tissue-specific or tissue-preferred promoters.

### Example 11

### The Bioactive Product Derived from GREPs Modulates Plant Growth and Development

The bioactive GREP growth regulator in a paste or liquid preparation is used to contact plant material to regulate its growth responses. Contacting the plant material is achieved by adding the formulation to growth media in in vitro cultures or ex vitrum by applying the formulation directly to plants or plant parts. Phenotypes of the contacted plant material are evaluated with respect to both growth-promoting and growth-inhibiting effects.

### Example 12

### Transgenic Plant Overexpressing OsPSK (SEQ ID NO 104)

Production of the Vector Construct

The nucleotide sequence OsPSK was amplified by RT-PCR (reverse transcriptase polymerase chain reaction) from mRNA of flowers of Arabidopsis thaliana using the “One Step Superscript” kit from Gibco (now Invitrogen). The primers we used had the following sequence: sense primer: 5′-GTGMTCCAGGMGMCAGCTAGG-3′ (prm0171, SEQ ID NO 106) and antisense primer: 5′-TTATGGGTTTTTGACATCTTGGGT-3′ (SEQ ID NO 107). The conditions for the RT-PCR were as following: 1 cycle of 30 minutes incubation at 50° C. and 2 minutes denaturation at 94° C., 35 cycles of 1 minute denaturation at 94° C., 1 minute annealing at 54 to 58° C. and 2 minutes amplification at 72° C., and 1 cycle of 5 minutes at 72° C.

The expected size of the fragment was 269 bp. PCR on the RT-PCR mix, using Pfx polymerase (Life Technologies., now Invitrogen) and the same primers as mentioned above, was used to re-amplify the fragment, under following conditions: 1 cycle of denaturation for 5 minutes at 94° C., 30 cycles of 1 minute denaturation at 94° C., 1 minute annealing at 56° C. and 1 minute amplification at 68° C., and 1 cycle of 10 minutes at 68° C.

A prominent fragment of about the expected size was isolated from gel and purified using a kit from Zymo Research. The purified fragment was subsequently kinated using a standard method. The purified and kinated PCR fragment was cloned, using standard methods, as a blunt ended fragment in the plasmid pENTR11 that was digested with NcoI and EcORV, and subsequently filled in with Pfu polymerase purchased from Promega. pENTR11 is a vector making part of the Gateway™ cloning technology, and was obtained from Life Technologies (now invitrogen) and stored in the CropDesign collection and database as p0385 (FIG. 10). The identity and base pair composition of the insert was confirmed by sequencing analysis. The resulting plasmid was quality tested using restriction digests and stored in the CropDesign plasmid collection as p0403 (FIG. 11). The p0403 vector is, according to the Gateway™ terminology, an “entry clone”, and was used as such in a standard Gateway™ LR reaction, with p0712 as “destination vector” (FIG. 12). Said p0712 vector is an in house developped vector intended for the transformation of Arabidopsis thaliana This vector contains as functional elements within the T-DNA region a selectable marker gene (herbicide resistance), a visually screenable marker gene (fluorescent marker) and a “Gateway cassette” intended for LR cloning of sequences of interest. Expression of these sequences of interest, upon the sequence being recombined into p0712, is driven by the Sunflower Ubiquitin promoter. The vector resulting from the Gateway™ LR reaction using p0403 and p0712 is p2743 (FIG. 13). This vector was controlled by control digest analysis.

Alternatively, the vector used for the phenotypic characterization of the transgenic plant containing the OsPSK gene, was constructed in another Arabidopsis expression plasmid p0427 (FIG. 20) (instead of p0712), which does not contain a visual selection marker. For cloning of this construct the procedure of amplifying the OsPSK fragement is identical as described above. The purified and kinated PCR fragment was cloned, using standard methods, as a blunt ended fragment in the plasmid pENTR11, that was digested with NcoI and EcORV, and subsequently filled in with Pfu polymerase (Promega). The identity and basepair composition of the insert was confirmed by sequencing. The resulting plasmid was quality tested using restriction digests and stored in the CropDesign plasmid collection as p0403 (FIG. 11). p0403 is, according to the Gateway™ terminology, an “entry clone”, and was used as such in a standard Gateway™ LR reaction, with p0427 as “destination vector” (FIG. 20). p0427 is an in house redeveloped vector intended for the transformation of Arabidopsis thallana. This vector contains as functional elements within the T-DNA region a herbicide resistance gene and a “Gateway cassette” intended for LR cloning of sequences of interest. Expression of these sequences of interest, upon the sequence being recombined into p0427, is driven by the Sunflower Ubiquitin promoter. The vector resulting from the Gateway™ LR reaction using p0403 and p0427 is p0531 (FIG. 22). This vector was controlled by restriction digest analysis. This vector was further used for the phenotypic characterization experiments as described below.

Transformation of the Plant Lines

Sowing and Growing of the Parental Plants

For the parental plants approximately 12 mg of wild type Arabidopsis thaliana (ecotype Columbia) seeds were suspended in 27.5 ml of 0.2% agar solution. The seeds were incubated for 2 to 3 days at a temperature of 4° C. and sown. The plants are germinated under the following standard conditions: 22° C. at day time, 18° C. at night, 65-70% RH, 20 hours of photoperiod, subirrigation with water for 15 min every 2 or 3 days. The seedlings that have developed in were than transplanted to said pots with a diameter of 5,5 cm that were prepared with a mixture of sand and peat in a ratio of 1 to 3. The plants were further grown under the same standard conditions as mentioned above.

Agrobacterium Growth Conditions and Preparation

An Agrobacterium strain C58C1 RIF with helper plasmid pMP90 containing the said p2743 vector, is inoculated in a 50 ml plastic tube containing 1 ml LB (Luria Broth) without any antibiotic. The culture is shaken for 8-9 h at 28° C. Subsequently 10 ml of LB without antibiotic is added to the said plastic tube and shaken overnight at 28° C. Afterwards the OD600 is checked. If the value is approximately 2.0, 40 ml of a 10% sucrose and 0.05% Silwet L-77 (a chemical mixture of polyalkyleneoxide modified heptamethyltrisiloxane (84%) and allyloxypolyethyleneglycol methyl ether (16%), OSi Specialties Inc) is added to the culture. The Agrobacterium culture is to be used immediately to transform the said grown plants.

Flower Dip

When each parental flower has one inflorescence of 7-10 cm of height, the inflorescences are inverted into the Agrobacterium culture and agitated gently for 2-3 seconds. 2 plants per transformation were used. Subsequently the plants were returned to the normal growing conditions as described above.

Seed Collection

5 weeks after the flower are dipped into the Agrobacterium culture, watering the plants was stopped. The plants were incubated at 25° C. and a photoperiod of 20 hours. One week later the the seeds are harvested and placed in the seed drier for one week. Subsequently the seeds are cleaned and collected in 15 ml plastic tubes. The seeds are now stored at 4° C. until further processing.

Evaluation of the Transgenic Plants Transformed with OsPSK

Selection of the Transgenic Plants

Of 11 different transgenic plant lines of Arabidopsis thaliana (named AE0017, AE0018, AE0019, AE0021, AE0022, AE0023, AE0024, AE0025, AE0026, AE0027 and Os-PSK) 500 mg of seeds is placed in 50 ml plastic tubes. 27 ml of a 0.2% agar solution is added and mixed to suspend the seeds. The said tubes are stored at 4° C. for 3 days to release dormancy. Subsequently, the suspension of seeds was evenly dispensed as drops of 50 μl on a 50 cm by 30 cm tray containing a mixture of sand and soil in a ratio of 1 to 2.

The trays were placed in the greenhouse under the following conditions: 22° C. at day time, 18° C. at night, 60% RH, 20 hours of photoperiod, subirrigation once a day with water during 15 min. The 4th day and the 10th day after sowing, the seedlings were sprayed with an herbicide solution. The 14th day after sowing, 30 resistant seedlings of each transgenic line were transplanted in individual pots with a diameter of 10 cm containing a mixture of sand and peat in a ratio of 1 to 3.

Cultivation of and Imaging of the Transgenic Plants

Said pots are then placed in the greenhouse under the same conditions as described for the trays. The pots are subirrigated during 15 min. once a week, or more if needed. The 14th, 18th, 21st, 28th, 32nd and 39th day after sowing, the rosettes of each plant were photographed using a digital camera and the pictures were stored for further analysis.

The 32nd, 38th, 41 st, 46th, 49th, and 53rd day after sowing, the inflorescence of each plant was photographed equally using a digital camera and the pictures were stored for further analysis. The number of pixels corresponding to plant tissues was recorded on each picture, converted to square cm and used as a measurement of plant size.

The 55th day after sowing, when the first siliques were ripening, a breathable plastic bag as placed on each plant and tightly attached at the base of the plants to collect the shedding seeds. The 90th day after sowing, when all the siliques were ripe, the seeds were collected and placed in a seed drier for 1 week, before storage in a sealed container at 4° C.

Results: Phenotypic Characteristics of the Transgenic Plants Transformed with OsPSK

Upon analysis of the Os-PSK plants and the other transgenic plant lines, the plants of said OsPSK plant line were on average the biggest plants found in the experiment (FIG. 17). The rozette size was slightly bigger (FIG. 14).

Upon analysis of the inflorescence, a significant difference, between 30% and 70%, was found between the average size of the inflorescences of OsPSK plants and the other transgenic lines (FIG. 15). This difference was maximal at the time of harvest Furthermore, the ratio between the size of the inflorescence before harvest and the maximal measured size of the rosette was calculated. This ratio is significantly higher in the OsPSK plants than in the other transgenic plant lines in the same experiment (FIG. 16).

### REFERENCES

Patent application No FR 2791347. Polypeptide precursor de phytosulphokine, gène le codant, cellule végétale contenant ce gène et procédé pour stimuler la prolifération des cellules végétales.

Altschul, S. F., Madden, T. L., Schaffer, A. A., Zhang, J., Zhang, Z., Miller, W., and Lipman, D. J. (1997). Gapped BLAST and PSI-BLAST: a new generation of protein database search programs. Nucleic Acids Res. 25, 3389-3402.

Altschul, S. F., Gish, W., Miller, W., Myers, E. W. and Lipman, D. J. (1990). Basic local alignment search tool. J. Mol. Biol. 215, 403-410.

An et al. (1985). EMBO J. 4, 277-284.

Armstrong et al. (1990). Plant Cell Reports 9, 335-339.

Ausubel et al. (1984). Current Protocols in Molecular Biology, Current Protocols. Standard materials and methods for plant molecular work are described in Plant Molecular Biology Labfase (1993) by R. D. D.

Ausubel (1989). Current Protocols in Molecular Biology, Green Publishing Associates and Wiley Interscience, N.Y.

Ausubel, F. M., Brent, R., Kingston, R. E., Moore, D. D., Seidman, J. G. Smith, J. A., Struhl, K. (1990). Current Protocols in Molecular Biology. John Wiley & Sons (New York).

Banerjee (1996). Biopolymers 39, 769-777.

Bayley (1992). Plant Mol. Biol. 18, 353-361.

Bechtold, N., Ellis, J., and Pelletier, G. (1993). In planta Agrobacterium-mediated gene transfer by infiltration of adult Arabidopsis thaliana plants. C. R. Acad. Sci. Paris, Life Sciences 316, 1194-1199.

Benkirane (1996). J. Biol. Chem. 271, 33218-33224.

Berberich, T., and Kusano, T. (1997). Cycloheximide induces a subset of low temperature-inducible genes in maize. Mol Gen Genet 254, 275-283.

Berry (1994). Biochem. Soc. Trans. 22, 1033-1036.

Bevan (1984). Nucleic. Acid Res. 12, 8711.

Biswas, G. C. G, Iglesias, V. A., Datta, S. K, and Potrykus, I. (1994). Transgenic Indica rice (Oryza sativa L) plants obtained by direct gene transfer to protoplasts. J. Biotechnology 32, 1-10.

Bohler (1995). Nature 376, 578-581.

Buchanan-Wollaston, V. and Ainsworth, C. (1997). Leaf senescence in Brassica napus: cloning of senescence related genes by subtractive hybridization. Plant Mol. Biol. 33, 821-834.

Chandle (1990). Physiologia Plantarum 78, 105-115.

Christou et al. (1988). Plant Physiol 87, 671-674.

Christou (1996). Trends in Plant Science 1, 423-431.

Clough et al. (1998). Plant J. 16, 735-743.

Crossway et al. (1986). Mol. Gen. Genet. 202, 179-185.

Deblaere (1985). Nucl. Acid Res. 13, 4777.

Dellaporta, S. L., Wood, V. P., and Hicks, J. B., (1983). A plant DNA mini-preparation: version II. Plant Mol Biol Reptr 1, 19-21.

Domer (1996). Bioorg. Med. Chem. 4, 709-715.

Fassina (1994). Immunomethods 5, 114-120.

Feng and Doolittle (1987). Journal of Molecular Evolution 25, 351-360.

Finn (1996). Nucleic Acids Research 24, 3357-3363.

Fraley (1986). Genetic transformation in higher plants. Crit. Rev. Plant. Sci. 4, 146.

Fritze and Walden (1995). Gene activation by T-DNA tagging. In Methods in Molecular biology 44.

Fromm et al. (1985). Proc. Natl. Acad. Sci. U.S.A. 82, 5824-5828.

Goossens, A, Dillen, W, De Clercq, J, Van Montagu, M, and Angenon, G. (1999). The arcelin-5 gene of Phaseolus vulgaris directs high seed-specific expression in transgenic Phaseolus acutifolius and Arabidopsis plants. Plant Physiol 120, 1095-1104.

Galfré (1981). Meth. Enzymol. 73, 3.

Gartland, K. M. A. and Davey, M. R., (1985). Totowa: Human Press, 281-294.

Gatz (1991). Mol. Gen. Genet. 227, 229-237.

Gerdes (1996). FEBS Lett. 389, 44-47.

Giacomin (1996). Plant Sci. 116, 59-72.

Gotoh (1997). Rinsho Byoi 45, 224-228.

Hannon, G., and Bartel, P., (1995). Identification of interacting proteins using the 2-hybrid system. Methods in Molecular and Cellular Biology 5, 289-297.

Harlow and Lane (1988). Antibodies, A Laboratory Manual, CSH Press, Cold Spring Harbor.

Hartman (1988). Proc. Natl. Acad. Sci. U.S.A. 85, 8047.

Haseloff et al., (1997). Proc. Natl. Acad. Sc. U.S.A. 94, 2122-2127.

Hayashi (1992). Science 258, 1350-1353.

Herrera-Estrella et at (1983a). Nature 303, 209-213.

Herrera-Estrella et al. (1983b). EMBO J. 2, 987-995.

Herrera-Estrella et al., (1985). In: Plant Genetic Engineering, Cambridge University Press, N.Y., pp 63-93.

Higgins and Sharp (1988). Gene 73, 237-244.

Higgings and Sharp (1989). CABIOS 5, 151-153.

Hoekema (1985). The Binary Plant Vector System, Offsetdrukkerij Kanters B. V., Alblasserdam Chapter V.

Hoffman (1995). Comput. Appl. Biosci. 11, 675-679.

Huse et al (1989). Science 246, 1275-1281.

Jefferson (1987). EMBO J. 6, 3901-3907.

Jensen (1997). Biochemistry 36, 5072-5077.

Kende, H., van der Knaap, E., and Cho, H. T. (1998). Deepwater rice: A model plant to study stem elongation. Plant Physiol 118, 1105-1110.

Klann (1996). Plant Physiol. 112, 1321-1330.

Kneller, D. G., Cohen, F. E., Langridge, R. (1990). Improvements in secondary structure prediction by an enhanced neural network. J. Mol. Biol. 214, 171-182.

Koch (1997). J. Pept. Res. 49, 80-88.

Köhler and Milstein (1975). Nature 256, 495.

Koncz (1986). Mol. Gen. Genet. 204, 383-396.

Koncz (1989). Proc. Natl. Acad. Sci. U.S.A. 86, 8467-8471.

Konez (1992). Plant Mol. Biol. 20, 963-976.

Koncz (1994). Specialized vectors for gene tagging and expression studies. In: Plant Molecular Biology Manual Vol 2, Gelvin and Schilperoort (Eds.), Dordrecht, The Netherlands: Kluwer Academic Publ. 1-22.

Krens et al (1982). Nature 296, 72-74.

Kyte, J. and Doolittle, R. F. (1982). A simple method for displaying the hydropathic character of a protein. J. Mol. Biol. 157, 105-132.

Uoyd (1994). Mol. Gen. Genet. 242, 653-657.

Lorbiecke, R. and Sauter, M. (1998). Induction of cell growth and cell division in the intercalary meristem of submerged deepwater rice (Oryza sativa L). Planta 204, 140-145.

Lyznik (1989). Plant Mol. Biol. 13, 151-161.

Maeser (1991). Mol. Gen. Genet. 230, 170-176.

Mait (1997). Transgenic Research 6, 143-156

Malmborg (1995). J. Immunol. Methods 183, 7-13.

Marsh (1984). Gene 32, 481-485.

Matsubayashi, Y. and Sakagami, Y. (1996). Phytosulfokine, sulfated peptides that induce the proliferation of single mesophyll cells of Asparagus officinalis L. Proc. Natl. Acad. Sci U.S.A. 93, 7623-7627.

Matsubayashi, Y., Takagi, L., and Sakagami, Y. (1997). Phytosulfokine-alpha, a sulfated pentapeptide, stimulates the proliferation of rice cells by means of specific high- and low-affinity binding sites. Proc. Natl. Acad. Sci. U.S.A. 94, 13357-13362.

Matsubayashi et al. (1996). Biochem Biophys Res Commun 225, 209-214.

Matsubayashi et al. (2000). J. Biol. Chem 275, 15520-15525.

Matsubayashi et al. (2001). Trends in Plant Science 6, 573-577.

McConlogue (1987). In: Current Communications in Molecular Biology, Cold Spring Harbor Laboratory ed.

Meinkoth and Wahl (1984). Anal. Biochem. 138: 267-284.

Monge (1995). J. Mol. Biol. 247, 995-1012.

Monroy, A. F., Sarhan, F., and Dhindsa, R. S. (1993). Cold-induced changes in freezing tolerance, protein phosphorylation, and gene expression. Plant Physiol 102, 1227-1235.

Murashige, T. and Skoog, F. (1962). A revised medium for rapid growth and bioassays with tobacco tissue cultures. Physiol. Plant. 15, 473-497.

Murray et al. (1989). Nucl. Acids Res. 17: 477-498.

Needleman and Wunsch (1970). J. Mol. Biol. 48, 443.

Ni (1995). Plant Journal 7, 661-676.

Nielsen, H, Engelbrecht, J, Brunak, S, and von Heijne, G. (1997). Identification of prokaryotic and eukaryotic signal peptides and prediction of their cleavage sites. Protein Eng 10, 1-6.

Olszewski (1996). Proteins 25, 286-299.

Onouchi (1991). Nucl. Acids Res. 19, 6373-6378.

Ostresh (1996). Methods in Enzynology 267, 220-234.

Pabo (1986). Biochemistry 25, 5987-5991.

Page, R. D. M. (1996). TREEVIEW: An application to display phylogenetic trees on personal computers. Computer Applications in the Biosciences 12, 357-358.

Paszkowski et al. (1984). EMBO J. 3, 2717-2722.

Paszkowski (1984). Homologous Recombination and Gene Silencing in Plants. Kluwer Academic Publishers.

Pearson and Lipman (1988). Proc. Natl. Acad. Sci. 85, 2444.

Pearson et al. (1994). Methods in Molecular Biology 24, 307-331.

Peng (1995). Plant Mol. Biol. 27, 91-104.

Potrykus and Spangenberg (1995). Gene Transfer To Plants. Springer Verlag, Berlin, N.Y.

Puissant, C. and Houdebine, L. M. (1990). An improvement of the single-step method of RNA isolation by acid guanidinium thiocyanate-phenol-chloroform extraction. Biotechniques 8, 148-149.

Raskin, I and Kende, H. (1984). Regulation of growth in stem sections of deep-water rice. Planta 160, 66-72.

Renouf (1995). Adv. Exp. Med. Biol. 376, 37-45.

Riss (1994). Plant Physiol. (Life Sci. Adv.) 13, 143-149.

Rose (1996). Biochemistry 35, 12933-12944.

Rost, B., and Sander, C. (1994). Combining evolutionary information and neural networks to predict protein secondary structure. Proteins 19, 55-72.

Rutenber (1996). Bioorg. Med. Chem. 4, 1545-1558.

Ryan, C. A. and Pearce, G. Polypeptide hormones. (2001). Plant Physiol 125, 65-68.

Sanford (1987) Particulate Science and Technology 5, 27-37.

Sambrook (1989), Molecular Cloning; A Laboratory Manual, 2nd Edition, Cold Spring Harbor Laboratory Press, Cold Spring Harbor, N.Y.

Sanger, F., Nicklen, S., and Coulson A. R. (1977). DNA sequencing with chain-terminating inhibitors. Proc. Natl. Acad. Sci. U.S.A. 74, 5463-5467.

Sauter, M. (1997). Differential expression of a CAK (cdc2-activating kinase)-like protein kinase, cyclins and cdc2 genes from rice during the cell cycle and in response to gibberellin. Plant J. 11, 181-190.

Sauter, M. and Kende, H. (1992). Gibberellin-induced growth and regulation of the cell division cycle in deepwater rice. Planta 188, 362-368.

Sauter, M., Seagull, R. M., and Kende, H. (1993). Internodal elongation and orientation of cellulose microfibrils and microtubules in deepwater rice. Planta 190, 354-362.

Schier (1996). Human Antibodies Hybridomas 7, 97-105.

Scikantha (1996). J. Bact. 178, 121.

Seifter et al. (1990). Meth. Enzymol. 182, 626-646.

Smith and Waterman (1981). Adv. Appl. Math. 2, 482.

Stultz, C. M., White, J. V., and Smith, T. F. (1993). Structural analysis based on state-space modeling. Protein Sci. 2, 305-314.

Tamura (1995). Biosci. Biotechnol. Biochem. 59, 2336-2338.

Thompson, J. D. Gibson T. J. Plewniak F. Jeanmougin F. and Higgins D. G. (1997). The ClustalX windows interface: flexible strategies for multiple sequence alignment aided by quality analysis tools. Nucleic Acids Res. 24, 4876-4882.

Tijssen (1993). Laboratory Techniques in Biochemistry and Molecular Biology-Hybridization with Nucleic Acid Probes, Part I, Chapter 2 ‘Overview of principles of hybridization and the strategy of nucleic acid probe assays’, Elsevier, N.Y.

van der Knaap E., Jagoueix, S., and Kende, H. (1997). Expression of an ortholog of replication protein A1 (RPA1) is induced by gibberellin in deepwater rice. Proc. Natl. Acad. Sci. U.S.A. 94, 9979-9983.

van der Knaap, E., Kim, J. H., and Kende, H. (2000). A novel gibberellin-induced gene from rice and its potential regulatory role in stem growth. Plant Physiol 122, 695-704.

van der Knaap, E., Song, W. Y., Ruan, D. L., Sauter, M., Ronald, P. C., and Kende, H. (1999). Expression of a gibberellin-induced leucine-rich repeat receptor-like protein kinase in deepwater rice and its interaction with kinase-associated protein phosphatase. Plant Physiol 120, 559-570.

Vasil (1993). Bio/Technology 11, 1553-1558.

Vaughan et al. (1996). Nature Biotech. 14, 309-314.

Veselkov (1996). Nature 379, 214.

Wan (1994). Plant Physiol. 104, 37-48.

Ward et al. (1989). Nature 341, 544-546.

Weiler (1997). Nucleic Acids Research 25, 2792-2799.

Wodak (1987). Ann. N.Y. Acad. Sci. 501, 1-13.

Wold F. (1983). Posftranslational Protein Modifications: Perspectives and Prospects, pp. 1-12 in Posttranslational Covalent Modification of Proteins, B. C. Johnson, Ed., Academic Press, New York.

Yang, H., Matsubayashi, Y., Hanai, H., and Sakagami, Y. (2000). Phytosulfokine-alpha, a peptide growth factor found in higher plants: its structure, functions, precursor and receptors. Plant Cell Physiol 41, 825-830.

Yang, H., Matsubayashi, Y., Nakamura, K., and Sakagami, Y. (1999). Oryza saliva PSK gene encodes a precursor of phytosulfokine-alpha, a sulfated peptide growth factor found in plants. Proc. Natl. Acad. Sci. U.S.A. 96, 13560-13565.

Zhang (1996). Biochem. Biophys. Res. Commun. 224, 327-331.

1. An isolated nucleic acid encoding a GREP growth regulating polypeptide comprising the amino acid sequence of the formula:
CX₁X₂X₃CX₄X₅X₆X₇HX₈DYIYTX₉ (SEQ ID NO 52)
wherein X₁ are 4 to 8 amino acids, X₂ is D or E, X₃ is one or two amino acids, X₄ are two or three amino acids, X₅ is R or K, X₆ is R or K, X₇ are 4 to 5 amino acids, X₈ is any amino acid and X₉ is Q or H,
or an isolated nucleic acid encoding a GREP growth regulating polypeptide comprising an amino acid sequence which is at least 90% identical to the sequence as represented in SEQ ID NO 52, or a functional fragment of such a GREP protein or polypeptide.

2. An isolated nucleic acid molecule consisting of a nucleotide sequence encoding an amino acid sequence as represented in SEQ ID NO 52, or a nucleic acid encoding an amino acid sequence which is at least 90% identical to the sequence as represented in SEQ ID NO 52.

3. The isolated nucleic acid molecule of claim 1 wherein the nucleotide sequence consists of the formula:
TGYN₁GAN₂TGYN₃MRNMRN₄CAYNNNGAYTAYATHTAYACNCAN (SEQ ID NO 53)
wherein M is A or C, R is A or G, Y is C or T, H is A or C or T, and N is G or A or T or C, and wherein N₁ is a stretch of 12 to 24 amino acid residues, N₂ is a stretch of 4 to 7 amino acid residues, N₃ is a stretch of 6 to 9 amino acid residues and N₄ is a stretch of 13 to 16 amino acid residues.

4. An isolated GREP growth regulating polypeptide encoded by a nucleic acid of claim 1.

5. The isolated GREP growth regulating polypeptide according to claim 4 consisting of an amino acid sequence as set forth in any one of SEQ ID NOs 2, 4, 6, 9, 12, 15, 17, 20, 23, 26, 29, 31, 33, 35, 37, 39, 41, 43, 45, 47, 49, 51, 52, 55, 57, 59, 61, 63, 65, 67, 70, 73, 75, 77, 79, 81, 83, 85, 87, 89, 91, 93, 95, 97, 99, 101 or 103.

6. The isolated GREP growth regulating polypeptide according to claim 4 comprising an amino acid sequence as set forth in any one of SEQ ID NOs 2, 4, 6, 9, 12, 15, 17, 20, 23, 26, 29, 31, 33, 35, 37, 39, 41, 43, 45, 47, 49, 51, 52, 55, 57, 59, 61, 63, 65, 67, 70, 73, 75, 77, 79, 81, 83, 85, 87, 89, 91, 93, 95, 97, 99, 101 or 103.

7. A vector comprising a nucleic acid encoding a plant GREP growth regulating polypeptide comprising an amino acid sequence as represented in SEQ ID NO 52, or comprising an amino acid sequence which is at least 90% identical to the sequence as represented in SEQ ID NO 52.

8. The vector according to claim 7 wherein said nucleic acid comprises the sequence as represented in SEQ ID NO 53.

9. The vector according to claim 7 wherein the GREP growth regulating polypeptide has a molecular weight in the range of from about 7 kD to about 13 kD.

10. The vector according to claim 7 wherein the GREP growth regulating polypeptide comprises a hydrophobic N-terminal leader sequence.

11. The vector according to a claim 7 wherein the amino acid sequence set forth in SEQ ID NO 52 is located near the carboxy-terminus of the GREP growth regulating polypeptide.

12. The vector according to claim 11 wherein the amino acid sequence set forth in SEQ ID NO 52 is preceded by an acidic region and followed by a basic region.

13. The vector according to claim 7 wherein the GREP growth regulating polypeptide comprises three alpha helix structures in the post leader sequence.

14. A vector comprising a nucleic acid encoding a GREP growth regulating polypeptide as defined in claim 4 or a vector comprising a nucleic acid encoding the rice growth regulating polypeptide OsPSK as represented in SEQ ID NO 105 wherein said growth regulating proteins regulate growth and/or development response in intact plants.

15. The vector according to claim 7, wherein said nucleic acid encoding a growth regulating polypeptide is under the control of a promoter which functions in plants.

16. The vector according to claim 15 wherein the promoter is a tissue-preferred or tissue-specific promoter.

17. The vector according to claim 15 wherein the promoter is an inducible or a constitutive promoter.

18. The vector according to claim 7 further comprising a terminator.

19. The vector according to claim 7 wherein said nucleic acid is a cDNA, a genomic sequence or a synthetic sequence.

20. The vector according to claim 7 wherein said nucleic acid encoding a growth regulating polypeptide is represented by at least one of SEQ ID NOs 1, 3, 5, 7, 8, 10, 11, 13, 14, 16, 18, 19, 22, 24, 25, 27, 28, 30, 32, 34, 36, 38, 40, 42, 44, 46, 48, 50, 53, 54, 56, 58, 60, 62, 64, 66, 68, 69, 71, 72, 74, 76, 78, 80, 82, 84, 86, 88, 90, 92, 94, 96, 98, 100, 102 or 104.

21. The vector according to claim 7 wherein said nucleic acid encoding a growth regulating polypeptide is in a sense or antisense orientation relative to the promoter sequence.

22. A transgenic plant, an essentially derived variety thereof, plant part, plant cell, or protoplast which comprises a nucleic acid encoding a GREP growth regulating polypeptide as defined in claim 1 or which comprises a nucleic acid encoding the rice growth regulating polypeptide OsPSK as represented in SEQ ID NO 105, wherein said nucleic acid is heterologous to the genome of said transgenic plant, or an essentially derived variety thereof, plant part, plant cell or plant protoplast.

23. A plant, essentially derived variety thereof, plant part, plant cell or protoplast wherein the plant, essentially derived variety thereof, plant part, plant cell, or protoplast has been transformed with a nucleic acid encoding a GREP growth regulating protein as defined in claim 1 or has been transformed with a nucleic acid encoding the rice growth regulating polypeptide OsPSK as represented in SEQ ID NO 105.

24. A plant, essentially derived variety thereof, plant part, plant cell, or protoplast which overexpresses a GREP growth regulating protein as defined in claim 4, or which overexpresses the rice growth regulating polypeptide OsPSK as represented in SEQ ID NO 105.

25. The plant according to claim 23 wherein the plant has been stably transformed.

26. The plant according to claim 23 wherein the plant has been transiently transformed.

27. A transgenic plant which comprises a vector according to claim 7.

28. The plant according to claim 22 wherein the plant has altered growth and/or yield and/or development characteristics.

29. The plant according to claim 22 wherein the plant has increased inflorescence.

30. The plant according to claim 22 wherein the plant has increased inflorescence of 30% to 70%.

31. The plant according to claim 22 wherein the ratio between the size of inflorescence before harvest and the maximal measured size of the leaf rosette is increased.

32. The plant according to claim 22 wherein said plant has larger seeds.

33. The plant according to any of claims 22 wherein said plant shows early vigour.

34. The plant according to any of claims 22 wherein said plant shows increased cell proliferation in early seed development.

35. Seed from the transgenic plant or essentially derived variety thereof of claim 22.

36. Pollen from the transgenic plant or essentially derived variety thereof of claim 22.

37. A harvestable part or propagation material from the transgenic plant or essentially derived variety thereof of claim 22.

38. The harvestable part of propagation material of claim 37 comprising a flower, a seed, a cutting or an explant.

39. A host cell which comprises a nucleotide sequence encoding a GREP growth regulating polypeptide claim 4 wherein said nucleotide sequence is heterologous to the genome of said host cell or wherein said host cell has been transfected or transformed with the nucleotide sequence encoding a GREP growth regulating polypeptide.

40. The host cell according to claim 39 wherein said host cell is a bacterial, yeast, fungal, or plant cell.

41. The host cell according to claim 39 wherein the nucleotide sequence encoding a GREP growth regulating polypeptide is in a sense orientation relative to a regulatory region directing expression of said nucleotide sequence.

42. The host cell according to claim 39 wherein the nucleotide sequence encoding a GREP growth regulating protein or encoding the rice growth regulating polypeptide OsPSK as represented in SEQ ID NO 105 is in an antisense orientation relative to a regulatory region directing expression of said nucleotide sequence or wherein said nucleotide sequence is included in a gene silencing construct driven by a regulatory region.

43. An isolated antisense molecule consisting of from about 14 to about 100 nucleotides targeted to the nucleotide sequence of SEQ ID NO 53.

44. An antibody which specifically recognizes a GREP plant growth regulating protein as defined in claim 4 or a fragment thereof.

45. The antibody according to claim 44 wherein the antibody is a monoclonal antibody.

46. The antibody according to claim 44 wherein the antibody is a polyclonal antibody.

47. The antibody according to claim 44 wherein said GREP fragment comprises an amino acid sequence as presented in SEQ ID NO 52, or wherein said GREP fragment comprises an amino acid sequence which is at least 90% identical to the sequence as represented in SEQ ID NO 52.

48. A method for altering growth and/or activity of a plant or plant cell which comprises modulating the level and/or activity of a GREP growth regulating polypeptide as defined in claim 4 or modulating the level and/or activity of the rice growth regulating polypeptide OsPSK as represented in SEQ ID NO 105 in the plant or plant cell.

49. The method according to claim 48 wherein the level and/or activity of a GREP growth regulating polypeptide or encoding the rice growth regulating polypeptide OsPSK as represented in SEQ ID NO 105 is increased.

50. The method according to claim 49 wherein the level and/or activity of the GREP growth regulating polypeptide or wherein the level and/or activity of the rice growth regulating polypeptide OsPSK as represented in SEQ ID NO 105 is modulated by increasing transcription of a nucleotide sequence encoding the growth regulating polypeptide.

51. The method according to claim 50 wherein the level and/or activity of a GREP growth regulating polypeptide or wherein the level and/or activity of the rice growth regulating polypeptide OsPSK as represented in SEQ ID NO 105 is decreased.

52. A method for altering growth and/or development of a plant storage organ or part thereof which comprises modulating the level and/or activity of a GREP growth regulating polypeptide as defined in claim 4 or modulating the level and/or activity of the rice growth regulating polypeptide OsPSK as represented in SEQ ID NO 105 in the storage organ or part thereof.

53. The method according to claim 52 wherein the storage organ or part thereof is a seed, root, tuber, or fruit.

54. A method for altering growth and/or development of a plant which comprises modulating the level and/or activity of a GREP growth regulating polypeptide as defined in claim 4 or modulating the level and/or activity of the rice growth regulating polypeptide OsPSK as represented in SEQ ID NO 105 in the meristem or in part thereof.

55. The method according to claim 52 wherein the level and/or activity of a GREP growth regulating protein or wherein the level and/or activity of the rice growth regulating polypeptide OsPSK as represented in SEQ ID NO 105 is increased.

56. The method according to claim 55 wherein the level and/or activity of a GREP growth regulating polypeptide 6 or wherein the level and/or activity of the rice growth regulating polypeptide OsPSK as represented in SEQ ID NO 105 is decreased.

57. The method according to claim 48 wherein the modulation of the level or activity of a GREP growth regulating polypeptide or wherein the level and/or activity of the rice growth regulating polypeptide OsPSK as represented in SEQ ID NO 105, is achieved by administering or exposing the plant or plant cells to a GREP or OsPSK growth regulating polypeptide, a homologue of a GREP or OsPSK growth regulating polypeptide, an analogue of a GREP or OsPSK growth regulating polypeptide, a derivative of a GREP or OsPSK growth regulating polypeptide, and/or to an immunologically active fragment thereof.

58. The method according to claim 52 wherein the modulation of the level or activity of a GREP growth regulating protein or wherein the level and/or activity of the rice growth regulating polypeptide OsPSK as represented in SEQ ID NO 105 is achieved by administering or exposing the plant storage organ or part thereof to a GREP or OsPSK, a homologue of a GREP or OsPSK growth regulating polypeptide, an analogue of a GREP or OsPSK growth regulating polypeptide, a derivative of a GREP or OsPSK growth regulating polypeptide, and/or to an immunologically active fragment thereof.

59. A method for downregulating levels of a GREP gene product as defined in claim 4 or the rice growth regulating polypeptide OsPSK as represented in SEQ ID NO 105, or downregulating GREP or OsPSK gene product activity, which comprises administration of GREP or OsPSK antibodies to cells, tissues, or organs of a plant, or exposing cells, tissues, or organs of a plant to GREP or OsPSK antibodies.

60. A method for downregulating levels of a GREP gene product as defined in claim 4 or downregulating levels of the rice growth regulating polypeptide OsPSK as represented in SEQ ID NO 105, or downregulating a GREP or OsPSK gene product activity which comprises expressing antibodies to the GREP or OsPSK gene product in a cell, tissue or organ of a plant.

61. A method for regulating growth and/or development of a plant or cell, tissue or organ of a plant which comprises contacting the cell, tissue, or organ of the plant with a plant GREP growth regulating polypeptide as defined in claim 4 or contacting the cell, tissue, or organ of the plant with the rice growth regulating polypeptide OsPSK as represented in SEQ ID NO 105.

62. The method according to claim 61 wherein the GREP growth regulating polypeptide or a functional fragment or bioactive peptide derived from a GREP growth regulating polypeptide is added to the growth media of the plant.

63. The method according to claim 61 wherein the GREP growth regulating polypeptide or a functional fragment or bioactive peptide derived from a GREP growth regulating polypeptide is applied directly to the plant or a part thereof as part of a formulation in a liquid or solid composition.

64. The method according to claim 61 wherein the GREP growth regulating polypeptide comprises an amino acid sequence as set forth in any one of SEQ ID NOs 2, 4, 6, 9, 12, 15, 17, 20, 23, 26, 29, 31, 33, 35, 37, 39, 41, 43, 45, 47, 49, 51, 52, 55, 57, 59, 61, 63, 65, 67, 70, 73, 75, 77, 79, 81, 83, 85, 87, 89, 91, 93, 95, 97, 99, 101 or 103.

65. The method according to claim 53 wherein the GREP growth regulating polypeptide consists of an amino acid sequence as set forth in any one of SEQ ID NOs 2, 4, 6, 9, 12, 15, 17, 20, 23, 26, 29, 31, 33, 35, 37, 39, 41, 43, 45, 47, 49, 51, 52, 55, 57, 59, 61, 63, 65, 67, 70, 73, 75, 77, 79, 81, 83, 85, 87, 89, 91, 93, 95, 97, 99, 101 or 103.

66. A peptide consisting of the amino acid sequence as represented in SEQ ID NO 52, or consisting of an amino acid sequence which is at least 90% identical to SEQ ID NO 52.

67. A method for identifying a nucleic acid molecule encoding a protein which interacts with a GREP growth regulating polypeptide, said method comprising:
(a) linking a protein encoded by a nucleic acid to a DNA-binding domain of a transcription factor; wherein the nucleic acid comprises the sequence set forth in at least one of SEQ ID NOs 1, 3, 5, 7, 8, 10, 11, 13, 14, 16, 18, 19, 22, 24, 25, 27, 28, 30, 32, 34, 36, 38, 40, 42, 44, 46, 48, 50, 53, 54, 56, 58, 60, 62, 64, 66, 68, 69, 71, 72, 74, 76, 78, 80, 82, 84, 86, 88, 90, 92, 94, 96, 98, 100 and/or 102,
(b) expressing the fusion protein of (a) in a yeast strain under the control of a promoter which is recognized by the transcription factor, wherein the yeast strain comprises a reporter gene under the control of a promoter,
(c) transforming the yeast strain of (b) with a plant cDNA library, and
(d) determining which protein or peptide encoded by a cDNA of the cDNA library interacts with the fusion of step (a) by detecting expression of the reporter gene.

68. A method for altering growth and/or development in a plant or plant cell comprising co-expression in said plant of a first nucleic acid encoding a GREP or OsPSK growth regulating polypeptide and a second nucleic acid encoding a receptor for said GREP or OsPSK growth regulating protein.

69. A method for altering growth and/or development in a plant or plant cell comprising expression in said plant of a nucleic acid encoding a GREP or OsPSK growth regulating protein in combination with modulating the functionality of the receptor for said GREP or OsPSK growth regulating protein.

70. A method for altering growth and/or development in a plant or plant cell comprising co-expression in said plant of a first nucleic acid encoding a GREP or an OsPSK growth regulating protein and a second nucleic acid encoding a protein that is involved in the post-translational processing or the biological functionality of said GREP or OsPSK growth regulating protein.

71. A method for altering growth and/or development in a plant or plant cell comprising co-expression in said plant of a first nucleic acid encoding a GREP or OsPSK growth regulating protein and a second nucleic acid encoding a protein that is involved in sulphation of said GREP or OsPSK growth regulating protein.

72. A method for altering growth and/or development in a plant or plant cell comprising co-expression in said plant of a first nucleic acid encoding a GREP or OsPSK growth regulating protein and a second nucleic acid encoding a tyrosine protein sulphotransferase.

73. A method for altering growth and/or development in a plant or plant cell comprising modulation of the activity of a GREP or an OsPSK growth regulating protein by modulating the activity of proteins involved in post-translational modifications or biological activity of said GREP or PSK growth regulating protein, such as sulphation proteins, such as tyrosine protein sulphotransferase.

74. A method according to claim 68 wherein the nucleotide sequence of said first nucleic acid is set forth in any of SEQ ID NOs 1, 3, 5, 7, 8, 10, 11, 13, 14, 16, 18, 19, 22, 24, 25, 27, 28, 30, 32, 34, 36, 38, 40, 42, 44, 46, 48, 50, 53, 54, 56, 58, 60, 62, 64, 66, 68, 69, 71, 72, 74, 76, 78, 80, 82, 84, 86, 88, 90, 92, 94, 96, 98, 100, 102 or 104.

75. A method for identifying an allele with desired features of a gene encoding a GREP growth regulating polypeptide which comprises isolating alleles for a GREP growth regulating polypeptide and testing the features of the allele by expression in a transgenic plant.

76. A method for identifying an allele of GREP growth regulating polypeptides and selecting an allele with desired features which comprises the use of genes encoding GREP growth regulating polypeptides, or sequences located in the genome in the neighbourhood of GREP genes, as molecular markers for different GREP alleles and selecting specific GREP alleles by marker-assisted breeding.

77. A method for identifying regulatory sequences of GREP growth regulating polypeptide-genes comprising:
a) hybridizing a nucleic acid encoding a GREP growth regulating polypeptide, against a plant genomic library,
b) isolating the genomic sequence corresponding to said GREP growth regulating polypeptide,
c) cloning the 5′ upstream genomic fragment of said GREP growth regulating polypeptide-gene in front of a marker gene,
d) introducing the resulting chimeric gene into a plant or plant cell for transient exression, and
e) inferring from the expression pattern the presence of a regulatory sequence in said chimeric construct.

78. An isolated nucleic acid molecule encoding a protein having an amino acid sequence as set forth in SEQ ID NO 2.

79. The isolated nucleic acid molecule of claim 78 comprising a nucleotide sequence as set forth in SEQ ID NO 1.

80. An isolated nucleic acid molecule encoding a protein having an amino acid sequence as set forth in SEQ ID NO 12.

81. The isolated nucleic acid molecule of claim 80 comprising a nucleotide sequence as set forth in SEQ ID NO 10 or SEQ ID NO 11.

82. An isolated nucleic acid molecule encoding a protein having an amino acid sequence as set forth in SEQ ID NO 70.

83. The isolated nucleic acid molecule of claim 82 comprising a nucleotide sequence as set forth in SEQ ID NO 69 or SEQ ID NO 68.

84. An isolated nucleic acid molecule encoding a protein having an amino acid sequence as set forth in SEQ ID NO 73.

85. The isolated nucleic acid molecule of claim 84 comprising a nucleotide sequence as set forth in SEQ ID NO 72 or SEQ ID NO 71.
